(19) United States
(12) Patent Application Publication
(10) Pub. No.: US 2026/0234703 A1
(43) Pub. Date: Aug. 13, 2026

(54) METHODS AND COMPOSITIONS FOR RECORDING BINDING INTERACTIONS

(71) Applicant: Nautilus Subsidiary, Inc., Seattle, WA (US)

(72) Inventors: Robert GROTHE, San Jose, CA (US); Fletcher BAIN, Redwood City, CA (US); Kara JUNEAU, San Carlos, CA (US); Rajesh KOTA, San Diego, CA (US); Phillip BRERETON, Belmont, CA (US); Keith BJORNSON, Fremont, CA (US); Steven TAN, San Mateo, CA (US); Terren R. CHANG, Redwood City, CA (US)

(21) Appl. No.: 19/535,751

(22) Filed: Feb. 10, 2026

Related U.S. Application Data
(60) Provisional application No. 63/757,637, filed on Feb. 12, 2025.

(51) Int. Cl. C12Q 1/6834

(52) U.S. Cl. CPC C12Q 1/6834

(57) ABSTRACT

Methods, compositions, and systems for detecting or characterizing analytes are provided. In certain configurations set forth herein, the methods can employ detectable probes comprising affinity reagents attached to secondary detection moieties. In certain configurations set forth herein, the methods can form structures that improve the emission of detectable optical signals.

### CROSS-REFERENCE

This application claims priority to U.S. Provisional Application No. 63/757,637, filed on Feb. 12, 2025, which is incorporated herein by reference in its entirety.

### BACKGROUND

Affinity reagents include a broad class of reagents that form detectable interactions with analytes and other molecules. Affinity reagents can be configured to form reversible bound complexes with analytes. The observation of binding between an affinity reagent and analyte can provide useful information for characterizing the structure and properties of the analyte based on known recognition properties of the affinity reagent. Substantial resources and effort are typically invested into producing affinity reagents that bind to analytes of interest with sufficient specificity to distinguish the analytes in complex mixtures, such as biopsy samples, which typically contain a large number and variety of proteins, nucleic acids and other biochemical analytes. The specificity and strength of binding between an affinity reagent and analyte can be heavily influenced by small changes in conditions used for the assay such as changes in temperature, ionic strength, pH, and concentration of affinity reagent and analyte. This combination of factors can constrain the design of multiplex assays in which a large number of different analytes are to be evaluated, in parallel, for binding to a given affinity reagent. The present disclosure provides compositions and methods that improve binding assays and provide advantages that extend to multiplexed formats. Other advantages are provided as well.

### SUMMARY

In an aspect, provided herein is a method, comprising: (a) delivering a fluidic medium from a first vessel to a second vessel, wherein the fluidic medium comprises a plurality of detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe comprises: (i) an affinity reagent comprising a binding region and a non-binding region, (ii) a molecule non-covalently bound to the non-binding region of the affinity reagent, and (iii) a detectable label attached to the molecule, (b) binding detectable probes of the plurality of detectable probes to analytes of the plurality of analytes, and (c) detecting at single-analyte resolution for each site of the plurality of sites a presence or absence of a signal from the detectable label of a detectable probe of the plurality of detectable probes.

In another aspect, provided herein is a method, comprising: (a) delivering a fluidic medium from a first vessel to a second vessel, wherein the fluidic medium comprises a plurality of first detectable probes and a plurality of second detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe of the plurality of first detectable probes and the plurality of second detectable probes comprises: (i) an affinity reagent comprising a binding region and a non-binding region, (ii) a molecule non-covalently bound to the non-binding region of the affinity reagent, and (iii) a detectable label attached to the molecule, (b) binding detectable probes of the plurality of first detectable probes and plurality of second detectable probes to analytes of the plurality of analytes, and (c) detecting at single-analyte resolution for each site of the plurality of sites a presence or absence of a signal from the detectable label of a detectable probe of the plurality of first detectable probes and the detectable label of a detectable probe of the plurality of second detectable probes, wherein the non-binding region of the affinity reagents of the detectable probes of the plurality of first detectable probes differ from the non-binding region of the affinity reagents of the detectable probes of the plurality of second detectable probes, and wherein the detectable labels of the detectable probes of the plurality of first detectable probes differ from the detectable labels of the detectable probes of the plurality of second detectable probes.

In another aspect, provided herein is a system, comprising: (a) a first vessel, wherein the first vessel comprises a fluidic medium, wherein the fluidic medium comprises a plurality of detectable probes, wherein each detectable probe comprises: (i) an affinity reagent comprising a binding region and a non-binding region, (ii) a molecule non-covalently bound to the non-binding region of the affinity reagent, and (iii) a detectable label attached to the molecule, (b) a second vessel comprising a solid support, wherein the solid support comprises a plurality of sites, wherein a plurality of analytes is immobilized on the plurality of sites, wherein each site of the plurality of sites contains only one analyte of the plurality of analytes, (c) a fluid transfer device, wherein the fluid transfer device provides fluidic communication between the first vessel and the second vessel, (d) an optical detector, and (e) one or more processors programmed to read data from the optical detector to detect presence or absence of a signal from the detectable label at each site of the plurality of sites at single-analyte resolution.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1 shows a cross section of a region of a flow cell having two protein analytes (white globules) immobilized on the lower surface and a fluid phase containing affinity reagents (Y shapes), wherein the affinity reagents are attached to a first luminophore (open circle) and the protein analytes are attached to a second luminophore (closed circle), in accordance with some embodiments.

FIGS. 2A, 2B, 2C, 2D, 2E, 2F, 2G, and 2H show various configurations of systems for recording binding interactions utilizing a polymerization or depolymerization agent, in accordance with some embodiments.

FIG. 3 depicts a schematic of a system that is configured to implement certain methods set forth herein, in accordance with some embodiments.

FIGS. 4A, 4B, and 4C show steps of a method of detecting a binding interaction between an affinity reagent and an analyte utilizing a secondary affinity reagent, in accordance with some embodiments.

FIGS. 5A, 5B, 5C, and 5D show steps of a method of detecting a binding interaction between an affinity reagent and an analyte utilizing a secondary affinity reagent, in accordance with some embodiments.

FIGS. 6A, 6B, 6C, 6D, 6E, 6F, and 6G show various configurations of systems for forming kinetic traps comprising affinity reagents, in accordance with some embodiments.

FIGS. 7A and 7B illustrate differences in observation of binding equilibrium between a plurality of affinity reagents and a plurality of analytes when the binding interactions are not recorded (upper) or are recorded (lower), in accordance with some embodiments.

FIGS. 8A, 8B, 8C, 8D, and 8E depict configurations of a system for Forster Resonance Energy Transfer (FRET)-based detection that associates an acceptor fluorescent molecule to a plurality of donor fluorescent molecules, in accordance with some embodiments.

FIGS. 9A, 9B, and 9C show isometric views of relative spatial configurations of one or more acceptor fluorescent molecules relative to a plurality of donor fluorescent molecules, in accordance with some embodiments.

FIGS. 10A and 10B show ELISA fluorescence measurements for multiplexed mixtures of affinity reagents labeled via secondary detection moieties.

FIG. 11 displays co-localization data for detectable probes targeting dimer and trimer epitopes against a single-molecule array of human transferrin.

### DETAILED DESCRIPTION

A fundamental challenge to the design and implementation of binding assays is to detect binding of an affinity reagent to an analyte of interest in the presence of an excess of non-bound affinity reagent and/or non-bound analyte. Typically, a binding assay includes an incubation period wherein at least one reactant (e.g., affinity reagent and/or analyte) is present at a relatively high concentration in solution. During this incubation period, an equilibrium is typically established between three species: non-bound affinity reagent, non-bound analyte and analyte: affinity reagent complex. For many assays, the complex is detected via a label that is attached to the affinity reagent or analyte. However, one or both of the non-bound species are typically present in a large excess compared to the complex, and this can result in a level of background signal from non-bound species that overwhelms detection of signal from the complexes. One approach to reducing unwanted background signal is to separate non-bound species from complexes, for example, by removal of the non-bound species from the assay, prior to detection. However, removal of the non-bound species results in a disequilibrium condition wherein, depending upon the kinetics of the binding reaction, the complexes may begin to dissociate. Substantial degrees of dissociation prior to or during the timeframe of detection create a risk of anomalous results.

Binding equilibrium represents a dynamic balance between association and dissociation of molecules. For example, in considering the binding equilibrium between a first species and a second species, observation of complexed molecules and unbound molecules will provide a steady-state measurement of the equilibrium, but over a long enough time, virtually all molecules of the first species and the second species will at least briefly form a complex with the other species. Observation of binding at equilibrium between molecules may provide incomplete information due to not observing all binding interactions that occurred.

The present disclosure provides binding assays that are used to form a complex between an affinity reagent and analyte, optionally with at least one of the affinity reagent and analyte labeled in the non-bound state, and detecting the complex via the detection of the label. Alternatively, some binding assay may introduce a third molecule that binds to the complex formed by the affinity reagent and analyte, in which the third molecule optionally contains a label. In some configurations, the binding assays allow detection of the labeled complex in the presence of non-bound species that also contain the label. In some cases, non-bound species are in substantial excess compared to the concentration or amount of complex present during a detection step. A binding assay of the present disclosure can be configured to detect complexes under an equilibrium condition, a disequilibrium condition, or in a simulated equilibrium condition.

The present disclosure provides a method of detecting a first reaction, comprising: (a) providing immobilized on a solid support: (i) an analyte, and (ii) a first reactant, the first reactant being immobilized on the support within a first distance from the analyte, (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant, (c) forming a first reaction between the analyte and the affinity reagent, and forming a second reaction between the first reactant and the second reactant, and (d) detecting the first reaction. The present disclosure further provides a method of detecting a first reaction, comprising: (a) providing immobilized on a solid support: (i) an analyte, and (ii) a first reactant, the first reactant being immobilized on the support within a first distance from the analyte, (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant, (c) forming a first reaction between the analyte and the affinity reagent, thereby bringing the second reactant within the second distance of the first reactant, (d) after forming the first reaction, forming the second reaction between the first reactant and the second reactant, and (e) detecting the first reaction.

Terms used herein will be understood to take on their ordinary meaning in the relevant art unless specified otherwise. Several terms used herein and their meanings are set forth below.

In some of the implementations described herein, the term “address,” when used in reference to an array, can mean a location in an array occupied by, or configured to be occupied by, a particular molecule or analyte such as a protein, nucleic acid, structured nucleic acid particle or reactive moiety. An address can contain a single analyte molecule, or it can contain a population of several analyte molecules of the same species (i.e. an ensemble of the molecules). Alternatively, an address can include a population of molecules that are different species such as an attached analyte and a linked affinity reagent. Addresses of an array are typically discrete. The discrete sites can be contiguous, or they can have interstitial spaces between each other. An array useful herein can have, for example, addresses that are separated by less than 100 microns, 10 microns, 1 micron, 0.5 micron, 0.1 micron, 0.01 micron or less. Alternatively or additionally, an array can have addresses that are separated by at least 0.01 micron, 0.1 micron, 0.5 micron, 1 micron, 10 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁷, 1×10⁸, 1×10⁹, 1×10¹⁰, 1×10¹¹, 1×10¹², or more addresses, some or all of which are occupied by analytes or molecules. An address that is configured to bind an analyte, for example by the presence of attachment moieties, is referred to herein as a “site.”

In some of the implementations described herein, the term “affinity reagent” can refer to a molecule or other substance that is capable of specifically or reproducibly binding to an analyte (e.g., protein) or moiety (e.g., post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include chemically reactive affinity reagents, catalytic affinity reagents (e.g., kinases, proteases, etc.) or chemically non-reactive affinity reagents (e.g., antibodies or fragments thereof). An affinity reagent can be chemically non-reactive and non-catalytic, thereby not permanently altering the chemical structure of an analyte to which it binds. Affinity reagents that can be particularly useful for binding to proteins include, but are not limited to, antibodies such as full-length antibodies or functional fragments thereof (e.g., Fab′ fragments, F(ab′)₂ fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

In some of the implementations described herein, the term “antibody” can refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some of the implementations described herein, the term “array” can refer to a population of analytes (e.g., proteins) that are co-localized with unique identifiers such that the analytes can be distinguished from each other. A unique identifier can be a solid support (e.g., particle or bead), structured nucleic acid particle (SNAP), address on a solid support, tag, label (e.g., luminophore), or barcode (e.g., nucleic acid barcode) that is co-localized with an analyte and that is distinct from other identifiers in the array. Analytes can be co-localized with unique identifiers by attachment, for example, via covalent or non-covalent bonds. An array can include different analytes that are each attached to different unique identifiers. An array can include different unique identifiers that are attached to the same or similar types of analytes. An array can include separate solid supports, or separate sites on a given solid support, that each bear a respective analyte, wherein the respective analytes can be identified according to the locations of the solid supports or sites. Analytes that can be included in an array can be, for example, nucleic acids such as structured nucleic acid particles, proteins, enzymes, glycans, affinity reagents, ligands, or receptors.

In some of the implementations described herein, the term “artificial” when used in reference to a substance, can mean that the substance is made by human activity rather than occurring naturally. For example, a polymer that is made at least in part by human activity or that includes at least one artificial monomer unit is referred to as an “artificial polymer.” As used herein, the term “natural” when used in reference to a substance, means that the substance occurs naturally. A substance that is purified or refined from its naturally occurring state without further modification can be considered natural, but a substance that is chemically modified from its naturally occurring state can be considered artificial. For example, cellulose can be considered a natural polymer, but methyl cellulose can be considered an artificial polymer.

In some of the implementations described herein, the term “attached” can refer to the state of two things being joined, fastened, adhered, connected or bound to each other.

Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some of the implementations described herein, the term “binding affinity” or “affinity” can refer to the strength or extent of binding between an affinity reagent and a binding partner. The binding affinity of an affinity reagent for a binding partner may be qualified as being “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

In some of the implementations described herein, the term “binding interaction” can refer to a reaction that associates an affinity reagent to an analyte. A binding reaction may be a covalent or non-covalent interaction. A binding interaction may associate an affinity reagent to an analyte for a sufficient length of time to detect a complex formed by the affinity reagent and analyte.

In some of the implementations described herein, the term “biomolecule” can refer to a molecule that is produced by a living organism or capable of being produced by a living system. Biomolecules include, for example, proteins, amino acids, nucleic acids, nucleotides, nucleosides, polysaccharides, glycans, sugars, hormones, metabolites and the like. Biomolecules can encompass both natural and artificial biomolecules.

In some of the implementations described herein, the term “complex” can refer to two or more molecules held together by at least one non-covalent interaction. Two or more molecules of a complex can be dissociated from each other without necessarily breaking any covalent bonds. Rather, two or more molecules of a complex can be dissociated by breaking at least one non-covalent bond. It will be understood that in some cases covalent bonds can also be broken when dissociating two or more molecules of a complex.

The term “comprising” is intended herein to be open-ended, including not only the recited elements, but further encompassing any additional elements.

In some of the implementations described herein, the term “covalent,” when used in reference to a bond between atoms or moieties of a molecule, can refer to bonding due to sharing of a pair of electrons between the two atoms or moieties. Covalent interaction can arise due to a chemical reaction between a first reactive moiety and a second reactive moiety, optionally in the presence of a third intermediary or catalytic moiety. Covalent binding interactions can form between two atoms or moieties due to various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can also form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 and 11,505,796, each of which is herein incorporated by reference in its entirety.

In some of the implementations described herein, the term “docker” can refer to a molecule or moiety that is configured to interact with a tether or that is interacting with a tether. A docker can be a moiety of a substance, object, molecule, solid support, address, particle, or bead. A docker can include a polymer, nucleic acid strand, nucleic acid duplex, nucleotide sequence, protein, affinity reagent, epitope, paratope, receptor, ligand or the like. A docker can interact with a tether via covalent or non-covalent bonding.

In some of the implementations described herein, the term “each,” when used in reference to a collection of items, can be intended to identify an individual item in the collection but does not necessarily refer to every item in the collection. Exceptions can occur if explicit disclosure or context clearly dictates otherwise.

In some of the implementations described herein, the term “epitope” can refer to a molecule or part of a molecule, which is recognized by or binds specifically to an affinity reagent or paratope. Epitopes may include amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, miniprotein or other affinity reagent. An epitope can optionally bind an antibody to elicit an immune response. However, an epitope need not necessarily participate in, nor be capable of, eliciting an immune response.

In some of the implementations described herein, the term “fluid-phase” or “fluid phase,” when used in reference to a molecule or particle participating in a binding interaction, can mean the molecule or particle is in a state wherein it is mobile in a fluid, for example, being capable of diffusing through the fluid to another molecule or particle that is complementary to the molecule or particle in the binding interaction. A fluid-phase molecule or particle may be attached to a solid support provided the moiety that attaches the molecule or particle to the solid support permits sufficient movement of the molecule or particle through the fluid to contact another molecule or particle that is complementary to the molecule or particle in a binding interaction. A fluid-phase molecule or particle may be attached to a solid support by a linker, such as a flexible linker. An immobilized analyte that is provided in a denatured or partially-denatured state may contain one or more fluid-phase portions of its molecular structure.

In some of the implementations described herein, the term “gap,” when used in reference to a double stranded nucleic acid, can refer to a region in one strand of the double helix which occurs between two double stranded regions and which lacks at least one nucleotide, whereby the gap is incapable of forming a Watson-Crick base pair with the other strand of the double helix. A gap in one strand of a double helix can span at least 1, 2, 3, 4, 5, 10, 15, 20, 25 or more nucleotide positions of the other strand of the double helix.

In some of the implementations described herein, the term “hairpin sequence” can mean a nucleotide sequence that includes a first region of the nucleotide sequence that is the reverse complement of a second region of the nucleotide sequence. The first region of the nucleotide sequence and second region of the nucleotide sequence are typically separated by an intermediate region that forms a single-stranded structure, that is referred to as a “loop,” when the nucleotide sequence self-anneals. The hairpin sequence can include a palindromic sequence of nucleotides or a gapped palindromic sequence of nucleotides. The gap in the palindrome can occur in the loop and/or portions of the palindromic sequence can occur in the loop. The reverse complementary regions of a hairpin sequence can anneal with the second region of the nucleotide sequence to form an intramolecular double helix that is referred to as a “stem” structure.

In some of the implementations described herein, the term “immobilized,” when used in reference to a molecule or particle that is in contact with a fluid phase, can refer to the molecule or particle or a portion thereof being prevented from diffusing in the fluid phase. For example, immobilization can occur due to confinement at, or attachment to, a solid phase. Immobilization can be temporary (e.g., for the duration of one or more steps of a method set forth herein) or permanent. Immobilization can be reversible or irreversible under conditions utilized for a method, system or composition set forth herein. A denatured molecule may be considered immobilized if the molecule as a whole cannot diffuse through the fluid phase, even if portions of the molecular structure have an ability to diffuse in regions of the fluid adjacent to the immobilization site of the molecule.

In some of the implementations described herein, the terms “label” and “detectable label” can refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g., fluorophore), chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., sequencing of, or hybridization to, a nucleic acid barcode) or in a time-resolved manner (e.g., time-resolved fluorescence). A label may produce a signal with a characteristic frequency, intensity, polarity, duration, wavelength, sequence, or fingerprint.

In some of the implementations described herein, the terms “linker” and “linking moiety” can refer synonymously to a moiety that connects two objects to each other. One or both objects can be a molecule (e.g., affinity reagent or analyte), solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, polypeptide, nucleotide, nucleic acid, nucleic acid origami, dendrimer, peptide nucleic acid (PNA), polysaccharide, carbon, nitrogen, oxygen, ether, sulfur, or disulfide. A linker can be a bead or particle such as a structured nucleic acid particle.

In some of the implementations described herein, the term “moiety” can refer to a component or part of a molecule. The term does not necessarily denote the relative size of the component or part compared to the rest of the molecule, unless indicated otherwise. A moiety can include one or more atoms.

In some of the implementations described herein, the term “non-covalent,” when used in reference to a bond between atoms or moieties of a molecule, can refer to bonding due a mechanism other than electron pair-sharing between the two atoms or moieties. Non-covalent interaction can arise due to an electrostatic or magnetic interaction between moieties and/or atoms. Non-covalent binding interactions can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent interaction may include hybridization of a first oligonucleotide to a complementary second oligonucleotide. In some cases, a non-covalent interaction may form between a receptor and ligand, such as streptavidin-biotin. Other useful non-covalent interactions can include affinity reagent-target interactions, such as antibody-epitope or aptamer-epitope interactions.

In some of the implementations described herein, the term “nucleic acid origami” can refer to a nucleic acid construct having an engineered tertiary or quaternary structure. A nucleic acid origami may include DNA, RNA, PNA, or modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some of the implementations described herein, the term “paratope” can refer to a molecule or part of an affinity reagent, which recognizes or binds to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

In some of the implementations described herein, the term “particle” can mean an object having a largest dimension between 10 nm and 1 mm. The object can be composed of a rigid or semi-rigid material. The particle can be insoluble in a fluid such as aqueous liquid. A particle can have a shape characterized, for example, as a sphere, ovoid, polyhedron, or other recognized shape whether having regular or irregular dimensions. Exemplary particles include, but are not limited to, structured nucleic acid particles (SNAPs) such as nucleic acid origami particles; optically detectable particles such as fluorescent nanoparticles, FluoSpheres™, and quantum dots; organic particles; inorganic particles; viral particles, such as phage particles having analytes displayed on their surfaces; gel particles; or particles made from solid support materials set forth herein or known in the art.

In some of the implementations described herein, the term “polymer” can refer to a molecule having a plurality of monomer subunits connected via a network of covalent bonds. The network may contain a single type of monomer subunit, or two or more types of monomer subunits. A polymer network may have a pattern of subunits or a random network of subunits (e.g., a protein). The network can include one or more chain(s) of the monomer subunits. A polymer can be linear or branched. A linear polymer includes only one chain in the network of covalent bonds. A branched polymer includes at least two chains in the network of covalent bonds. For example, a branched polymer can include at least 2, 3, 4, 5, 6, 8, 10 or more chains in the network of covalent bonds. Alternatively or additionally, a branched polymer can include at most 10, 8, 6, 5, 4, 3 or 2 chains in the network of covalent bonds. A polymer can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

In some of the implementations described herein, the term “pool,” when used in reference to a plurality of objects or molecules, can refer to the objects or molecules being in fluidic communication with each other. A pool can be in a fluid phase that is, in turn, in contact with a solid phase. For example, the solid phase can include immobilized objects or substances that are in communication with fluid-phase objects or substances.

In some of the implementations described herein, the term “protein” can refer to a molecule including three or more amino acids joined by peptide bonds. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

In some of the implementations described herein, the term “recognize” can refer to the capability of two or more molecules to interact with each other through non-covalent bonding such as hydrogen bonding, metal coordination, hydrophobic forces, van der Waals forces, T-T interactions, halogen bonding, or resonant interaction effects.

In some of the implementations described herein, the term “reaction,” when used in reference to a first molecule, particle, or moiety and a second molecule, particle, or moiety, can refer to a chemical interaction that involves both molecules, particle, or moieties. A reaction can include reversible or irreversible binding of a first molecule, particle, or moiety to the second molecule, particle or moiety. A reaction can include transfer of matter or energy from the first molecule, particle, or moiety to the second molecule, particle or moiety, such as transfer of one or more atoms, or transfer of a photon. A reaction can include a catalyzed change to the second molecule, particle, or moiety when it is associated to the first molecule, particle, or moiety. A reaction can include interactions of biomolecules, such as ligand-receptor binding, nucleic acid hybridization, protein-protein interactions, protein-nucleic acid interactions, etc.

In some of the implementations described herein, the term “recording,” when used in reference to a binding interaction, can refer to altering the presence of a detectable label at a unique identifier associated with an analyte, thereby providing a characteristic signal at the unique identifier until a detection event has occurred. Recording can include providing the detectable label to the unique identifier, thereby providing a characteristic presence of a signal from the detectable label at the unique identifier when the binding interaction has occurred at the unique identifier. Recording can include removing the detectable label from the unique identifier, thereby providing a characteristic absence of a signal from the detectable label at the unique identifier when the binding interaction has occurred at the unique identifier. Recording can include providing a reaction that binds an affinity reagent to an analyte in the presence of a detectable label until a detection event has occurred. Recording can include transferring a detectable label between an affinity reagent and an analyte or unique identifier, thereby providing a characteristic signal at the unique identifier until a detection event has occurred. A characteristic signal provided by the recording of a binding interaction may persist for any number of intermediate method steps that occur between the altering of a detectable label and a detection event of the characteristic signal.

In some of the implementations described herein, the term “quenched,” when used in reference to a label, can mean the label is inhibited or prevented from producing a detectable signal. For example, a luminophore can be quenched by physical proximity to a quenching agent that inhibits the luminophore from producing a detectable photon when excited by an appropriate wavelength of light. In other examples, a fluorophore can be quenched by excitation with light at a wavelength that differs from an excitatory wavelength for the fluorophore.

In some of the implementations described herein, the term “retaining component” can refer to a particle, molecule or material to which one or more moieties of an affinity reagent or analyte are attached. Exemplary retaining components include, but are not limited to, structured nucleic acid particles, nucleic acid origami, particles made of solid support materials, or polymers such as branched polymers or dendrimers. Affinity reagent moieties that can be attached to a retaining component, directly or indirectly, include for example, one or more paratopes, one or more labels, one or more antibodies, one or more nucleic acid aptamers, one or more nucleic acid tags or the like.

In some of the implementations described herein, the term “single,” when used in reference to an object such as an analyte, can mean that the object is individually manipulated or distinguished from other objects. A single object can also be referred to as one, and only one, object. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some of the implementations described herein, the term “single-analyte resolution” can refer to the detection of, or ability to detect, an analyte on an individual basis, for example, as distinguished from its nearest neighbor in an array. The term when used in reference to a single-analyte array, refers to detection of a single-analyte under the conditions that: 1) the single-analyte is detected by a signal with a magnitude that exceeds the magnitude of background signals for the detection system, and 2) the single-analyte is detected by a signal at a location that is spatially separated from the location of a signal corresponding to a different single-analyte (i.e., a spatial minimum of signal magnitude exists between a first single-analyte and a second single-analyte for the two single-analytes to be spatially resolved). In some cases, a signal corresponding to a first single-analyte may be considered spatially resolved from a signal corresponding to a second single-analyte if a signal minimum occurs between the locations of the two single-analytes with a magnitude that is substantially less than an average or peak signal maximum of one or both signal maxima corresponding to the first and second single analytes. For example, a signal minimum between two signal maxima corresponding respectively to a first single analyte and a second single analyte may have a magnitude that is no more than about 95%, 90%, 80%, 70%, 60%, 50%, 40%, 30%, 20%, 10%, 5%, 1%, or less than 1% of an average or peak signal maximum of the two signal maxima. In some cases, signals corresponding to two or more analytes may be considered spatially resolved if a spatial resolution criterion is achieved, such as the Rayleigh Criterion. A signal magnitude (peak or average) corresponding to a single-analyte may have a signal-to-noise ratio relative to an average background signal of at least about 1.1:1, 1.5:1, 2:1, 2.5:1, 3:1, 4:1, 5:1, 10:1, 20:1, 50:1, 100:1 or more than 100:1.

In some of the implementations described herein, the term “solid support” can refer to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass, modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon, quartz, fused silica and modified silicon, carbon, metals, inorganic glasses, optical fiber bundles, and polymers.

In some of the implementations described herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure (i.e. the helical twist or direction of the polynucleotide strand) of a SNAP can be configured to be denser than a nucleic acid molecule of similar length in a random coil or other non-structured state. A SNAP can optionally be modified to permit attachment of additional molecules to the SNAP. A SNAP may contain DNA, RNA, PNA, or modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “tether” refers to a molecule or moiety that is configured to interact with a docker or that is interacting with a docker. A tether can be a moiety of a substance, object, molecule (e.g., affinity reagent or analyte), solid support, address, particle, or bead. A tether can include a polymer, nucleic acid strand, nucleic acid duplex, nucleotide sequence, protein, affinity reagent, epitope, paratope, receptor, ligand or the like. A tether can interact with a docker via covalent or non-covalent bonding.

In some of the implementations described herein, the term “unique identifier” can refer to a moiety, object or substance that is co-localized with an analyte and that is distinct from other identifiers, throughout one or more steps of a process. The moiety, object or substance can be, for example, a solid support such as a particle or bead; a location on a solid support; a site in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to a co-localized analyte, for example, being synthetically attached to the co-localized analyte. Alternatively, a unique identifier can be endogenous to the analyte, for example, being attached or co-localized with the analyte in the native milieu of the analyte.

In some of the implementations described herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g., in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

Methods set forth herein may comprise a step of forming a reaction between a first reactant and a second reactant. The reaction may require bringing the first reactant into a sufficient proximity to the second reactant to form the reaction. Sufficient proximity between a first reactant and a second reactant can depend upon the nature of the interaction between the pair. A reaction may occur at a nanometer-scale separation distance between the first reactant and the second reactant (e.g., a photon transfer reaction between a dye pair), or may occur at an Angstrom-scale separation distance (e.g., covalent bond formation, certain electrostatic non-covalent interactions). A separation distance between a first reactant and a second reactant may be no more than about 50 nanometers (nm), 40 nm, 30 nm, 20 nm, 10 nm, 5 nm, 1 nm, 0.5 nm, 0.1 nm, 0.05 nm, 0.01 nm, or less than 0.01 nm. Alternatively or additionally, a separation distance between a first reactant and a second reactant may be at least about 0.01 nm, 0.05 nm, 0.1 nm, 0.5 nm, 1 nm, 5 nm, 10 nm, 20 nm, 30 nm, 40 nm, 50 nm, or more than 50 nm. In some cases, a separation distance between a first reactant and a second reactant may be less than a separation distance between an analyte and the first reactant, in which the analyte and first reactant are co-localized at a same address.

A first reactant that is configured to react with a second reactant may be co-located with an analyte at an address of a solid support. There may be a separation distance between the first reactant and the analyte at an address, for example as measured by the distance between their respective attachment points to the address. Alternatively, a reactive moiety of a first reactant may be attached by a linker to a solid support, thereby facilitating some diffusion of the reactive moiety adjacent to a co-located analyte. In such case, the separation distance between the first reactant and the analyte may be an average distance between the analyte and the reactive moiety of the first reactant. The separation distance between a first reactant and an analyte at an address may be chosen to balance between the selectivity of the reaction between the first reactant and the second reactant and the reactivity of the first reactant. For example, it may be preferable to attach a first reactant in close proximity to an analyte to decrease the likelihood that the first reactant binds to a second reactant when the affinity reagent attached to the second reactant is not bound to the analyte co-located with the first reactant. Further, if a first reactant is too close to an analyte, the first reactant may become partially- or fully-occluded (e.g., sterically, electrostatically, etc.) from reacting with a second reactant. Alternatively, partial occlusion of a first reactant may facilitate kinetic control of the reaction rate of the first reactant. Accordingly, a separation distance between a first reactant and an analyte at an address may be an optically non-resolvable distance, such as no more than about 500 nm, 400 nm, 300 nm, 200 nm, 100 nm, 50 nm, 40 nm, 30 nm, 20 nm, 15 nm, 10 nm, 5 nm, 1 nm, or less than 1 nm. Alternatively or additionally, a separation distance between a first reactant and an analyte at an address may be at least about 1 nanometer (nm), 5 nm, 10 nm, 15 nm, 20 nm, 30 nm, 40 nm, 50 nm, 100 nm, 200 nm, 300 nm, 400 nm, 500 nm, or more than 500 nm.

Further provided herein is a probe composition, comprising: (a) an affinity reagent having a binding specificity for at least a first analyte, (b) a first reactant, wherein the first reactant has reactivity with a second reactant, and (c) a linker, wherein the linker couples the affinity reagent to the first reactant, wherein the linker permits the first reactant to react with the second reactant when the affinity reagent is bound to the analyte. In some cases, the length of a linker coupling an affinity reagent to a first reactant may be greater than a separation distance between an analyte and a second reactant that is co-localized with the analyte. In other cases, the length of a linker coupling an affinity reagent to a first reactant may be less than a separation distance between an analyte and a second reactant that is co-localized with the analyte. The linker length may be a useful parameter for controlling binding equilibrium and binding kinetics according to the methods set forth herein.

The present disclosure further provides binding assays that record the presence of binding interactions, facilitating detection of such binding interactions even if the binding interaction becomes dissociated. In some configurations, a binding interaction may be directly recorded by binding together a molecular complex between bound species for a sufficiently long period of time to detect the complex. In other configurations, a binding interaction may be indirectly recorded by providing a detectable label at an address that can be subsequently detected at the address after a molecular complex between bound species has been disrupted.

Methods set forth herein may be particularly useful for characterizing pluralities of analytes, in which the plurality of analytes is characterized by a measure of heterogeneity. Heterogeneity of a plurality of analytes may be characterized by the presence of chemical or structural diversity amongst the analytes. For example, a plurality of proteins may contain two or more species of proteins, in which the species of proteins are distinguished from each other by differing primary structures (e.g., a first species having a different amino acid sequence than a second species). Additionally or alternatively, a plurality of proteins may contain two or more species of proteins, in which the species of proteins are distinguished from each other by differing proteoforms (e.g., a first species having a different complement of post-translational modifications than a second species). Additionally or alternatively, a plurality of proteins may contain two or more species of proteins, in which the species of proteins are distinguished from each other by differing isoforms (e.g., a first species having a different amino acid sequence splicing than a second species). A method set forth herein may be useful for characterizing a heterogeneous plurality of analytes that contains at least about 1%, 5%, 10%, 20%, 25%, 30%, 40%, 50%, 60%, 70%, 75%, 80%, 90%, 95%, 99%, or more than 99% of the species diversity of a genome, transcriptome, and/or proteome.

A plurality of analytes may comprise at least about 1, 2, 5, 10, 50, 100, 200, 500, 1000, 2000, 5000, 10000, 20000, 30000, 50000, 100000, 1000000, or more than 1000000 different species of analytes. Alternatively or additionally, a plurality of analytes may comprise no more than about 1000000, 100000, 50000, 30000, 20000, 10000, 5000, 2000, 1000, 500, 200, 100, 50, 10, 5, 2, or less than 5 different species of analytes.

Methods set forth herein may utilize one or more affinity reagents. It may be particularly useful to utilize an affinity reagent that binds to two or more differing species of analytes that are present in a plurality of analytes. In some cases, an affinity reagent may bind to an epitope that is present in two or more differing species of analytes that are present in a plurality of analytes. For example, an affinity agent may be configured to bind to an epitope (e.g., a trimer or tetramer amino acid sequence) that is common to two or more differing species of analytes.

An affinity reagent may bind to two or more differing binding targets with differing binding characteristics. For example, an affinity reagent may bind to a first binding target (e.g., a first analyte, a first epitope common to two or more differing analytes, etc.) with a first dissociation constant, and may further bind to a second binding target (e.g., a second analyte, a second epitope common to two or more differing analytes, etc.) with a second dissociation constant, in which the first dissociation constant differs from the second dissociation constant. Thus, the affinity reagent may dissociate, or separate, from one of the first binding target or the second binding target at a higher propensity than the other binding target. Useful distinguishing binding characteristics can also include the binding on-rate and/or the binding off-rate. Accordingly, for a plurality of analytes containing a first binding target and a second binding target for an affinity agent, there may exist two or more separate binding equilibria for the affinity agent. Some methods set forth herein may be useful for distinguishing between differing binding targets with a common affinity to an affinity reagent based upon the differences in binding equilibrium.

For affinity reagents described herein, a binding interaction with a binding target or plurality thereof having a highest characterized binding specificity (e.g., as determined by a lowest value of the affinity reagent's dissociation constant) may be referred to as a superordinate binding interaction. Likewise, for affinity reagents described herein, a binding interaction with a binding target or plurality thereof having a lower characterized binding specificity (e.g., as determined by a higher value of the affinity reagent's dissociation constant) relative to a superordinate binding interaction may be referred to as a subordinate binding interaction. An affinity reagent may have two or more superordinate binding interactions. An affinity reagent may have two or more subordinate binding interactions.

Binding equilibrium may depend in part on the relative concentrations of unbound affinity reagents and/or unbound analytes. For example, increasing the concentration of unbound affinity reagents may tend to increase the quantity of target analytes bound by the affinity reagents. Dissociation constant is a frequently used characterization of an affinity reagent for a binding target that can be utilized to estimate the steady-state fraction of binding targets that will be bound by an affinity reagent at binding equilibrium. A method set forth herein may comprise forming a binding equilibrium between affinity reagents and a plurality of binding targets (e.g., two or more species of analytes, analytes comprising a common epitope, etc.), in which at least about 10%, 20%, 30%, 40%, 50%, 60%, 70%, 80%, 90%, 95%, 99%, or more than 99% of binding targets are bound by an affinity reagent at equilibrium. Alternatively or additionally, a method set forth herein may comprise forming a binding equilibrium between affinity reagents and a plurality of binding targets, in which no more than about 99%, 95%, 90%, 80%, 70%, 60%, 50%, 40%, 30%, 20%, 10%, or less than 10% of binding targets are bound by an affinity reagent at equilibrium. Some methods set forth herein may comprise a step of recording a binding interaction between an affinity reagent and a binding target such that the binding interaction can be detected even after the affinity reagent has dissociated from the binding target. Accordingly, a method may comprise detecting a binding interaction for a percentage of binding targets (e.g., two or more species of analytes, analytes comprising a common epitope, etc.) that exceeds a percentage of binding targets bound by the affinity reagent at the steady-state binding equilibrium.

An affinity reagent may bind to at least about 1, 2, 5, 10, 50, 100, 200, 500, 1000, 2000, 5000, 10000, 20000, 30000, 50000, 100000, 1000000, or more than 1000000 different species of analytes present in a plurality of analytes. Alternatively or additionally, an affinity agent may bind to no more than about 1000000, 100000, 50000, 30000, 20000, 10000, 5000, 2000, 1000, 500, 200, 100, 50, 10, 5, or less than 5 different species of analytes present in a plurality of analytes.

Binding of unlabeled affinity reagents may be detected by a secondary detection moiety. A secondary detection moiety can comprise: (i) any affinity reagent or other molecule that has a binding specificity for at least a portion of a primary affinity reagent (i.e., an affinity reagent that binds an analyte target) and preferably does not have a binding specificity for an analyte, and (ii) a detectable label, as set forth herein. Preferably, a secondary detection moiety binds to a portion of an affinity reagent that does not facilitate binding of the affinity reagent to a target analyte. For example, a secondary detection moiety may bind to the Fc portion or an epitope thereof of an antibody, and preferably not bind to the variable regions of the antibody. Secondary detection moieties can include, for example, antibodies or fragments thereof that bind conserved portions of other antibody molecules. Secondary antibodies and secondary antibody fragments are commercially available for common forms of immunoglobulins, such as IgG. Further, secondary antibodies can be raised against libraries of affinity reagents to identify secondary affinity reagents that broadly bind a diverse library of affinity reagents. Other useful secondary detection moieties can include antibody-binding proteins such as Protein A, Protein G, Protein L, Protein M, or Protein A/G. In some cases, an affinity reagent can be attached to a peptide or protein that can be bound by a secondary detection moiety that is specific to the peptide or protein rather than the affinity reagent. For example, an antibody can be attached to a peptide tag such as a FLAG tag, an HA tag, a Myc-tag, a V5 tag, an E tag, an S tag, a His-tag, or an Avi-tag. Accordingly, the tagged antibody can be detected by a secondary detection moiety that has a binding specificity for the tag. Affinity reagents can be modified in a random, stochastic, or site-directed manner when attaching a tag moiety.

A secondary detection moiety may be an advantageous composition for attaching detectable labels to an affinity reagent. A secondary detection moiety may forego the added expense and challenges of attaching detectable labels to an affinity reagent without inhibiting the ability of the affinity reagent to bind an analyte. A secondary detection moiety can be selected to bind to a portion of the an affinity reagent that does not participate in the binding interaction between the affinity reagent and a target analyte. A detectable probe comprising a secondary detection moiety may also be smaller and/or have preferable mass transfer characteristics compared to a detectable probe using other macromolecular structures for attaching detectable labels (e.g., nucleic acid origami or other nucleic acid nanoparticles). For systems controlled by mass transfer limitations (e.g., systems utilizing analytes immobilized in wells), detectable probes comprising secondary detection moieties may be advantageous for reducing the necessary time to transfer detectable probes to target analytes.

In an aspect, provided herein is a method, comprising: (a) delivering a fluidic medium from a first vessel to a second vessel, wherein the fluidic medium comprises a plurality of detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe comprises: (i) an affinity reagent comprising a binding region and a non-binding region, (ii) a molecule non-covalently bound to the non-binding region of the affinity reagent, and (iii) a detectable label attached to the molecule, (b) binding detectable probes of the plurality of detectable probes to analytes of the plurality of analytes, and (c) detecting at single-analyte resolution for each site of the plurality of sites a presence or absence of a signal from the detectable label of a detectable probe of the plurality of detectable probes.

In another aspect, provided herein is a method, comprising: (a) delivering a fluidic medium from a first vessel to a second vessel, wherein the fluidic medium comprises a plurality of first detectable probes and a plurality of second detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe of the plurality of first detectable probes and the plurality of second detectable probes comprises: (i) an affinity reagent comprising a binding region and a non-binding region, (ii) a molecule non-covalently bound to the non-binding region of the affinity reagent, and (iii) a detectable label attached to the molecule, (b) binding detectable probes of the plurality of first detectable probes and plurality of second detectable probes to analytes of the plurality of analytes, and (c) detecting at single-analyte resolution for each site of the plurality of sites a presence or absence of a signal from the detectable label of a detectable probe of the plurality of first detectable probes and the detectable label of a detectable probe of the plurality of second detectable probes, wherein the non-binding region of the affinity reagents of the detectable probes of the plurality of first detectable probes differ from the non-binding region of the affinity reagents of the detectable probes of the plurality of second detectable probes, and wherein the detectable labels of the detectable probes of the plurality of first detectable probes differ from the detectable labels of the detectable probes of the plurality of second detectable probes.

In another aspect, provided herein is a system, comprising: (a) a first vessel, wherein the first vessel comprises a fluidic medium, wherein the fluidic medium comprises a plurality of detectable probes, wherein each detectable probe comprises: (i) an affinity reagent comprising a binding region and a non-binding region, (ii) a molecule non-covalently bound to the non-binding region of the affinity reagent, and (iii) a detectable label attached to the molecule, (b) a second vessel comprising a solid support, wherein the solid support comprises a plurality of sites, wherein a plurality of analytes is immobilized on the plurality of sites, wherein each site of the plurality of sites contains only one analyte of the plurality of analytes, (c) a fluid transfer device, as set forth herein, wherein the fluid transfer device provides fluidic communication between the first vessel and the second vessel, (d) an optical detector, as set forth herein, and (e) one or more processors, as set forth herein, programmed to read data from the optical detector to detect presence or absence of a signal from the detectable label at each site of the plurality of sites at single-analyte resolution.

A secondary detection moiety may be attached to a detectable label or a plurality thereof. Methods for attaching detectable labels to certain secondary detection moieties, such as secondary antibodies and secondary antibody fragments, are known in the art. The degree of labeling of a secondary detection moiety may depend upon the signal intensity requirements of an optical detection system. A secondary detection moiety can be attached to at least about 1, 2, 3, 4, 5, 6, 7, 8, 9, 10, 15, 20, 25, 30, 40, 50, or more than 50 detectable labels. Alternatively or additionally, a secondary detection moiety can be attached to no more than about 50, 40, 30, 25, 20, 15, 10, 9, 8, 7, 6, 5, 4, 3, 2 or less than 2 detectable labels.

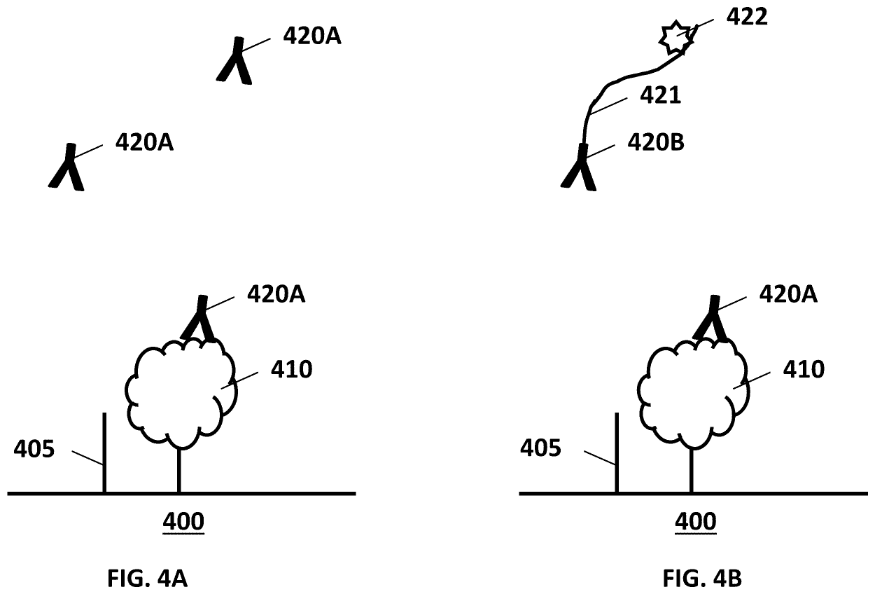

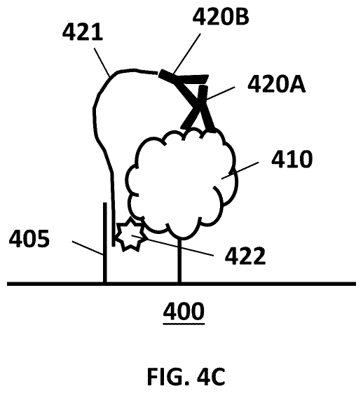

FIGS. 4A-4C illustrate steps of a method that utilizes a secondary antibody to detect the binding interaction of a primary antibody with an analyte. FIG. 4A depicts an address of a solid support 400, in which the address contains a co-localized analyte 410 with a complementary oligonucleotides 405. The solid support 400 is contacted with a plurality of unmodified primary affinity reagents 420A. A primary affinity reagent 420A has bound to the analyte 410 at the address of the solid support 400. FIG. 4B depicts a second configuration after unbound primary affinity reagents 420A have been rinsed from contact with the solid support 400. The rinsing medium may contain a secondary affinity reagent 420B, or a medium comprising the secondary affinity reagent 420B may be provided after the rinsing step. As shown in FIG. 4B, the secondary affinity reagent 420B is provided as a modified binding reagent, further comprising an attached linking moiety 421 and a detectable label 422. The secondary affinity reagent 420B has a binding specificity for a portion of the primary affinity reagent 420A molecule. FIG. 4C depicts a third configuration, in which the secondary affinity reagent 420B has bound to the primary affinity reagent 420A. In some configurations, the linking moiety 421 of the secondary affinity reagent 420B complex can attach to the complementary oligonucleotide 405, thereby co-localizing the secondary affinity reagent 420B at the address of the solid support 400. Subsequently, a signal from the detectable label 422 attached to the secondary affinity reagent 420B can be detected at the address of the solid support 400 containing analyte 410. It will be recognized that numerous systems and methods set forth herein can be readily modified to include detection by a secondary affinity reagent. It will be further recognized that a secondary affinity reagent may be provided as any affinity reagent composition set forth herein (e.g., attached to a tether and/or retaining component, configured for attachment to a bridging moiety, attached to a detectable label that is configured for FRET-based detection, attached to an enzymatic recording system, etc.).

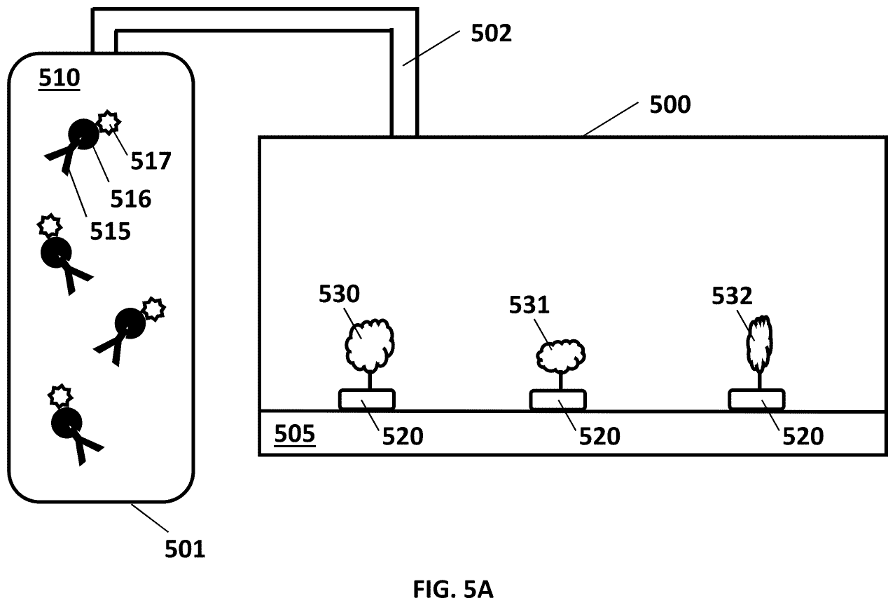

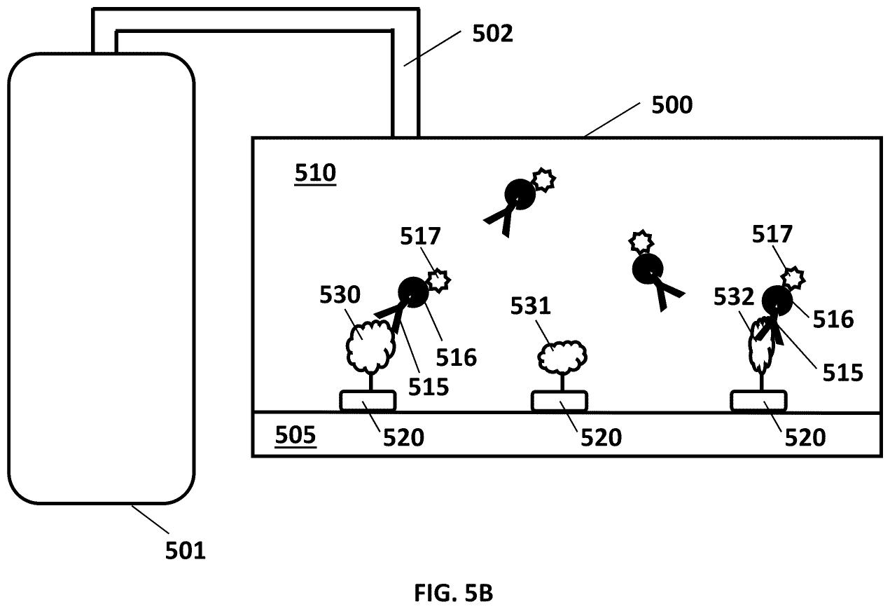

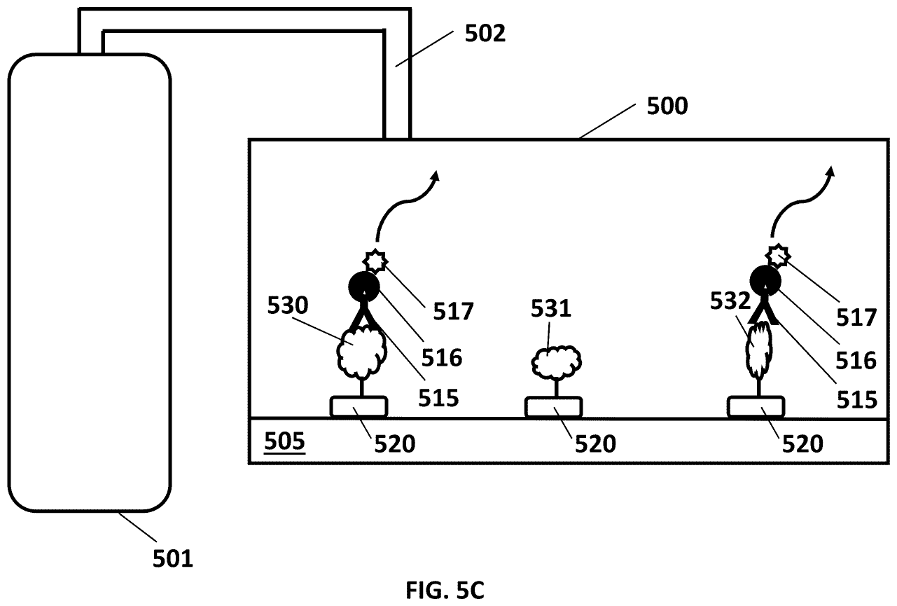

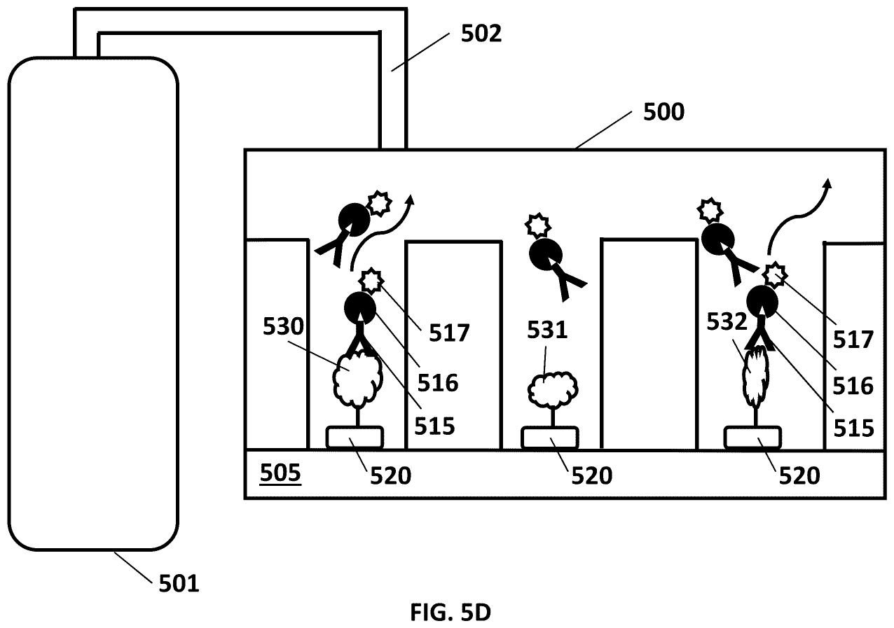

In some cases, a secondary detection moiety may be associated to an affinity reagent before the affinity reagent is contacted with an analyte. FIGS. 5A-5D depict usage of detectable probes for analyte detection, in which each detectable probe comprises a secondary detection moiety 516 pre-associated to an affinity reagent 515. FIG. 5A depicts a fluidic device 500 that is in fluidic communication with a vessel 501 via a fluid transfer system 502. The vessel 501 contains a fluidic medium 510 comprising a plurality of detectable probes. Each detectable probe individually comprises an affinity reagent 515 associated to a secondary detection moiety 516 and a detectable label 517. A plurality of analytes (530, 531, 532) are attached to a surface of a solid support 505 in the fluidic device 500. Each of the analytes is attached to a particle 520 that is coupled to the surface of the solid support 505. FIG. 5B depicts the system after the fluidic medium 510 comprising the detectable probes has been transferred by the fluid transfer system 502 into the lumen of the fluidic device 500. Detectable probes have associated to analytes 530 and 532 by binding of affinity reagents 515 to the respective analytes. Other detectable probes remain unbound in the lumen of the fluidic device 500. FIG. 5C depicts detection of signals from the detectable labels 517 at the addresses containing analytes 530 and 532. Preferably, the signals are detected by a device that is configured for detection at single-analyte resolution, as set forth herein. Optionally, the unbound detectable probes depicted in FIG. 5B have been removed from the fluidic device 500 before the detection step of FIG. 5C. FIG. 5D depicts a useful alternative configuration of FIG. 5C. The fluidic device 500 contains a plurality of wells, with analytes 530, 531, and 532 immobilized via attachment of particles to the bottoms of the respective wells. Use of wells may be useful for detecting optical signals emitted only in the near vicinity of well bottoms, thereby facilitating detection of detectable probes bound to analytes in the presence of unbound detectable probes. For example, the unbound detectable probe present in the central well of FIG. 5D may not produce a detectable signal because it is not co-localized by binding to an analyte in the bottom of the well.

Alternatively, a secondary detection moiety can be delivered to a plurality of affinity reagents after the affinity reagents have bound to target analytes. In some cases, it may be preferable to remove unbound affinity reagents from contact with analytes before delivering secondary detection moieties to the affinity reagents and/or analytes.

Secondary detection moieties may be provided in excess relative to an affinity reagent to which the secondary detection moiety is configured to bind. A molar excess of secondary detection moieties can drive the equilibrium between primary affinity reagents and secondary detection moieties toward the bound state, thereby facilitating increased detection of the affinity reagents when bound to an analyte. Accordingly, if secondary detection moieties are pre-associated to primary affinity reagents to form detectable probes before the detectable probes are delivered to a plurality of analytes, a fluidic medium containing the detectable probes may contain a mixture of detectable probes (i.e., primary affinity reagents attached to secondary detection moieties), unbound secondary detection moieties, and unbound primary affinity reagents. The concentration of unbound secondary detection moieties may exceed the concentration of unbound primary affinity reagents in a fluidic medium containing pre-associated detectable probes.

Secondary detection moieties may be provided to a method, system, or composition in a molar excess relative to primary affinity reagents to which the secondary detection moieties are configured to bind. Secondary detection moieties may be provided to a method, system, or composition in a molar ratio of at least about 1.1:1, 1.2:1, 1.5:1, 2:1, 3:1, 4:1 5:1, 10:1, 20:1, 50:1, 100:1 or more than 100:1 relative to primary affinity reagents. Alternatively or additionally, secondary detection moieties may be provided to a method, system, or composition in a molar ratio of no more than about 100:1, 50:1, 20:1, 10:1, 5:1, 4:1 3:1, 2:1, 1.5:1, 1.2:1, 1.1:1 or less than 1.1:1 relative to primary affinity reagents.

Detectable probes comprising secondary detection moieties, as set forth herein, can be readily multiplexed, thereby facilitating detection of two or more target epitopes or analytes during a single detection cycle. Preferably, a multiplexed detectable probe composition may comprise a first affinity reagent and a second affinity reagent, in which a non-binding region of the first affinity reagent differs from a non-binding region of the second affinity reagent. For example, a multiplexed detectable probe mixture may comprise a mixture of human IgG antibodies and non-human IgG antibodies (e.g., rabbit IgG, goat IgG, etc.). Structural differences in non-binding regions of differing affinity reagents can provide binding orthogonality for secondary detection moieties, thereby ensuring that the differing affinity reagents are properly labeled. Multiplexed mixtures of differing affinity reagents can be labeled before delivery to analytes by combining the differing affinity reagents with two or more differing secondary detection moieties, each of the two or more secondary detection moieties having a binding specificity for only one of the differing affinity reagents. Accordingly, differing secondary detection moieties may be provided differing detectable labels to facilitate distinguished detection of each respective detectable probe. Alternatively, a multiplexed mixture of unlabeled differing affinity reagents can be bound to analytes, then differing secondary detection moieties can be delivered to the bound differing affinity reagents. If each differing secondary detection moiety is delivered individually then detected, each differing secondary detection moiety can be provided a same detectable label. Different detectable probes can be recognized by differences in detected signals between cycles of labeling and detection. Alternatively, a mixture of differing secondary detection moieties can be delivered to a plurality of bound differing affinity reagents, in which the differing secondary detection moieties differ with respect to binding specificities and detectable labels.

A detectable probe comprising a secondary detection moiety may be particularly useful for methods utilizing solid supports comprising wells. A secondary detection moiety may provide a sufficient quantity of detectable labels to a detectable probe while having improved mass transfer characteristics relative to a detectable probe comprising a larger retaining component such as a nucleic acid particle. Accordingly, a detectable probe comprising a secondary detection moiety may be well suited to more rapidly diffuse into a well containing an immobilized analyte.

Certain methods set forth herein may include serial or cyclical detection with detectable probes. A detection sequence or cycle can include one or more steps of: (i) delivery of detectable probes to a plurality of analytes, (ii) incubation of the detectable probes with the plurality of analytes, (iii) optional removal of unbound detectable probes from contact with the plurality of analytes, (iv) detection of presence or absence of co-localization of a detectable probe with each analyte, and (v) dissociation of bound detectable probes from analytes. In some cases, a first sequence or cycle may utilize a first plurality of detectable probes and a second sequence or cycle may utilize a second plurality of detectable probes, in which the first plurality of detectable probes differs from the second plurality of detectable probes. In such cases, detectable probes may be removed from contact with a plurality of analytes after dissociation of bound probe-analyte complexes. In other cases, a first sequence or cycle may utilize a first plurality of detectable probes and a second sequence or cycle may utilize a second plurality of detectable probes, in which the first plurality of detectable probes is substantially identical to the second plurality of detectable probes. For example, a plurality of detectable probes can be recycled, or two different but substantially indistinguishable pluralities of detectable probes can be used. In such cases, detectable probes may optionally not be removed from contact with a plurality of analytes after dissociation of bound probe-analyte complexes. For example, heating of probe-analyte complexes may be sufficient to cause dissociation of probes from analytes, then subsequent cooling can facilitate re-association of detectable probes to analytes.

An advantage of repeated cycles of detection with a same type of detectable probe is the possibility of detecting probe-analyte interactions not observed in an initial detection sequence or cycle. For example, a first detection sequence may produce observation of detectable probe binding to a first set of analytes and a second detection sequence may produce observation of detectable probe binding to a second set of analytes, in which the first set of analytes and the second set of analytes differ.

Methods set forth herein may be particularly well-suited to array-based techniques. In some cases, a method may utilize a single-analyte array, in which each individual analyte of a plurality of analytes is co-localized with a unique identifier, such that each individual analyte is identifiable by its unique identifier. In some cases, a unique identifier may comprise an address on a solid support, in which the address is a spatially resolvable distance from any other address of the solid support that contains an analyte. In some cases, an address on a solid support may contain a site that is configured to bind an analyte. Accordingly, the site may comprise one or more moieties that facilitate attachment of the analyte and/or unique identifier co-localized with the analyte. In some cases, a unique identifier may comprise a barcode (e.g., a nucleic acid barcode, a peptide barcode, etc.) or a tag (e.g., a nucleic acid tag, a peptide tag) that is co-localized with an analyte. A tag may be especially useful for methods that comprise a step of recording a binding interaction of an affinity agent to an analyte via transfer of a detectable label to a tag co-localized with the analyte.

A single-analyte array can comprise a plurality of sites, with each site separated from all other sites by an optically resolvable distance. In some configurations, a single-analyte array may comprise a plurality of wells (e.g., a zero-mode waveguide array), in which each well contains a single site. Accordingly, each well of a single-analyte array may be configured to immobilize a single analyte within the well.

### Methods of Detecting Binding Interactions

In an aspect, provided herein is a method, comprising: (a) contacting a plurality of affinity reagents to a plurality of analytes for an equilibration period, in which the equilibration period is sufficient to form a binding equilibrium between affinity reagents of the plurality of affinity reagents and analytes of the plurality of analytes, and (b) after the equilibration period, detecting complexes comprising an affinity reagent bound to an analyte, in which the quantity of detected complexes is greater than a steady-state quantity of complexes that exists at the binding equilibrium.

In another aspect, provided herein is a method, comprising: (a) contacting a plurality of affinity reagents to a plurality of analytes for an equilibration period, in which the equilibration period is sufficient to form a binding equilibrium between affinity reagents of the plurality of affinity reagents and analytes of the plurality of analytes, and (b) after the equilibration period, detecting which analytes of the plurality of analytes formed a binding interaction with an affinity reagent of the plurality of affinity reagents during the equilibration period, in which the quantity of binding interactions is greater than a steady-state quantity of binding interactions that exists at the binding equilibrium.

In another aspect, provided herein is a method, comprising: (a) contacting a plurality of affinity reagents to a plurality of analytes for an equilibration period, in which each analyte of the plurality of analytes is individually paired to a unique identifier, (b) during the equilibration period, recording binding interactions at unique identifiers containing an affinity reagent bound to an analyte, and (c) detecting presence of absence of signals at a subset of the unique identifiers, thereby identifying analytes bound by affinity reagents during the equilibration period at the subset of unique identifiers.

In another aspect, provided herein is a method, comprising: (a) contacting a plurality of affinity reagents to a plurality of analytes for an equilibration period, thereby coupling affinity reagents of the plurality of affinity reagents to analytes of the plurality of analytes, wherein each analyte of the plurality of analytes is paired to a unique identifier, (b) forming a detectable reaction for analytes bound by an affinity reagent during the equilibration period, and (c) detecting presence of the detectable reaction at a subset of unique identifiers, thereby detecting the subset of analytes of the plurality of analytes bound by an affinity reagent of the plurality of affinity reagents.

Methods set forth herein may contain the steps of: (i) forming a binding interaction between an affinity reagent and an analyte, and (ii) forming a reaction between a first moiety and a second moiety, in which the first moiety is co-localized with the affinity reagent and the second moiety is co-localized with the analyte. A moiety may be co-localized with an affinity reagent or analyte if the moiety is attached to the affinity reagent or analyte, or if the moiety and affinity reagent or analyte are each attached to a solid support, molecule, or particle at a separation distance such that the moiety and the affinity reagent or analyte are not optically resolvable from each other. Preferably, a moiety co-localized with a first affinity reagent or analyte may not be optically resolvable from the affinity reagent or analyte, but will be optically resolvable from a moiety co-localized with a second affinity reagent or analyte.

A moiety may be co-localized with an analyte if a separation distance between the moiety and the analyte is no more than about 100 nanometers (nm), 80 nm, 60 nm, 50 nm, 40 nm, 30 nm, 20 nm, 10 nm, 5 nm, 1 nm, or less than 1 nm. Alternatively or additionally, a moiety may be co-localized with an analyte if a separation distance between the moiety and the analyte is at least about 1 nm, 5 nm, 10 nm, 20 nm, 30 nm, 40 nm, 50 nm, 60 nm, 80 nm, 100 nm, or more than 100 nm.

A reaction may be any suitable interaction between a first moiety and a second moiety. A reaction may be characterized by at least one of: (i) inhibiting dissociation of an affinity reagent from an analyte due to the interaction of the first moiety with the second moiety, and (ii) facilitating transfer of a detectable label associated with a binding interaction between the affinity reagent and the analyte (e.g., transferring a detectable label to an analyte or a unique identifier co-localized with the analyte, transferring a detectable label from an analyte or a unique identifier co-localized with the analyte). Preferably, a reaction may co-localize a detectable label at an address containing an analyte or a unique identifier such that the detectable label remains detectable at the address in the presence of a condition that disrupts a binding equilibrium between an affinity reagent and the analyte (e.g., rinsing unbound affinity reagents from contact with the analyte), or in the presence of a condition that dissociates the binding interaction between the affinity reagent and the analyte (e.g., contacting with a denaturing species, altering a pH, altering an ionic strength, altering a temperature, etc.).

Affinity reagents may be contacted to analytes for an equilibration period, in which the equilibration period is sufficient for a binding equilibrium to occur between the affinity reagents and the analytes. A binding equilibrium can occur when the rate of affinity reagents associating to analytes is equal to the rate of affinity reagents dissociating from analytes. Accordingly, for fixed quantities of affinity reagents and analytes, the total quantity of complexes formed by affinity reagents associated to analytes will be substantially constant at binding equilibrium, although the specific associated or unassociated molecules may differ between two time points during the binding equilibrium. For affinity reagents contacted to immobilized analytes, binding equilibrium may be disrupted by removal of unbound affinity reagents. The decrease in concentration of affinity reagents around the analytes may facilitate dissociation of bound affinity reagents from analytes. Likewise, binding equilibrium may be altered by changes in the chemical environment surrounding the analytes and affinity reagents. Changes in pH, ionic strength, buffer composition, surfactant concentration, and temperature can increase or decrease the quantity of affinity reagent/analyte complexes occurring at steady state.

The amount of time necessary to achieve binding equilibrium can depend upon the binding characteristics of affinity reagents and analytes, as well as surrounding chemical environment. Binding on-rates and off-rates can vary depending upon the affinity reagent and its binding target. Methods of measuring binding characteristics of affinity reagents are known in the art, and the skilled person can readily determine a sufficient equilibration period to provide a binding equilibrium between affinity reagents and analytes. An equilibration period may occur for at least about 30 seconds(s), 1 minute (min), 2 mins, 3 mins, 4 mins, 5 mins, 10 mins, 15 mins, 30 mins, 60 mins, or more than 60 mins. Alternatively or additionally, an equilibration period may occur for no more than about 60 mins, 30 mins, 15 mins, 10 mins, 5 mins, 4 mins, 3 mins, 2 mins, 1 min, 30 s, or less than 30 s.

Some methods set forth herein may comprise a step of recording a binding interaction between an affinity reagent and an analyte. A binding interaction may be directly recorded if a reaction inhibits dissociation of the affinity reagent from the analyte, thereby facilitating detection of the complex comprising the affinity reagent and analyte (e.g., by detection of a detectable label co-localized with the affinity reagent or co-localized with a moiety that forms a reaction). A binding interaction may be indirectly recorded if a reaction transfers a detectable label between the affinity reagent and analyte, thereby providing a change in detectable signal that is detectable after the affinity reagent has dissociated from the analyte.

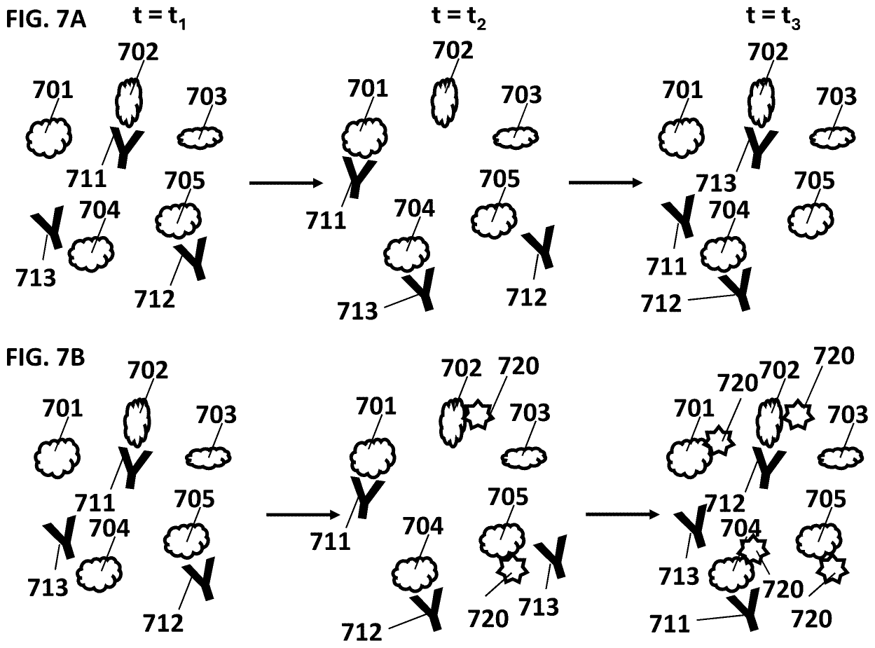

FIGS. 7A and 7B illustrate a potential advantage of recording binding interactions by methods set forth herein. Steady-state binding between a plurality of analytes (701, 702, 703, 704, and 705) and a plurality of affinity agents (711, 712, 713) is shown at three time points, t₁, t₂, and t₃, respectively. At time point t₁, affinity agent 711 is bound to analyte 702, affinity agent 712 is bound to analyte 705, and affinity agent 713 is unbound. At time point t₂, affinity agent 711 is bound to analyte 701, affinity agent 713 is bound to analyte 704, and affinity agent 712 is unbound. At time point t₃, affinity agent 712 is bound to analyte 704, affinity agent 713 is bound to analyte 702, and affinity agent 711 is unbound. Under a steady-state equilibrium condition, 40% of analytes are observed to be bound by an affinity reagent at any given time point. In scenario A (upper), detection of the system can provide the equilibrium bound fraction, but provides no information on which analytes formed binding interactions with affinity reagents at time points before or after the detection event. In scenario B (lower), each binding interaction between an analyte and an affinity reagent is recorded by attaching a detectable label 720 to the analyte. In scenario B, at time point t₃, a greater fraction of analytes can be detected to have participated in a binding interaction with an affinity agent than a fraction of analytes bound to affinity reagents at any given time point in the steady-state equilibrium condition. If analytes are provided in a spatially-resolvable format (e.g., an array of analytes), it may be possible to detect individually whether each analyte formed a binding interaction with an affinity reagent during an equilibration period by recording the binding interactions at unique identifiers co-localized with each individual analyte.

Accordingly, for binding interactions between a plurality of analytes and a plurality of affinity reagents, binding interactions between analytes and affinity reagents may be detected for a fraction of analytes that exceeds the steady-state fraction of analytes that are bound by the affinity reagents at equilibrium. The fraction of analytes of a plurality of analytes with a detected binding interaction may exceed the steady-state fraction of analytes of the plurality of analytes that are bound by the affinity reagents at equilibrium by at least about 0.1%, 0.5%, 1%, 2%, 3%, 4%, 5%, 6%, 7%, 8%, 9%, 10%, 15%, 20%, 30%, 40%, 50%, or more than 50%. For example, if the fraction of analytes of a plurality of analytes with a detected binding interaction exceeds the steady-state fraction of analytes that are bound by the affinity reagents at equilibrium by 5%, and the steady-state fraction of analytes of a plurality of analytes that are bound by the affinity reagents at equilibrium is 10%, the fraction of analytes with a detected binding interaction may be about 15% of the plurality of analytes. Alternatively or additionally, the fraction of analytes of a plurality of analytes with a detected binding interaction may exceed the steady-state fraction of analytes of the plurality of analytes that are bound by the affinity reagents at equilibrium by no more than about 50%, 40%, 30%, 20%, 15%, 10%, 9%, 8%, 7%, 6%, 5%, 4%, 3%, 2%, 1%, 0.5%, 0.1%, or less than 0.1%. In some cases, binding interactions between analytes and affinity reagents may be detected for a fraction of analytes that is substantially the same as or less than the steady-state fraction of analytes that are bound by the affinity reagents at equilibrium.

A method may comprise a step of removing unbound affinity reagents from contact with a plurality of analytes before detecting which analytes have formed a binding interaction with an affinity reagent during an equilibration period. Such a step may be useful if the binding interaction has been directly recorded. Alternatively, a method may comprise a step of dissociating complexes, each complex comprising an analyte associated to an affinity reagent, after contacting affinity reagents to analytes and before detecting which analytes have formed a complex with an affinity reagent during an equilibration period. Such a step may be useful if the binding interaction has been indirectly recorded. Alternatively, a method set forth herein may not comprise a step of removing unbound affinity reagents before detecting which analytes have formed a binding interaction with an affinity reagent during an equilibration period. Accordingly, detection of the binding interaction between affinity reagents and analytes may occur in the presence of unbound affinity reagents.

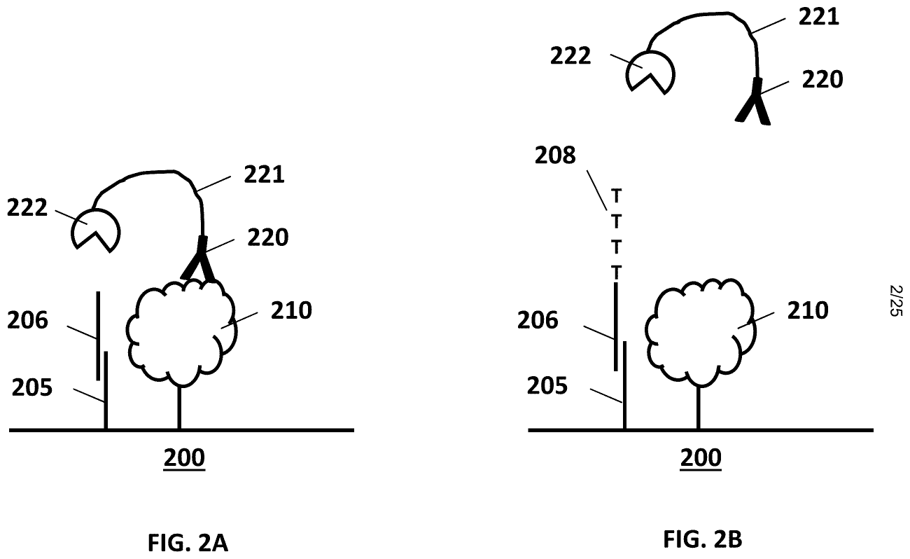

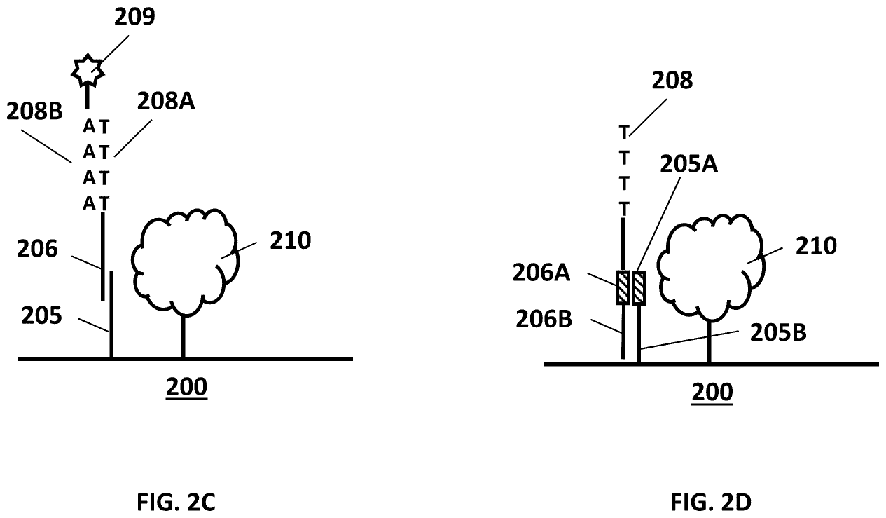

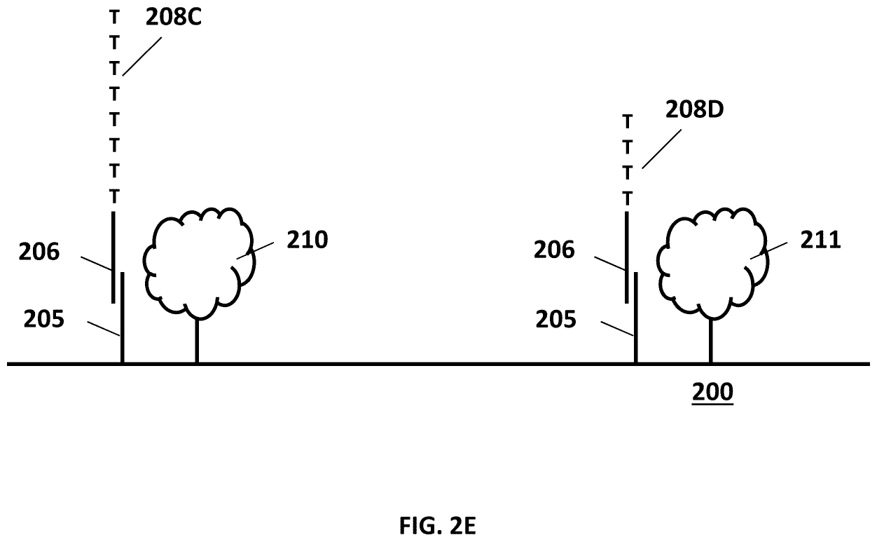

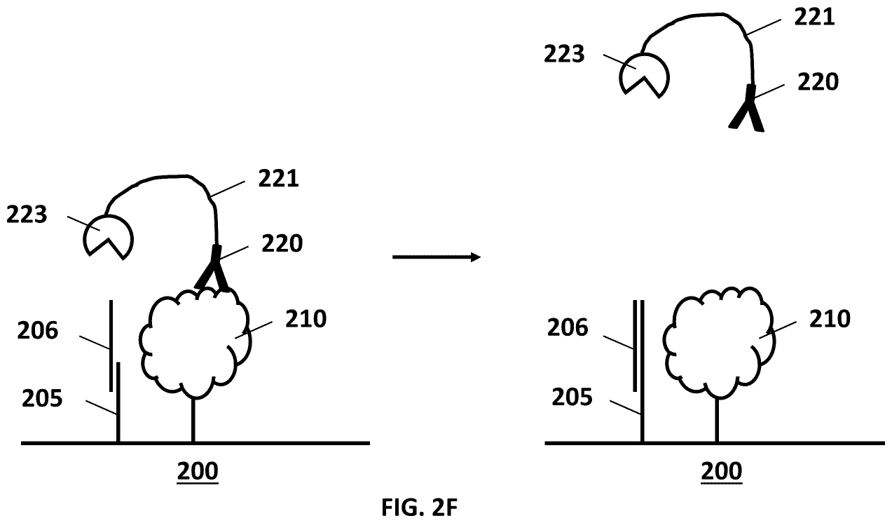

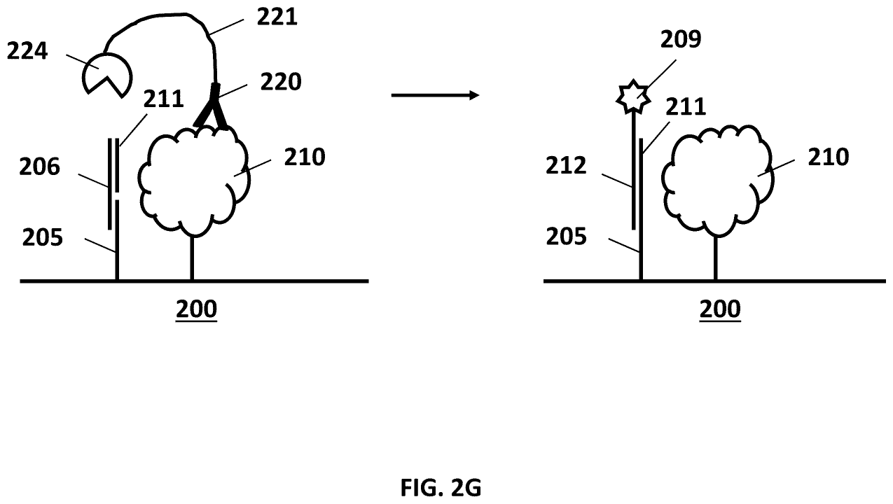

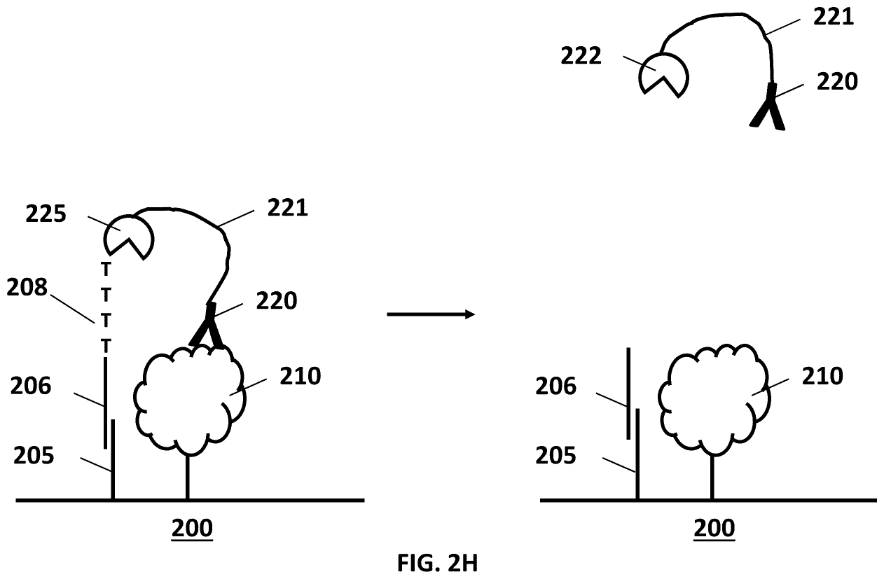

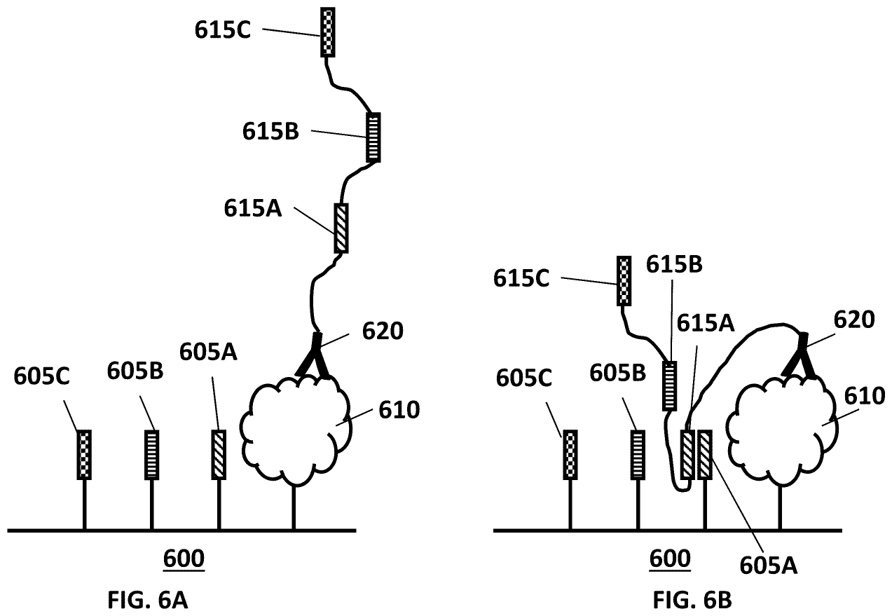

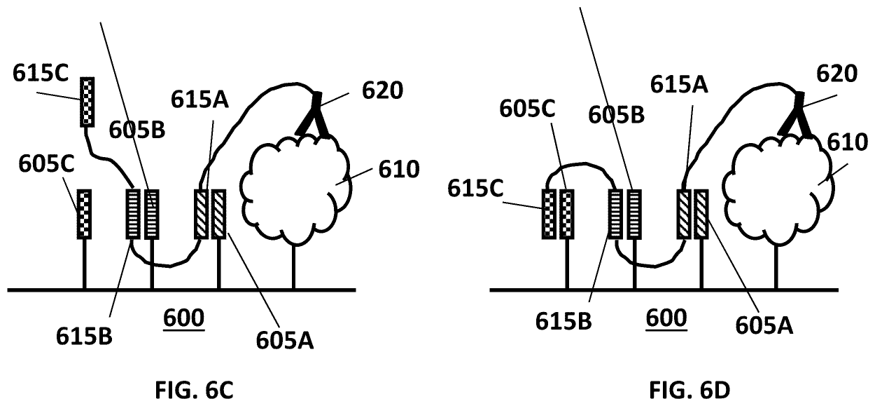

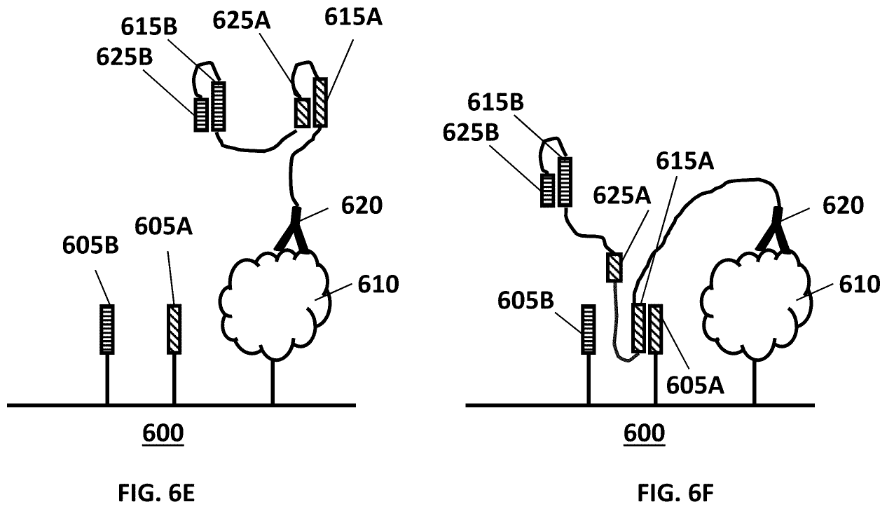

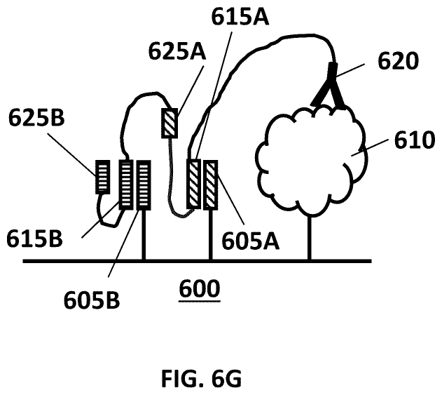

A method may comprise a step of recording a binding interaction between an affinity reagent and an analyte, in which recording the binding interaction between the affinity reagent and the analyte comprises forming a reaction between a first moiety co-localized with the affinity reagent and a second moiety co-localized with the analyte. In some cases, the reaction can inhibit dissociation of the affinity reagent from the complex. For example, methods of recording a binding interaction by inhibiting dissociation are shown in FIGS. 6A-6G. In other cases, the reaction can produce a detectable signal. For example, methods of recording a binding interaction that produces a detectable signal are shown in FIGS. 2A-2H, 4A-4C, 5A-5D, and 8A-8E.

In some methods, a binding interaction between an affinity reagent and an immobilized analyte may be directly recorded by forming a reaction that immobilizes the affinity reagent with the analyte. A kinetically-controlled binding reagent may be utilized to control the rate of formation of the reaction. Preferably, the on-rate or forward rate of the reaction is slower than the on-rate of the affinity reagent to the analyte, thereby reducing the likelihood of the reaction occurring before a binding interaction has occurred. In some cases, a reaction may bind a first moiety co-localized with the affinity reagent to a second moiety co-localized with the analyte. In such cases, a kinetically-controlled detection agent (e.g., an oligonucleotide comprising a secondary structure, a reactive functional group, a component of a ligand-receptor binding pair, etc.) may be incorporated into the first moiety, the second moiety, or both the first and second moiety.

In some methods, a binding interaction between an affinity reagent and an immobilized analyte may be directly recorded by forming a reaction that transfers a detectable label between the affinity reagent and the immobilized analyte or a unique identifier co-localized with the analyte). Kinetically-controlled indirect recording methods could include enzymatic labeling of nucleic acid or peptide tags, photo-catalyzed binding of detectable labels to analytes or unique identifiers co-localized therewith, or transfer of a transferrable moiety between an affinity reagent and an analyte or a unique identifier co-localized therewith. Oligonucleotide moieties comprising secondary structure may be useful transferrable moieties for controlling the rate of transfer of the transferrable moiety between an affinity reagent and an analyte or a unique identifier co-localized therewith.

In some cases, an affinity reagent may be contacted to an analyte in the presence of a kinetically-controlled detection agent. Such a configuration may be utilized if the on-rate or reaction rate of the kinetically-controlled detection agent is substantially slower than the on-rate of the affinity reagent to the analyte. Alternatively, a kinetically-controlled detection agent may be contacted to an analyte after contacting an affinity reagent to the analyte. Such a configuration may be utilized if the on-rate or reaction rate of the kinetically-controlled detection agent is substantially faster or equal to the on-rate of the affinity reagent to the analyte.

The on-rate or forward reaction rate of a kinetically-controlled detection agent may be chosen to distinguish superordinate binding interactions from subordinate binding interactions of an affinity reagent. For example, if a binding on-rate of a kinetically-controlled detection agent is slower than a binding off-rate of a subordinate binding interaction of the affinity agent, it is unlikely that the reaction facilitated by the kinetically-controlled detection agent will occur in the presence of the subordinate binding interaction. In another example, if a binding on-rate of a kinetically-controlled detection agent is faster than a binding off-rate of a superordinate binding interaction of the affinity agent, it is likely that the reaction facilitated by the kinetically-controlled detection agent will occur in the presence of the superordinate binding interaction. In some cases, a method may utilize two differing kinetically-controlled detection agents, in which the first kinetically-controlled detection agent has a slower on-rate than the off-rate of a subordinate binding interaction, in which the second kinetically-controlled detection agent has a faster on-rate than the off-rate of a subordinate binding interaction, and in which the first and second kinetically-controlled detection agents each have a faster on-rate than the off-rate of a superordinate binding interaction. In such cases, reactions facilitated by both kinetically-controlled binding reagents will only be detected for a superordinate binding interaction of an affinity reagent and an analyte, while only the reaction of the second kinetically-controlled detection agent will be detected for a subordinate binding interaction of the affinity reagent and an analyte. Many methods set forth herein may readily be modified to include two or more differing kinetically-controlled detection agents for identification of superordinate or subordinate binding interactions.

Binding kinetics of kinetically-controlled detection agents can readily be measured by various methods, including empirical measurement of binding or reaction rates, as well as in silico estimation of binding or reaction rates for certain types of kinetically-controlled detection agents (e.g., structured oligonucleotides, enzymes, etc.). Accordingly, measured binding kinetics of kinetically-controlled detection agents can be compared to binding kinetics of affinity reagents to identify suitable combinations for methods set forth herein.

In some cases, a kinetically-controlled detection agent can comprise a detectable label (e.g., a detectable label attached to or co-localized with the kinetically-controlled detection agent). For example, a detection reagent may comprise a tether strand comprising a first detectable label and an analyte may be co-localized with a docker strand comprising a second detectable label, in which the first detectable label and the second detectable can provide a Forster Resonance Energy Transfer (FRET) signal when the tether strand is coupled to the docker strand. Alternatively, a kinetically-controlled detection agent may not comprise a detectable label. For example, enzymatic detection agents may attach substrates to an analyte, an affinity agent, or a moiety co-localized therewith. In some cases, an affinity reagent, an analyte, or a unique identifier may comprise a detectable label.

The present disclosure further provides systems for measuring binding interactions by methods set forth herein. In another aspect, provided herein is a system, comprising: (a) a solid support comprising a plurality of sites, in which each site of the plurality of sites is optically resolvable from any other site of the plurality of sites, and (b) a plurality of analytes immobilized on the solid support, in which each site of the plurality of sites is attached to one and only one analyte of the quantity of analytes. in which, in a first configuration, the system further comprises a plurality of affinity reagents, in which a fraction of the affinity reagents is bound to analytes of the plurality of analytes, in which a quantity of the fraction of affinity reagents is determined by a binding equilibrium of the affinity reagents for the analytes, and in which, in a second configuration, the system is substantially devoid of affinity reagents, in which each individual site of a fraction of sites of the plurality of sites comprises a detectable label, in which a quantity of the fraction of sites is greater than a quantity of the second fraction of affinity reagents.

In another aspect, provided herein is a system, comprising: (a) a solid support comprising a plurality of sites, in which each site of the plurality of sites is optically resolvable from any other site of the plurality of sites, (b) a plurality of analytes immobilized on the solid support, in which each site of the plurality of sites is attached to one and only one analyte of the quantity of analytes, and (c) a plurality of affinity reagents bound analytes of the plurality of analytes at sites of the plurality of sites, in which the affinity reagents have a known binding equilibrium for binding to analytes of the plurality of analytes, and in which a quantity of the sites of the plurality of sites is greater than a quantity of analytes bound to affinity reagents based upon the known binding equilibrium.

Any of a variety of analytes can be used in a method or composition set forth herein. For ease of explanation, various methods and compositions will be exemplified herein in the context of using proteins. It will be understood that the exemplified methods and compositions can be extended to other analytes. Exemplary analytes include, but are not limited to, a tissue, cell, organelle, virus, nucleic acid (e.g., DNA or RNA), carbohydrate (e.g., monosaccharide, oligosaccharide or polysaccharide), glycan, vitamin, enzyme cofactor, hormone, or small molecule such as a candidate therapeutic agent, metabolite, nucleotide, nucleoside, amino acid, sugar, lipid, or the like. In some configurations, a composition or method set forth herein can lack one or more of the analytes set forth herein.

A composition or method set forth herein can be configured for a single analyte or for a plurality of different analytes. A plurality of analytes can include, for example, a proteome, or substantial fraction thereof, including a variety of different proteins; a genome, or substantial fraction thereof, including a variety of different DNA sequences; a transcriptome, or substantial fraction thereof, including a variety of different RNA sequences; a metabolome, or substantial fraction thereof, including a variety of different metabolites; or a microbiome, or substantial fraction thereof, including a variety of different microbes. These and other analytes known in the art can be used in compositions and methods set forth herein.

A protein or other analyte provided to a method set forth herein can be derived from a natural or synthetic source. Exemplary sources include, but are not limited to a biological tissue, fluid, cell or subcellular compartment such as an organelle (e.g., nucleus, mitochondria, chloroplast, endoplasmic reticulum, vesicle, cytoskeleton, vacuole, lysosome, cell membrane, cytosol or Golgi apparatus). For example, a sample can be derived from a tissue biopsy, biological fluid (e.g., blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or protein synthesis reaction. A primary source for a cancer biomarker protein may be a tumor biopsy sample. Other sources include environmental samples or forensic samples.

Exemplary organisms from which a protein or other analyte can be derived include, but are not limited to, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A protein can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A protein or other analyte can be derived from a homogeneous culture or population of the above organisms or alternatively from a collection of several different organisms, for example, in a community or ecosystem.

In some cases, a protein or other analyte can be derived from an organism that is collected from a host organism. A protein or other analyte may be derived from a parasitic, pathogenic, symbiotic, or latent organism collected from a host organism. A protein or other analyte can be derived from an organism, tissue, cell or biological fluid that is known or suspected of being associated with a disease state or disorder (e.g., an oncogenic virus). Alternatively, a protein or other analyte can be derived from an organism, tissue, cell or biological fluid that is known or suspected of not being associated with a particular disease state or disorder. For example, one or more proteins isolated from such a source can be used as a control for comparison to results acquired from a source that is known or suspected of being associated with the particular disease state or disorder. A sample may include a microbiome. A sample may include a plurality of proteins or other analytes of interest contributed by microbiome constituents. In some cases, one or more proteins (or other analytes) used in a method, composition or apparatus set forth herein may be obtained from a single organism (e.g., an individual human), single cell, single organelle, or single protein-containing particle (e.g., a viral particle).

In some cases, one or more proteins or other analytes of interest can be obtained from a single cell, protein-containing particle (e.g., a viral particle), or organelle. A single cell, protein-containing particle, or organelle may be collected by any known method in the art, such as fluorescence assisted cell sorting, magnetic-assisted cell sorting, and buoyancy-assisted cell sorting. In some cases, a single cell, protein-containing particle, or organelle may be collected by an emulsion technique such as liposome or micellar capture.

One or more analytes can optionally be separated or isolated from other components of the source for the analyte(s). For example, one or more proteins can be separated or isolated from lipids, nucleic acids, hormones, enzyme cofactors, vitamins, metabolites, microtubules, organelles (e.g., nucleus, mitochondria, chloroplast, endoplasmic reticulum, vesicle, cytoskeleton, vacuole, lysosome, cell membrane, cytosol or Golgi apparatus), other proteins or the like. Protein separation can be carried out using methods known in the art such as centrifugation (e.g., to separate membrane fractions from soluble fractions), density gradient centrifugation (e.g., to separate different types of organelles), precipitation, affinity capture, adsorption, liquid-liquid extraction, solid-phase extraction, chromatography (e.g., affinity chromatography, ion exchange chromatography, reverse phase chromatography, size exclusion chromatography, electrophoresis (e.g., polyacrylamide gel electrophoresis) or the like. Useful protein separation methods are set forth in Scopes, Protein Purification Principles and Practice, Springer; 3rd edition (1993).

A protein that is used in a composition or method set forth herein can be in a native or denatured conformation. For example, a protein can be in a native conformation, whereby it is capable of performing native function(s) such as catalysis of its natural substrate(s) or binding to its natural substrate(s). Alternatively, a protein can be in a denatured conformation whereby it is incapable of performing certain native function(s) such as catalysis of its natural substrate(s) or binding to its natural substrate(s). A protein can be in a native conformation for some manipulations set forth herein and in a denatured conformation for other manipulations set forth herein. A protein may be denatured at any stage during manipulation, including for example, upon removal from a native milieu or at a later stage of processing such as a stage where the protein is separated from other cellular components, fractionated from other proteins, functionalized to include a reactive moiety, attached to a particle or solid support, contacted with an affinity reagent, detected, or other manipulation. Any of a variety of denaturants can be used such as heat (e.g., temperatures greater than about 40° C., 60° C., 80° C. or higher), applied force such as magnetic force or fluidic force, excessive pH (e.g., pH lower than 4.0, 3.0 or 2.0; or pH greater than 10.0, 11.0 or 12.0); chaotropic agents (e.g., urea, guanidinium chloride, or sodium dodecyl sulfate), organic solvent (e.g., chloroform or ethanol), physical agitation (e.g., sonication) and/or radiation. A denatured protein may be refolded, for example, reverting to a native state for one or more steps of a process set forth herein.

An analyte, such as a protein, can be attached to a particle, solid support or other substance. An exemplary particle is a virus particle such as a phage. A particularly useful particle is a structured nucleic acid particle. Structured nucleic acid particles can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; US Pat. App. Pub. No. 2022/0162684 A1, or U.S. patent application Ser. No. 18/058,000, each of which is incorporated herein by reference.

A particle, such as a structured nucleic acid particle, may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to length, area (i.e., footprint), or volume. The size or shape of a particle can be smaller than an address in an array to which it will associate or attach. Optionally, the size or shape of particles in a population are configured to preclude more than one of the particles from occupying an address in an array.

Optionally, a particle (e.g., a structured nucleic acid particle) or population of particles can have a minimum, maximum or average length of at least about 10 nm, 25 nm, 50 nm, 100 nm, 250 nm, 500 nm, 1 micron, 5 microns, or more. Alternatively or additionally, a particle or population thereof can have a minimum, maximum or average length of no more than about 5 microns, 1 micron, 500 nm, 250 nm, 100 nm, 50 nm, 25 nm, 10 nm or less.

Optionally, a particle, such as a structured nucleic acid particle, or population thereof can have a minimum, maximum or average volume of at least about 1 micron³, 10 micron³, 100 micron³, 1 mm³ or more. Alternatively or additionally, a particle or population thereof can have a minimum, maximum or average volume of no more than about 1 mm³, 100 micron³, 10 micron³, 1 micron³ or less.

Optionally, the minimum, maximum or average area (i.e., footprint) for a particle, such as a structured nucleic acid particle, can be at least about 10 nm², 100 nm², 1 micron², 10 micron², 100 micron², 1 mm² or more. Alternatively or additionally, the minimum, maximum or average area for a particle footprint can be at most about 1 mm², 100 micron², 10 micron², 1 micron², 100 nm², 10 nm², or less. The footprint of a particle may have a regular shape or an approximately regular shape, such as triangular, square, rectangular, circular, ovoid, or polygonal shape.

An analyte can be attached to a label, for example, using compositions and methods set forth herein in the context of affinity reagents.

Optionally, an analyte of the present disclosure can be attached to a unique identifier in an array of unique identifiers. An array can include a number or variety of unique identifiers, for example, to accommodate a desired sample complexity. In some configurations, the array is configured for single-molecule resolution. For example, individual addresses of an array can each be attached to one, and only one, analyte. Alternatively, individual addresses of the array can each be attached to an ensemble of analytes. Several configurations for arrays and their methods of use will be exemplified below in the context of arrays having addresses as unique identifiers. It will be understood that the configurations can be extended to arrays having unique identifiers other than addresses.

The addresses of an array can optionally be optically observable and, in some configurations, adjacent addresses can be distinguishable when detected optically. Addresses of an array are typically discrete. The discrete addresses can be contiguous, or they can have interstitial spaces between each other. An array can have, for example, addresses that are separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more addresses (e.g., addresses to which at least one analyte is attached).

Arrays can be made using methods known in the art such as those that deposit analytes at predefined addresses on a surface or those that contact an array surface with a plurality of analytes in fluid phase such that the analytes are randomly distributed to addresses in the array. Exemplary arrays and methods for making and using arrays are set forth, for example, in U.S. Pat. Nos. 11,203,612 or 11,505,796; or US Pat. App. Pub. No. US 2023/0167488 A1, each of which is incorporated herein by reference.

An array can have a size and complexity that is sufficient to accommodate a plurality of proteins such as those exemplified below. Arrays of other analytes or arrays of affinity reagents can also have size and complexity exemplified below for proteins. It will be understood that the pluralities of proteins set forth below need not be limited to array configurations.

A plurality of proteins, whether present in an array or other composition set forth herein, can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins can include at least 1 pg, 10 pg, 100 pg, 1 ng, 10 ng, 100 ng, 1 ug, 10 ug, 100 ug, 1 mg, 10 mg, 100 mg or more protein by mass. Alternatively or additionally, a plurality of proteins may contain at most 100 mg, 10 mg, 1 mg, 100 ug, 10 ug, 1 ug, 100 ng, 10 ng, 1 ng, 100 pg, 10 pg, 1 pg or less protein by mass.

A plurality of proteins can include or be obtained from a proteomic sample. A proteomic sample can include substantially all proteins from a given source or a substantial fraction thereof. For example, a plurality of proteins may contain at least 60%, 75%, 90%, 95%, 99%, 99.9% or more of the total protein mass present in the source from which the sample was derived. Alternatively or additionally, a plurality of proteins may contain at most 99.9%, 99%, 95%, 90%, 75%, 60% or less of the total protein mass present in the source from which the sample was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein molecules, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length amino acid sequences in the plurality. For example, the variety of full-length amino acid sequences in a plurality of proteins can be equated with the number of different protein-encoding genes in the source for the plurality of proteins. Whether or not the proteins are derived from a known genome or from any genome at all, the variety of full-length amino acid sequences can be counted independent of presence or absence of post translational modifications in the proteins. A human proteome is estimated to have about 20,000 different protein-encoding genes such that a plurality of proteins derived from a human can include up to about 20,000 different full-length amino acid sequences. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity that includes substantially all different native-length amino acid sequences from a given source or a subfraction thereof. A proteome or subfraction can have a complexity of at least 2, 5, 10, 100, 1×10³, 1×10⁴, 2×10⁴, 3×10⁴ or more different native-length amino acid sequences. Alternatively or additionally, a proteome or subfraction can have a complexity that is at most 3×10⁴, 2×10⁴, 1×10⁴, 1×10³, 100, 10, 5, 2 or fewer different native-length amino acid sequences.

The diversity of a plurality of proteins can include at least one representative for substantially all proteins encoded by a source from which the plurality of proteins was derived or a substantial fraction thereof. For example, a plurality of proteins may contain at least one representative for at least 60%, 75%, 90%, 95%, 99%, 99.9% or more of the proteins encoded by a source from which the plurality of proteins was derived. Alternatively or additionally, a plurality of proteins may contain a representative for at most 99.9%, 99%, 95%, 90%, 75%, 60% or less of the proteins encoded by a source from which the plurality of proteins was derived.

A plurality of proteins can be characterized in terms of the variety of full-length amino acid sequences in the plurality including transcribed splice variants. The human proteome has been estimated to include about 70,000 different full-length amino acid sequences when splice variants are included. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different full-length amino acid sequences. Alternatively or additionally, a plurality of proteins can have a complexity that is at most 1×10⁶, 1×10⁵, 7×10⁴, 1×10⁴, 1×10³, 100, 10, 5, 2 or fewer different full-length amino acid sequences.

A plurality of proteins can be characterized in terms of the variety of protein structures therein including, for example, different full-length amino acid sequences or different proteoforms among those sequences. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the plurality. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A sample used herein, whether containing proteins or other analytes, need not be from a biological source and can instead be from an artificial source, such as a library from a combinatorial synthesis or a library from an in vitro synthesis that exploits biological components. An artificial sample can have a range of complexity similar to those set forth herein for proteomes. A method set forth herein can detect, identify or characterize some or all proteins in a proteome or other sample including, for example, at least about 1%, 5%, 10%, 25%, 50%, 75%, 90% or 99% of the proteins in the sample.

Any of a variety of affinity reagents can be used in a composition or method set forth herein. Particularly useful affinity reagents include, but are not limited to, antibodies whether full length or functional fragments thereof (e.g., Fab′ fragments, F(ab′)₂ fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, or functional fragments thereof. The exemplified affinity reagents can be used individually. Alternatively, an affinity reagent set forth herein can be used as a paratope or moiety of an affinity reagent having a plurality of paratopes or moieties. For example, an affinity reagent set forth herein can provide one paratope (or a subset of paratopes) of an affinity reagent having a plurality of paratopes. In some configurations, a composition or method set forth herein can lack one or more of the affinity reagents set forth herein.

An antibody is a particularly useful affinity reagent for use in a composition or method set forth herein. The antibody can be any antigen-binding molecule or molecular complex having at least one complementarity determining region (CDR) that binds to a particular epitope with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full-length and functional fragments can be particularly useful for many applications. The term “antibody” as used herein encompasses full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR1, CDR2 or CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may have any of a variety of sizes or amino acid compositions and will generally include at least one CDR which is adjacent to or in frame with one or more framework sequences. For antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} Or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-C_{H2}-C_{H}; (vi) V_{H}-C_{H2}-C_{H3}; (vi) V_{H}-C_{L}; (viii) V_{L}-C_{H1}; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., at least 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

An affinity reagent of the present disclosure can include one or more paratopes. For example, an affinity reagent can include at least 1, 2, 3, 4, 5, 10, 15, 20, 25 or more paratopes. Alternatively or additionally, an affinity reagent can include at most 25, 20, 15, 10, 5, 4, 3, 2, or 1 paratopes. Multiple paratopes that are present in an affinity reagent can have the same function or different functions compared to each other. For example, the multiple paratopes can each have affinity for the same epitope or same set of epitopes. In some cases, the strength and/or specificity of the affinity can be substantially the same, for example, in cases where the paratopes have the same structure. In other cases, the strength and/or specificity of two or more paratopes for a given epitope or set of epitopes can overlap despite some differences in the functional or structural characteristics of the two or more paratopes. In some configurations, multiple paratopes of a given affinity reagent can have affinity for different epitopes. This can be the case, whether the different epitopes are found in the same analyte (e.g., two different amino acid trimer epitopes present in a given protein analyte) or in different analytes (e.g., a first trimer epitope being found in a first protein that lacks a second trimer epitope, and a second trimer epitope being found in a second protein that lacks the first trimer epitope).

In some configurations of the methods, compositions or systems set forth herein, two or more affinity reagents can be present as moieties of a multimeric affinity reagent. For example, an affinity reagent can include two or more affinity moieties, wherein the affinity moieties are selected from an affinity reagent set forth herein or known in the art. Two or more affinity moieties can be combined via attachment to any of a variety of retaining components including, for example, a structured nucleic acid particle (SNAP), nucleic acid origami, artificial polymer or particle. Other particles (e.g., particles composed of solid support material set forth herein or known in the art) or substances, such as those set forth herein in the context of mediating attachment of a protein to a solid support, can be used as retaining components for affinity reagents. The presence of multiple affinity moieties in an affinity reagent can provide increased binding strength, for example, due to increased avidity as compared to any one of the affinity moieties when used as an individual affinity reagent. In some configurations an affinity reagent can include at least 2, 3, 4, 5, 6, 7, 8, 9, 10, 15, 20 or more affinity moieties. Alternatively or additionally, an affinity reagent can include at most 20, 15, 10, 9, 8, 7, 6, 5, 4, 3, 2 or fewer affinity moieties. It may be convenient to characterize an affinity reagent with respect to the number of paratopes it includes. For example, an affinity reagent can include at least 2, 3, 4, 5, 6, 7, 8, 9, 10, 15, 20 or more paratopes. Alternatively or additionally, an affinity reagent can include at most 20, 15, 10, 9, 8, 7, 6, 5, 4, 3, 2 or fewer paratopes. Typically, the affinity moieties or paratopes that are present in an affinity reagent will be structurally identical. For example, a plurality of antibodies in an affinity reagent can have identical amino acid sequences.

Whether or not a plurality of affinity moieties or a plurality of paratopes include structurally identical members, the members can recognize the same epitopes. In some cases, the members can recognize the same epitopes with substantially the same binding strength. It will be understood, however, that in some cases an affinity reagent can include two or more affinity moieties having different structures and different binding affinities compared to each other. Similarly, an affinity reagent can include two or more paratopes having different structures and different binding affinities compared to each other.

An affinity reagent of the present disclosure can include one or more labels. For example, an affinity reagent can include at least 1, 2, 3, 4, 5, 10, 15, 20 or more labels. Alternatively or additionally, an affinity reagent can include at most 20, 15, 10, 5, 4, 3, 2, or 1 labels. In some configurations, an affinity reagent can be attached to one or more labels. For example, an affinity reagent can include a particle (e.g., structured nucleic acid particle) that is attached to at least one paratope and further attached to at least one label. Methods and compositions set forth herein in the context of labels although exemplified for affinity reagents can be extended to other molecules such as analytes (e.g., proteins).

Multiple labels that are present in an affinity reagent can have the same structure as each other or they can differ structurally from each other. Optionally, multiple labels can have different detectable characteristics. For example, two or more optical labels can differ in terms of luminescence lifetime, luminescence polarity, extinction coefficient, quantum yield of luminescence, spectral region for absorbance, spectral region for excitation or spectral region for emission. Alternatively, two or more optical labels can have overlapping detectable characteristics, for example, in terms of luminescence lifetime, luminescence polarity, extinction coefficient, quantum yield of luminescence, spectral region for absorbance, spectral region for excitation or spectral region for emission. This can result from the two or more optical labels having the same structure, but in some cases two or more labels can have the same or overlapping detection properties despite having different structures.

A label of a molecule, such as an affinity reagent, can be exogenous or endogenous to the molecule. Exogenous labels can be attached to an affinity reagent or other molecule using methods and compositions known in the art such as artificial linkers or genetic fusions. A wide variety of labels can be used in a composition or method set forth herein including, for example, optically detectable labels, such as luminophores (e.g., fluorophores), enzymes (e.g., enzymes which catalyze reactions with colored reagents or products), electrochemical labels (e.g., highly charged moieties). Magnetic contrast imaging moieties can be used such as gadolinium-diethylenetriaminepentacetate (Gd-DTPA), gadolinium-dodecane tetraacetic acid (Gd-DOTA) or others used for magnetic resonance techniques. Moieties that are detected through subsequent processing, such as nucleic acid barcode labels, can also be useful. Nucleic acids can be detected or identified via nucleic acid amplification, sequencing or hybridization assays.

Any of a variety of luminophores may be used herein. Luminophores may include labels that emit in the ultraviolet, visible, or infrared region of the spectrum. In some cases, the luminophore may be selected from the group consisting of FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, R-Phycoerythrin, Allophcocyanin (APC). In some cases, the label may be an Atto dye, for example Atto 390, Atto 425, Atto 430, Atto 465, Atto 488, Atto 490, Atto 495, Atto 514, Atto 520, Atto 532, Atto 540, Atto 550, Atto 565, Atto 580, Atto 590, Atto 594, Atto 610, Atto 611, Atto 612, Atto 620, Atto 633, Atto 635, Atto 647, Atto 655, Atto 680, Atto 700, Atto 725, Atto 740, Atto MB2, Atto Oxa12, Atto Rho101, Atto Rho12, Atto Rho13, Atto Rho14, Atto Rho3B, Atto Rho6G, or Atto Thio12. In some cases, the luminophore may be a fluorescent protein such as green fluorescent protein (GFP), cyan fluorescent protein (CFP), red fluorescent protein (RFP), blue fluorescent protein (BFP), orange fluorescent protein (OFP), and yellow fluorescent protein (YFP). A wide range of effective luminophores are commercially available, for example, from the Molecular Probes division of ThermoFisher Scientific and/or generally described in the Molecular Probes Handbook (11th Edition) which is hereby incorporated by reference. Label components may also include intercalation dyes, such as ethidium bromide, propidium bromide, crystal violet, 4′,6-diamidino-2-phenylindole (DAPI), 7-aminoactinomycin D (7-AAD), Hoescht 33258, Hoescht 33342, Hoescht 34580, YOYO-1, DiYO-1, TOTO-1, DiTO-1, or combinations thereof.

Optionally, an affinity reagent can be attached to a solid support or particle. A particularly useful particle is a structured nucleic acid particle (e.g., nucleic acid origami), for example, having structural or functional characteristics set forth herein in the context of attachment to proteins and other analytes. An analyte, affinity reagent, docker, tether, label or other moiety can be attached to a nucleic acid origami via a scaffold component or oligonucleotide component. For example, the scaffold or oligonucleotide can include a nucleotide analog that forms a covalent or non-covalent bond with the attached moiety.

Any of a variety of chemistries can be used to attach an analyte, affinity reagent, docker, tether or other moiety to a solid support or particle (e.g., structured nucleic acid particle). The attachment can be covalent. Exemplary covalent chemistries include, but are not limited to, click chemistries or chemistries set forth in U.S. Pat. Nos. 11,203,612 or 11,505,796; or US Pat. App. Pub. No. 2022/0162684 A1, each of which is incorporated herein by reference. Another example is the SpyTag/SpyCatcher system (See, Zakeri et al. Proceedings Nat'l Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The SpyCatcher can function by irreversibly bonding to a SpyTag through an isopeptide bond. Any of a variety of non-covalent bonds can be used to attach an analyte, affinity reagent or other moiety to a solid support or particle (e.g., structured nucleic acid particle). Receptors and their ligands can be particularly useful. Examples include, but are not limited to, antibodies, antigens, (strept) avidin (or analogs thereof), biotin (or analogs thereof), affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach a functional moiety to a solid support or particle (e.g., structured nucleic acid particle). Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Reagents and techniques that can be used to non-covalently attach an affinity reagent or other moiety to a particle (e.g., structured nucleic acid particle) are set forth in U.S. Pat. Nos. 11,203,612 or 11,505,796; or US Pat. App. Pub. No. 2022/0162684 A1, each of which is incorporated herein by reference.

A structured nucleic acid particle that is made or used in a method set forth herein can be suspended in a fluid, immobilized on a solid support, or immobilized in another material such as a gel or solid support material. This can be the case before, during or after being attached to a moiety of interest, such as a tether, docker, linker, label, analyte or affinity moiety. For example, a population of structured nucleic acid particles can be colloidal for some, or all steps of a method set forth herein. Alternatively, a population of structured nucleic acid particles can be immobilized in, or on, a solid support for some, or all steps of a method set forth herein. For example, analytes or affinity reagents can be attached to addresses (or other unique identifiers) of an array via structured nucleic acid molecules.

A particle need not be composed primarily of nucleic acid and, in some cases, may be devoid of nucleic acids. For example, an analyte, affinity reagent, label, linker, docker or tether can be attached to an artificial polymer that is configured to form a particle. In other examples, a particle can be composed of a solid support material, such as those set forth herein, glass, silicon, silica, carbon, cellulose, polyethylene glycol (PEG), upconversion nanocrystal, or a quantum dot.

Delivery and removal of fluids will be set forth below in the context of fluid-phase affinity reagents and immobilized analytes (e.g., proteins attached to addresses of an array). This is done for ease of explanation. It will be understood that similar compositions and methods can be used for fluid-phase analytes and immobilized affinity reagents.

A method of the present disclosure can include a step of contacting an array of analytes with affinity reagents. Typically, the affinity reagents are in a fluid phase that is delivered to the array and the analytes are immobilized to addresses in the array. Any of a variety of fluidics techniques or apparatus can be employed for delivery of the fluid phase. For example, a fluid phase can be provided in a tube, pipette tip, syringe or the like and delivered via fluid displacement. In some cases, the array can be present in the lumen of a flow cell or other vessel having an inlet and outlet, and a fluid phase can enter the lumen via displacement of fluid through the inlet. The fluid phase can also exit the flow cell, at least in part, via displacement through the outlet. Other methods can be employed to deliver fluid to an array such as dipping the array into a vessel containing the fluid, flowing fluid through a nozzle to the array, or spin coating fluid on the surface of the array.

A fluid phase that is in contact with an array can contain affinity reagents in a desired quantity as measured, for example, by concentration, mass or number of affinity reagent molecules. For example, the concentration of affinity reagents in contact with an array can be at least 10 nM, 100 nM, 1 μM, 10 μM, 100 μM, 1 mM, 10 mM, 100 mM, or higher. Alternatively or additionally, the concentration of affinity reagents in contact with an array can be at most 100 mM, 10 mM, 1 mM, 100 μM, 10 M, 1 μM, 100 nM, 10 nM, or lower. The quantity of affinity reagents in a fluid phase is typically greater than the quantity of analytes in an array to which the fluid is in contact. For example, a fluid phase can contain a number of affinity reagent molecules that is at least 1.5×, 2×, 5×, 10×, 50×, 100×, 1000×, 10000× or greater relative to the number of analytes in an array to which the fluid phase is in contact. However, in some cases the fluid can contain an equivalent or lower quantity of affinity reagents compared to the quantity of analytes in the array. For example, a fluid phase can contain a number of affinity reagent molecules that is at most 1×, 0.5×, 0.1×, 0.01× or less relative to the number of analytes in an array to which the fluid phase is in contact.

In particular configurations of the methods set forth herein, a fluid phase is contacted with an array and the affinity reagents are present at a first concentration in the fluid phase. A plurality of the affinity reagents can then be removed, thereby retaining a fraction of the affinity reagents in contact with the array. Thus, the quantity of affinity reagents in contact with the array is reduced. The fraction of affinity reagents can be contained in a fluid and can be present at a second concentration that is higher, equal or lower compared to the first concentration. For example, the concentration of affinity reagents can be reduced for the retained fraction if the fluid volume is equivalent before and after removal of the plurality of affinity reagents. Alternatively, a higher concentration of affinity reagents can result for the retained fraction if the volume of fluid for the retained fraction is substantially lower after removal of the plurality of affinity reagents.

Removal of fluid from an array can be carried out using any of a variety of fluidics techniques or apparatus including, but not limited to, those set forth above in the context of delivering fluids to an array. For example, at least a portion of the fluid in contact with an array can be removed by fluid displacement. The fluid can be displaced with an equivalent volume of liquid or gas. Displacement with gas can be particularly useful for reducing the volume of fluid in contact with the array. Liquid displacement can be performed to result in replacement of at least a portion of a first fluid phase with a second fluid phase. Displacement with a second fluid phase can be useful for adding or replacing reagents, cofactors, salts, or other fluid components. For example, the second fluid can contain affinity reagents of the same or different type as the first fluid that is displaced. Other methods that can be employed to remove affinity reagents from an array or vessel include, for example, precipitation of the affinity reagents, solid-phase extraction of the affinity reagents, liquid-liquid extraction of the affinity reagents, capture of the affinity reagents via a solid-phase receptor or ligand that binds to the affinity reagents, or dialysis of the affinity reagents.

Detection techniques and apparatus will be set forth below in the context of fluid-phase affinity reagents and immobilized analytes (e.g., proteins attached to addresses of an array). This is done for ease of explanation. It will be understood that similar compositions and methods can be used for fluid-phase analytes and immobilized affinity reagents.

A method of the present disclosure can include a step of detecting binding of affinity reagents to analytes in an array. Binding of affinity reagents with analytes can be detected using any of a variety of techniques that are appropriate to the assay components used. For example, an affinity reagent can be detected at a unique identifier (e.g., address) in an array by acquiring a signal from a label attached to the affinity reagent when bound to an analyte at the unique identifier. In some configurations, a complex between an affinity reagent and analyte need not be directly detected, for example, in formats where a nucleic acid tag or other moiety is created or modified as a result of binding. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary detection techniques and apparatus are set forth in U.S. Pat. No. 10,473,654 or US Pat. App. Pub. No. 2022/0162684 A1, each of which is incorporated herein by reference. A detection technique used in a method set forth herein can be configured to resolve addresses (or other unique identifiers) of an array. For example, a detection technique can be configured for single-molecule resolution of analytes.

In some configurations of the methods set forth herein, affinity reagents that are not bound to analytes at addresses of an array are removed from contact with the array prior to detecting affinity reagents that are bound to the addresses. Removal of non-bound affinity reagents can provide the advantage of reducing unwanted background when detecting bound affinity reagents. This can be particularly helpful when affinity reagents have labels that produce detectable signals in both the bound and non-bound state. One option to avoid or reduce unwanted background signals is to remove non-bound affinity reagents. However, this approach may change the dynamics of binding between affinity reagent and analyte unless remediation is performed, such as detecting bound affinity reagents on a time scale that is faster than the rate at which binding equilibrium is substantially shifted. Another option for reducing unwanted signal from non-bound affinity reagents is to use a detection apparatus that is capable of spatially confined collection of signals. For example, total internal reflectance can be used to collect signals for affinity reagents bound at or near an array surface while rejecting signals produced further away from the surface of the array. Another example is the use of waveguides such as zero mode waveguides for spatially resolved excitation of labels and/or spatially resolved acquisition of emission signals from excited labels. Other examples of spatially resolved detection of labeled affinity reagents include, but are not limited to, detection of luminescent moieties using confocal fluorescence microscopy, detection of affinity reagents using surface plasmon resonance (SPR) or detection of charged moieties using field effect transistors.

Any of a variety of proximity-based detection techniques can be used to detect binding of affinity reagents to analytes. A particularly useful method is detection of energy transfer between a donor and acceptor. For example, Förster resonance energy transfer (FRET) can be detected between a first luminophore (i.e., donor) and second luminophore (i.e. acceptor). Energy transfer techniques utilize a donor that is excited by an energy source, such as a laser or other radiation source, and an acceptor that receives energy from the donor to, in turn, produce a detectable signal, such as a luminescence signal. The efficiency of energy transfer is inversely proportional to distance between donor and acceptor, making energy transfer sensitive to proximity on a scale that is comparable to the distances between affinity reagents and the analytes to which they bind.

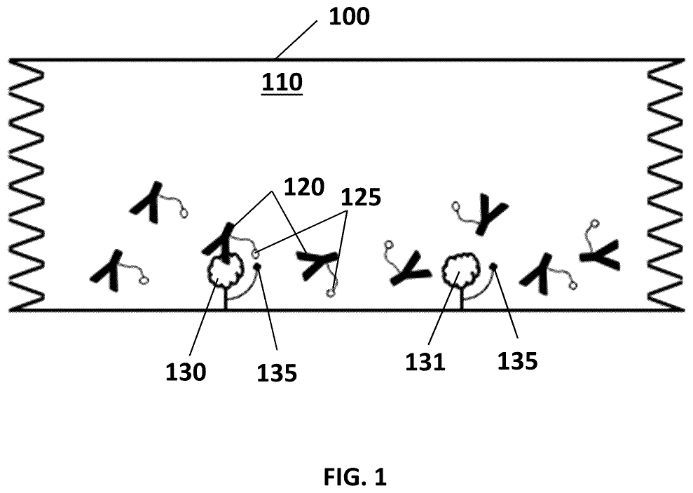

FIG. 1 shows a cross section of a region of a flow cell 100 having two protein analytes (130, 131) immobilized on the lower surface and a fluid phase 110 containing affinity reagents 120, wherein the affinity reagents 120 are attached to a first luminophore 125 and the protein analytes are attached to a second luminophore 135. The first luminophore 125 and second luminophore 135 are capable of FRET when in proximity to each other, with either being the donor and the other being the acceptor. Using appropriate optics including, for example, a filter to selectively collect emission from the acceptor and to reject emission from the donor, binding between the affinity reagent and protein can be detected in the presence of unbound affinity reagent. Thus, detection can occur while equilibrium is maintained due to the concentration of free affinity reagent in solution phase; however, emission produced by free affinity reagent does not produce substantial background interference. Returning to FIG. 1, FRET can be detected due to binding of the affinity reagent 120 to the protein 130 and absence of signal from the address for the protein 131 is indicative of the affinity reagent 120 not recognizing nor binding to the protein 131. It will be understood that FRET can be used similarly if the protein is in fluid phase and the affinity reagent is immobilized on the lower surface of the flow cell. The use of FRET in the context of FIG. 1 is exemplary and it will be understood that other proximity-based detection techniques can be used instead.

Other proximity-based detection techniques that can be used in a method set forth herein include those that employ various pairs of components that produce unique signals when in proximity to each other compared to when they are apart. For example, a luminophore component and quencher component can be used to detect proximity via quenching of signal that would otherwise be produced by the luminophore. A two component luminophore systems can also be useful. For example, fluorogen-activated-proteins utilize a protein component and fluorogen component to produce unique fluorescent signal when in proximity to each other (see, for example, Gallo, Bioconjugate Chem. 31:16-27 (2020). In another example, an intercalating dye component and nucleic acid component can produce a unique optical signal when in proximity to each other. Also useful are detection components used in split protein assays such as two parts of an enzyme that are uniquely functional when in proximity to each other. Exemplary enzymes that can be split into components for use in proximity-based detection include, but are not limited to beta lactamase, dihydrofolate reductase, focal adhesion kinase, Green Fluorescent Protein (and variants thereof), horseradish peroxidase, and luciferase.

An analyte or a unique identifier (e.g., address) to which an analyte is attached can be co-localized with a first component of a proximity-based detection pair (e.g., a FRET donor), for example, via covalent or non-covalent attachment; and an affinity reagent can be co-localized with a second component of the proximity-based detection pair (e.g., a FRET acceptor), for example, via covalent or non-covalent attachment. The opposite placement of donor and acceptor is also possible (i.e., the analyte or its unique identifier can be co-localized with the acceptor, and the affinity reagent can be co-localized with the donor). Using a pair of proximity-based detection components (e.g., donor and acceptor) allows interaction between affinity reagent and analyte to be distinguished even in the presence of unbound affinity reagent.

Proximity-based detection can be facilitated by dockers and tethers. An analyte used in a composition or method of the present disclosure can have a docker and the docker can be attached to a first component of a proximity-based detection pair (e.g., a FRET donor or acceptor used for energy transfer). An affinity reagent used in a composition or method of the present disclosure can have a tether and the tether can be attached to a second component of a proximity-based detection pair (e.g., a FRET donor or acceptor used for energy transfer). The docker and tether can be configured to position the components of the pair in proximity to each other when the affinity reagent is bound to the analyte. As such, the docker and tether can facilitate proximity-based detection (e.g., energy transfer) and, thus, facilitate detection of binding between the affinity reagent and analyte.

Some compositions, systems, and methods provided in the present disclosure can utilize FRET detection as a method for detecting presence or absence of a binding interaction between an analyte and an affinity reagent. It may be advantageous to configure a system or composition to generate a FRET-based signal by positioning an acceptor fluorescent molecule within a close proximity to two or more donor fluorescent molecules. In compositions, systems, and method provided herein, binding of an affinity reagent to an analyte can bring a first reactant co-localized with the affinity reagent in sufficient proximity to a second reactant co-localized with the analyte to form a binding interaction between the first reactant and the second reactant, thereby bringing an acceptor fluorescent molecule into proximity of one or more donor fluorescent molecules. The skilled person will readily recognize that FRET-based detection system provided herein can readily be reversed with respect to which entity (e.g., analyte, affinity reagent) is co-localized with an acceptor fluorescent molecule or donor fluorescent molecule(s).

In an aspect, provided herein is a composition, comprising: (a) an analyte, wherein the analyte is co-localized with a first reactant, and wherein the first reactant is attached to a plurality of donor fluorescent molecules, and (b) an affinity reagent, wherein the affinity reagent is attached to a second reactant, and wherein the affinity reagent is further attached to an acceptor fluorescent molecule, wherein the affinity reagent is configured to form a first binding interaction with the analyte, and wherein the first reactant is configured to form a second binding interaction with the second reactant that brings the acceptor fluorescent molecule within a distance from the plurality of donor fluorescent molecules to produce an optical signal in the presence of a photon.

In another aspect, provided herein is a method, comprising: (a) binding an affinity reagent to an analyte, wherein the analyte is co-localized with a first reactant, wherein the first reactant is attached to a plurality of donor fluorescent molecules, wherein the affinity reagent is co-localized with a second reactant, and wherein the affinity reagent is further attached to an acceptor fluorescent molecule, (b) forming a binding interaction between the first reactant and the second reactant, thereby bringing the acceptor fluorescent molecule within a distance from the plurality of donor fluorescent molecules, and (c) after forming the binding interaction between the first reactant and the second reactant, detecting an optical signal from the acceptor fluorescent molecule.

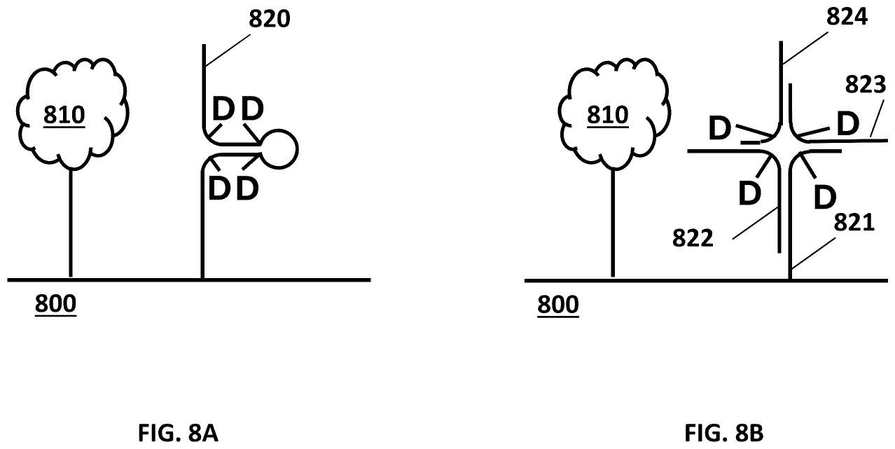

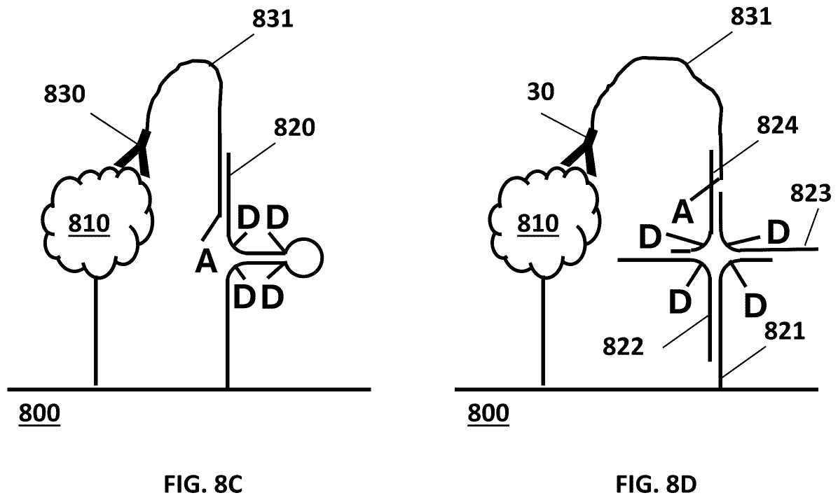

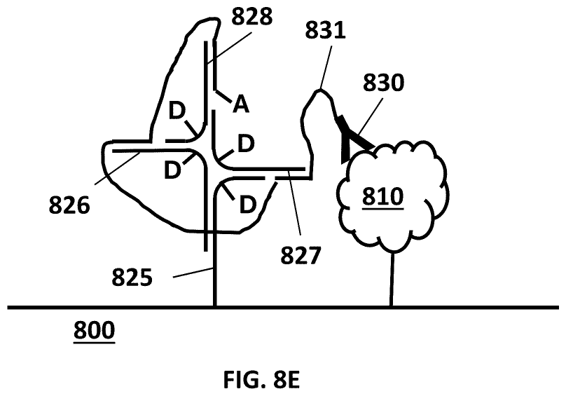

FIGS. 8A-8E illustrate configurations of FRET-based detection systems comprising pluralities of donor fluorescent molecules. FIG. 8A depicts an analyte 810 co-localized at an address 800 (e.g., of a solid support; on a particle) with a labeled moiety 820. A plurality of donor fluorescent molecules (D) is attached to the labeled moiety 820. In some configurations, the labeled moiety 820 is an oligonucleotide. The oligonucleotide may comprise regions of self-complementarity, thereby bringing the donor fluorescent molecules into a close proximity with each other. FIG. 8B depicts an alternative configuration, in which an oligonucleotide complex is formed by hybridizing a plurality of oligonucleotides (821, 822, 823, 824), at least one of which is attached at address 800. Donor fluorescent molecules (D) may be attached to two or more oligonucleotides of the plurality of oligonucleotides of the oligonucleotide complex. The oligonucleotide-based configurations of FIGS. 8A and 8B may be advantageous as dye attachment positions can be chosen to predictably locate the donor fluorescent molecules in sufficiently close proximity to each other.

FIGS. 8C and 8D depict configurations of the compositions depicted in FIGS. 8A and 8B, respectively, when an affinity reagent 830 is coupled to the analyte. FIG. 8C depicts an affinity reagent 830 coupled to the analyte 810. The affinity reagent 830 is attached to a linking moiety 831 that is attached to an acceptor fluorescent molecule (A). For oligonucleotide-based configurations, the linking moiety 831 may comprise an oligonucleotide that hybridizes to an oligonucleotide co-localized with the analyte 810. The acceptor fluorescent molecule (A) is attached to the linking moiety 831 in a position that brings the acceptor fluorescent molecule in proximity (e.g., an average distance of less than about 10 nm, 9 nm, 8 nm, 7 nm, 6 nm, 5 nm, 4 nm, 3 nm, 2 nm, or less than 1 nm) to the plurality of donor fluorescent molecules (D). FIG. 8D depicts binding of the linking moiety 831 attached to the affinity reagent 830 to the oligonucleotide complex. The oligonucleotide complex may comprise a plurality of single-stranded nucleic acids (e.g., portions of the oligonucleotides that are not hybridized to other oligonucleotides of the oligonucleotide complex). At least one of the single-stranded nucleic acids may comprise a moiety that can be bound by the linking moiety 831. Preferably, a plurality of single-stranded nucleic acids of the oligonucleotide complex can be bound by the linking moiety 831. Such a configuration may be advantageous because dissociation of the linking moiety 831 from a single-stranded nucleic acid of the oligonucleotide complex can be offset by association of the linking moiety 831 to a different single-stranded nucleic acid. FIG. 8E further extends the oligonucleotide complex configuration of FIGS. 8B and 8D to include a kinetic trap, as set forth herein. The linking moiety 831 can comprise a plurality of moieties that couple to moieties of the labeled moiety, such as the single-stranded nucleic acids of the oligonucleotide complex. An oligonucleotide complex may be useful because the relative positioning of coupling moieties can be utilized to control the rate of formation of the kinetic trap. An acceptor fluorescent molecule can be attached to a linking moiety 831 such that it is brought into proximity to a plurality of donor fluorescent molecules when a final interaction of a kinetic trap has been formed.

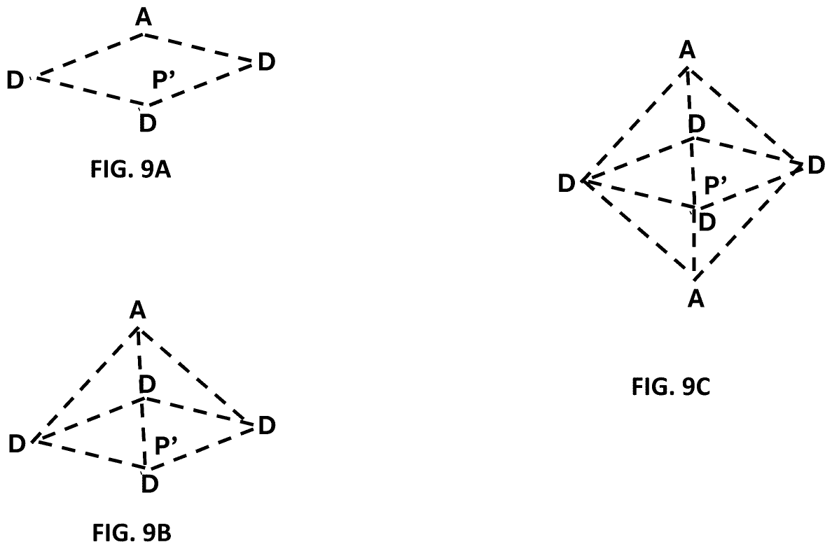

FIGS. 9A-9C illustrate example of the relative spatial configurations of acceptor and donor fluorescent molecules in a FRET-based detection system. In the configuration of FIG. 9A, an acceptor fluorescent molecule (A) can be located in a substantially coplanar arrangement with 3 or more donor fluorescent molecules (D). Preferably, the acceptor fluorescent molecule is located no more than about 10 nm (e.g., less than 10 nm, 9 nm, 8 nm, 7 nm, 6 nm, 5 nm, 4 nm, 3 nm, 2 nm, or less than 1 nm) from the furthest donor fluorescent molecule D. In the configuration of FIG. 9B, four donor fluorescent molecules (D) are positioned in a substantially coplanar configuration, and an acceptor fluorescent molecule (A) is located at a position that is substantially equidistant to each of the donor fluorescent molecules (e.g., an average distance of less than 10 nm, 9 nm, 8 nm, 7 nm, 6 nm, 5 nm, 4 nm, 3 nm, 2 nm, or less than 1 nm from the plurality of donor fluorescent molecules). The configuration of FIG. 9C is similar to that of FIG. 9B, but includes an additional acceptor fluorescent molecule that can receive energy transfer from the donor molecules. The skilled person will readily recognize that attachment of fluorescent molecules to scaffolds such as nucleic acids can facilitate some control over the spatial position and orientation of the fluorescent molecules, although the highly symmetrical alignments of FIGS. 9A-9C may not be exactly achievable. Further, natural molecular motions will produce some degree of spatial and temporal variability in the relative positions of fluorescent molecules in the systems set forth herein. The skilled person can readily envisage numerous configurations that facilitate positioning of an acceptor fluorescent molecule in close proximity to a plurality of donor fluorescent molecules to increase the efficiency of energy transfer in the FRET-based system.

Although the FRET-based systems depicted in FIGS. 8A-8E are exemplified with oligonucleotides to form proximity-based binding interactions that facilitate formation of a FRET signal, various other binding compositions set forth herein may be utilized to form the proximity-based binding interaction, such as covalent reactants, receptor-ligand binding pairs, and chelating compounds (e.g., IMAC-based compounds) with associated ligands.

An affinity reagent may be configured to be detected by a FRET-based signal. An affinity reagent may be coupled to one or more dye molecules that can facilitate providing a FRET signal (e.g., one or more acceptor fluorescent molecules, one or more donor fluorescent molecules). An affinity reagent may be coupled or attached to one or more fluorescent molecules by a linking moiety, as set forth herein. In some cases, an affinity reagent may be attached or coupled to a first fluorescent molecule and a second fluorescent molecule, wherein the affinity reagent is attached to a linking moiety, and wherein the linking moiety is attached to the first fluorescent molecule and the second fluorescent molecule. In other cases, an affinity reagent may be attached or coupled to a first fluorescent molecule and a second fluorescent molecule, wherein the affinity reagent is attached to a first linking moiety and a second linking moiety, wherein the first linking moiety is attached to the first fluorescent molecule, and wherein the second linking moiety is attached to the second fluorescent molecule.

An affinity reagent that is detected by a FRET-based signal may be configured to form a binding interaction that facilitates providing the FRET-based signal when the affinity reagent is bound to an analyte. Further, a FRET-based signal may not be detected when the affinity reagent is not bound to the analyte (e.g., the affinity reagent is in a fluid phase). Accordingly, a composition may comprise a fluidic medium contacted to a solid support and/or an analyte. In some cases, the fluidic medium may be further contacted to an affinity reagent that is coupled to the analyte. In this configuration, a signal may be detected from the affinity reagent bound to the analyte. In other cases, the fluidic medium may comprise an affinity reagent that is in the fluid phase (e.g., the affinity reagent is not coupled to an analyte). In this configuration, no signal may be detected from the affinity reagent bound to the analyte.

Fluorescence-based detection methods (e.g., FRET-based detection) may utilize light fields to facilitate the providing of a fluorescent signal. Accordingly, a composition set forth herein may comprise a photon. A photon in a system set forth herein may have a wavelength that is absorbed by a fluorescent molecule (e.g., a donor fluorescent molecule). Alternatively, a photon in a system set forth herein may have a wavelength that is emitted by a fluorescent molecule. Excitation and emission wavelengths for fluorescent molecules are well known to a skilled person. A system, as set forth herein, may comprise a device that is configured to provide a photon of light to a fluorescence-based composition. Exemplary light-producing devices can include a laser, a light bulb, a light-emitting diode, a filament, or a lamp.

Systems, compositions, and methods utilizing dockers and tethers are provided in U.S. Patent Publication No. 20240426839, which is herein incorporated by reference. A particularly useful docker/tether system comprises a nucleic acid docker strand that is complementary to a nucleic acid tether strand. In some cases, an analyte can be co-localized with a single docker or, alternatively, with a plurality of dockers. Likewise, an affinity reagent can be co-localized with a single tether or a plurality of tethers. Optionally, an analyte, affinity reagent, docker, tether, label or other moiety can be attached to a solid support or particle. A particularly useful particle is a structured nucleic acid particle (e.g., nucleic acid origami), for example, having structural or functional characteristics set forth herein in the context of attachment to proteins and other analytes. An analyte, affinity reagent, docker, tether, label or other moiety can be attached to a nucleic acid origami via a scaffold component or oligonucleotide component. For example, the scaffold or oligonucleotide can include a nucleotide analog that forms a covalent or non-covalent bond.

FIGS. 6A-6G depict alternative tether-docker schemes that utilize a single tether strand and multiple docker strands. The binding of the single tether strand to the multiple docker strands may be configured to occur as a series or sequence of binding interactions that collectively bind an affinity reagent attached to the docker to an array address. Preferably, the single tether strand and the multiple docker strands are configured as a set of weak interactions that are unlikely to stabilize unless the attached affinity reagent is bound to an analyte for a sufficient time to facilitate the series or sequence of weak interactions. In some cases, each docker-tether interaction of the multiple dockers has an orthogonal binding specificity to any other docker-tether interaction. In other cases, two or more docker-tether interactions of the multiple dockers have an identical binding specificity.

FIG. 6A depicts an address of a solid support 600 that is attached to an analyte 610 and three unique docker oligonucleotides (605A, 605B, and 605C, respectively). The analyte is bound by an affinity reagent 620 that is attached to a tether strand that comprises complementary nucleotide sequences (615A, 615B, and 615C) that are joined by linking moieties. FIG. 6B depicts a second configuration of the system, in which the tether strand has become partially bound by the hybridization of complementary nucleotide sequence 615A with docker oligonucleotide 605A. The hybridization brings the remaining unbound complementary nucleotide sequences closer to the docker oligonucleotides to which they are complementary. FIG. 6C depicts a third configuration, in which the tether strand has become further bound by the hybridization of complementary nucleotide sequence 615B with docker oligonucleotide 605B. FIG. 6D depicts a fourth configuration, in which the tether strand has become further bound by the hybridization of complementary nucleotide sequence 615C with docker oligonucleotide 605C. As the number of binding interactions between the affinity reagent 620 and the tether strand with the analyte 610 and docker strands increases, the stability of the binding of the affinity reagent 620 at the address of the solid support 600 may increase.

FIGS. 6E-6G depict an alternative configuration that utilizes secondary structures of the tether strand to control the rate of binding to the docker strands. FIG. 6E depicts a similar configuration to the configuration shown in FIG. 6A. The tether strand comprises two complementary nucleotide sequences (615A and 615B), each of which forms a stem-loop structure by hybridization with internally complementary nucleotide sequences (625A and 625B, respectively). The address of the solid support 600 is attached to two docker oligonucleotides 605A and 605B. FIG. 6F depicts a second configuration, in which docker oligonucleotide 605A has displaced internally complementary nucleotide sequence 625A, thereby facilitating binding of docker oligonucleotide 605A to complementary nucleotide sequence 615A. FIG. 6G depicts a third configuration, in which docker oligonucleotide 605B has displaced internally complementary nucleotide sequence 625B, thereby facilitating binding of docker oligonucleotide 605B to complementary nucleotide sequence 615B.

The single tether strand systems depicted in FIGS. 6A-6G need not be limited to nucleic acid-based interactions. Other moieties that form covalent or non-covalent interactions may be utilized. For example, a final interaction in a sequence of tether-docker interactions may be a non-dissociable interaction, such as a click-type reaction or a streptavidin-biotin interaction. Such an interaction may facilitate retention of an affinity reagent at an array address, but may only occur if the affinity reagent is bound to an analyte for a sufficient time to further form the full sequence of tether-docker interactions.

In an aspect, provided herein is a method, comprising: (a) providing an analyte, wherein the analyte is associated to a first docker strand and a second docker strand, (b) binding an affinity reagent to the analyte, wherein the affinity reagent is attached to a tether strand, and (c) after binding the affinity reagent to the analyte, binding the tether strand of the affinity reagent to the first docker strand and the second docker strand. In another aspect, provided herein is a composition, comprising: (a) an analyte, wherein the analyte is co-localized with a first docker strand and a second docker strand, and (b) an affinity reagent coupled to the analyte, wherein the affinity reagent is attached to a tether strand, wherein the tether strand is coupled to the first docker strand and the second docker strand. In another aspect, provided herein is a system, comprising: (a) a plurality of analytes, wherein each analyte is individually co-localized with a first docker strand, a second docker strand, and a unique identifier, (b) a plurality of affinity reagents, wherein each affinity reagent of the plurality of affinity reagents is individually attached to a tether strand, wherein the tether strand comprises a first moiety that is complementary to the first docker strand, wherein the tether strand further comprises a second moiety that is complementary to the second docker strand, and wherein each affinity reagent of the plurality of affinity reagents further comprises a detectable label, and (c) a detection device, wherein the detection device is configured to detect signals from detectable labels at unique identifiers associated with analytes of the plurality of analytes. In some cases, a method may comprise simultaneously binding a tether strand of an affinity reagent to a first docker strand and a second docker strand. In other cases, a method may comprise sequentially binding the tether strand of the affinity reagent to the first docker strand and the second docker strand.

A single tether system, such as the systems depicted in FIGS. 6A-6G, may comprise a kinetic trap. The initial interaction of an affinity reagent with an analyte can bring a first tether-docker pair into sufficient proximity to form a tether-docker interaction. In turn, the reduced range of motion of the tether strand increases the likelihood of a second tether-docker pair forming a tether-docker interaction. Accordingly, each successive tether-docker interaction formation increases the rate of the next interaction forming until the tether strand is fully bound to most or all available dockers. The skilled person will readily recognize numerous configurations that facilitate tuning of the kinetics of the tether-docker interactions to adjust the avidity of an affinity reagent attached to the tether. For example, an affinity reagent with a faster off-rate may be provided with a tether strand that is configured to more rapidly form the kinetic trap, thereby increasing the likelihood of the affinity reagent being detected while bound to an analyte. In another example, an affinity reagent with a slower on-rate may be provided with a tether strand that is configured to more slowly form the kinetic trap, thereby reducing a likelihood of a false negative detection event.

A system that is configured to form a kinetic trap that retains an affinity reagent at an address of a solid support can comprise an affinity reagent attached to only one tether strand, and a plurality of docker strands at the address of the solid support. The only one tether strand may comprise a plurality of moieties that are configured to couple to the plurality of docker strands via individual binding interactions. The individual binding interactions may be configured to occur in a sequence. In some cases, each successive formed binding interaction in a sequence of binding interactions may be increasingly stable. For example, a nucleic acid tether strand may comprise a set of nucleotide sequences, each of which is complementary to a different docker oligonucleotide. The first binding interaction in a sequence of binding interactions between the set of oligonucleotide sequences and the docker oligonucleotides may be configured to have 6 nucleotides of hybridization, thereby forming a weak binding interaction. The second binding interaction in the sequence of binding interactions between the set of oligonucleotide sequences and the docker oligonucleotides may be configured to have 10 nucleotides of hybridization, thereby forming a stronger binding interaction than the first binding interaction. Subsequent binding interactions may have increasingly larger sequence complementarity, thereby increasing the stability of the overall complex with each successive binding interaction. Alternatively, an affinity reagent may be attached to two or more tether strands, with at least one tether strand configured to form a kinetic trap, as set forth herein.

The skilled person will recognize numerous variations of tether-docker systems that can be utilized to control the kinetics of a kinetic trap such as the one shown in FIGS. 6A-6G. The only one tether strand attached to an affinity reagent may comprise a plurality of moieties that are configured to bind to a plurality of dockers. A tether strand may comprise at least about N moieties that are configured to bind to a plurality of dockers, where N can be at least about 2, 3, 4, 5, 6, 7, 8, 9, 10, 20, or more than 20 moieties. Alternatively or additionally, N can be no more than about 20, 10, 9, 8, 7, 6, 5, 4, 3, or less than 3 moieties. An address of a solid support may be attached to at least M dockers, where M can be less than or equal to the N moieties of a tether strand. For example, each of the N moieties of a tether strand may be configured to bind to a unique docker of a set of N dockers. Alternatively, a docker strand may be configured to bind to two or more of the N moieties of a tether strand, therefore requiring fewer dockers than there are moieties of a tether strand.

A tether strand may comprise an oligonucleotide that comprises a plurality of moieties that are configured to bind to differing docker strands, in which each of the plurality of moieties comprises a residue sequence. In some cases, binding a tether strand of an affinity reagent to a first docker strand and a second docker strand can comprise binding a first residue sequence of the tether strand to a residue sequence of the first docker strand and binding a second residue sequence of the tether strand to a residue sequence of the second docker strand. The rate of forming a binding interaction between a tether strand and a docker strand may be further controlled by providing a region of internal complementarity to a nucleotide sequence of the tether strand or docker strand. Accordingly, a method may further comprise disrupting the region of internal complementarity of a residue sequence (e.g., performing a toehold displacement of a region of internal complementarity of a tether strand by a residue sequence of the first docker strand).

GC content of a docker strand and/or a tether strand may be utilized to control the stability and kinetics of forming the binding interaction between the docker strand and tether strand. In a kinetic trap configuration, it may be preferable to sequence interactions to become increasingly stable. Accordingly, a first docker strand and its complementary sequence in a tether strand may have a lower GC content than a second docker strand and its complementary sequence in the tether strand. Further, length of sequence complementarity between a docker strand and a tether strand may be utilized to control the stability and kinetics of forming the binding interaction between the docker strand and tether strand. For example, a four nucleotide sequence may be more likely to dissociate from its complement than an eight nucleotide sequence. Accordingly, a first docker strand and its complementary sequence in a tether strand may have a shorter sequence length than a second docker strand and its complementary sequence in the tether strand. A docker strand may have a sequence complementarity length with a sequence of a tether strand of at least about 3, 4, 5, 6, 7, 8, 9, 10, 11, 12, 13, 14, 15, 16, 17, 18, 19, 20, 25, 30, 40, 50, or more than 50 nucleotides. Alternatively or additionally, a docker strand may have a sequence complementarity length with a sequence of a tether strand of no more than about 50, 40, 30, 25, 20, 19, 18, 17, 16, 15, 14, 13, 12, 11, 10, 9, 8, 7, 6, 5, 4, 3, or less than 3 nucleotides.

An affinity reagent that is attached to only one tether strand may form a sequence of binding interactions that fix the affinity reagent at an address. The first binding interaction of the sequence of binding interactions may be the binding of the affinity reagent to an analyte. The subsequent binding interactions of the sequence of binding interactions may be a plurality of binding interactions between the only one tether strand and a plurality of docker strands. A plurality of dockers that are configured to bind to a single tether strand may be spatially arranged to facilitate a sequencing of binding interactions between the plurality of dockers and the single tether strand. For example, the first docker strand that is configured to be bound by a single tether strand may be placed a distance X from an analyte, then a second docker strand may be located a distance Y from the first docker strand, where X>Y. Accordingly, the volume of space surrounding the first docker strand which a tether strand must explore to interact with the first docker strand may be larger than the volume of space surrounding the second docker strand which the tether strand must subsequently explore to then interact with the second docker strand. It may be advantageous to attach an analyte and an associated plurality of docker strands to a particle (e.g., a nucleic acid nanoparticle) that provides tunable spatial arrangement of the docker strands relative to the analyte.

A first docker strand may be located by a separation distance from a second docker strand, for example as measured by distance between points of attachment to a solid support or particle. The separation distance between the first docker strand and the second docker strand may be less than, equal to, or greater than the separation distance between the first docker strand and an analyte. A first docker strand may be located by a separation distance of at least about 1 nm, 2 nm, 3 nm, 4 nm, 5 nm, 10 nm, 15 nm, 20 nm, 30 nm, or more than 30 nm from a second docker strand. Alternatively or additionally, a first docker strand may be located by a separation distance of no more than about 30 nm, 20 nm, 15 nm, 10 nm, 5 nm, 4 nm, 3 nm, 2 nm, 1 nm, or less than 1 nm from a second docker strand. The docker strands of a set of at least 3 docker strands may be spaced by equal separation distances or differing separation distances depending upon the configuration.

A tether strand may comprise a plurality of moieties that are configured to individually form binding interactions with a plurality of docker strands. The moieties of a tether strand (e.g., nucleotide sequences) may be spaced along the tether strand at distances that facilitate control of the rate of forming binding interactions with docker strands. For example, if a first moiety of a docker strand has bound to a first docker strand, the rate of a second interaction forming between a second moiety of the tether strand and a second docker strand may be increased by separating the first moiety from the second moiety by a distance that is substantially equal or slight exceeding the separation distance between the first docker strand and the second docker strand. If the separation distance between the first moiety and the second moiety is further increased, the rate of forming the second interaction may decrease. A tether strand may comprise a plurality of moieties that interact with docker strands, in which each moiety of the plurality of moieties is joined to an adjacent moiety by a linking moiety, as set forth herein. The length of a linking moiety of a tether strand may be chosen to provide a particular separation distance between adjacent docker-interacting moieties of the tether strand. Linking moieties may be provided to a tether strand with differing or substantially equal lengths.

A first docker-interacting moiety of a tether strand may be located by a separation distance of at least about 1 nm, 2 nm, 3 nm, 4 nm, 5 nm, 10 nm, 15 nm, 20 nm, 30 nm, 40 nm, 50 nm, or more than 50 nm from a second docker-interacting moiety of the tether strand. Alternatively or additionally, a first docker-interacting moiety of a tether strand may be located by a separation distance of no more than about 30 nm, 20 nm, 15 nm, 10 nm, 5 nm, 4 nm, 3 nm, 2 nm, 1 nm, or less than 1 nm from a second docker-interacting moiety of a tether strand.

The skilled person will readily recognize that a tether strand must have a minimum length to form an interaction with a docker strand based upon the location of the docker strand relative to its associated analyte. For example, in FIG. 6B, the tether strand may require a linking moiety that is sufficiently long to extend from the affinity reagent 620 where it is bound to the analyte 610 to the first docker strand 605A. Likewise, forming the configuration of FIG. 6C may require a second linking moiety that is long enough to extend from the first docker strand 605A to the second docker strand 605B. The relative size and spacing of components such as dockers, tethers, and analytes may facilitate control of the rate of formation of a kinetic trap. For example, the length of a linking moiety between an affinity reagent and a first moiety of a tether strand that interacts with a docker strand may determine in part how long the tether strand may diffuse before contacting the docker strand (i.e., a longer linking moiety will require a longer time on average to diffuse a complementary region of the tether strand into proximity of a docker strand). In another example, a separation distance between a docker strand and an associated analyte may determine in part how long the tether strand may diffuse before contacting the docker strand (i.e., a larger separation distance will require a longer time on average to diffuse a complementary region of the tether strand into proximity of a docker strand). Lengths of linking moieties of a tether strand and separation distance of docker strands and/or analytes may be utilized to tune the rate of formation of a kinetic trap.

In some configurations, a binding reagent may comprise an affinity reagent, a first moiety, and a second moiety, wherein the first moiety is complementary to a first docker strand and the second moiety is complementary to a second docker strand, and wherein the affinity reagent is attached to the first moiety by a first linking moiety and the second moiety is attached to the first moiety by a second linking moiety. In such configurations, a length of the first linking moiety may be greater than a separation distance between the analyte and the first docker strand, and/or a length of the second linking moiety may be greater than a separation distance between the first docker strand and the second docker strand. A ratio of a length of a linking moiety (e.g., in a linear or extended state) to a separation distance (e.g., docker strand to analyte, docker strand to docker strand) may be at least about 1, 1.1, 1.2, 1.5, 2, 3, 4, 5, 10, 20, 50, 100, or more than 100. Alternatively or additionally, a ratio of a length of a linking moiety to a separation distance may be no more than about 100, 50, 20, 10, 5, 4, 3, 2, 1.5, 1.2, 1.1, or less than 1.1.

In some configurations, an affinity reagent may be attached to only one tether strand, wherein the tether strand comprises a detectable label that is configured to form a FRET pair. For a tether strand that interacts with a plurality of docker strands, the system may be configured to provide a FRET signal if the final binding interaction of a sequence of binding interactions has been formed. For example, the system of FIGS. 6A-6D may be modified to include a first FRET-based dye on docker strand 605C and a second FRET-based dye on moiety 615C. Accordingly, a FRET signal may only be observed if the configuration of FIG. 6D is formed. In some configurations, FRET-based dyes may be provided to two or more docker-interacting moieties of a plurality of moieties of a tether strand, and complementary FRET-based dyes may be provided to corresponding docker strands. Accordingly, the magnitude of a FRET signal may indicate the extent of binding interaction formation of a kinetic trap containing a single tether strand and a plurality of docker strands.

Any of a variety of chemistries can be used to attach an analyte, affinity reagent, docker, tether or other moiety to a solid support or particle (e.g., structured nucleic acid particle). The attachment can be covalent. Exemplary covalent chemistries include, but are not limited to, click chemistries or chemistries set forth in U.S. Pat. Nos. 11,203,612 or 11,505,796; or US Pat. App. Pub. No. 2022/0162684 A1, each of which is incorporated herein by reference. Another example is the SpyTag/SpyCatcher system (See, Zakeri et al. Proceedings Nat'l Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The SpyCatcher can function by irreversibly bonding to a SpyTag through an isopeptide bond. Any of a variety of non-covalent bonds can be used to attach an analyte, affinity reagent, docker, tether or other moiety to a solid support or particle (e.g., structured nucleic acid particle). Receptors and their ligands can be particularly useful. Examples include, but are not limited to, antibodies, antigens, (strept) avidin (or analogs thereof), biotin (or analogs thereof), affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach a functional moiety to a solid support or particle (e.g., structured nucleic acid particle). Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Reagents and techniques that can be used to non-covalently attach an affinity reagent or other moiety to a particle (e.g., structured nucleic acid particle) are set forth in U.S. Pat. Nos. 11,203,612 or 11,505,796; or US Pat. App. Pub. No. 2022/0162684 A1, each of which is incorporated herein by reference.

For a method that utilizes first and second affinity reagents, the second affinity reagents can recognize the same epitopes or molecules as the first affinity reagents. For example, the second affinity reagents can include the same paratope(s) as the first affinity reagents. Alternatively, the second affinity reagents, although recognizing the same epitopes or molecules as the first affinity reagents, can have a different composition compared to the first affinity reagents, for example, having different paratope(s) compared to the paratope(s) present in the first affinity reagents. In some configurations of the methods, the second affinity reagents can recognize different epitopes or molecules from the first affinity reagents. Optionally, the second affinity reagents and first affinity reagents can recognize the same epitope(s) or molecule(s) but with different affinities. For example, the second affinity reagents can differ from the first affinity reagents with regard to K_{D}, k_{off}, kₒₙ or binding probability for a given epitope or set of epitopes.

The first and second affinity reagents can have the same labels and/or tethers. This can be the case independent of the similarity or difference of the paratopes of the first and second affinity reagents. Alternatively, the first and second affinity reagents can differ with respect to their labels and/or tethers. Again, this can be the case independent of the similarity or difference of the paratopes of the first and second affinity reagents. Different labels or tethers, whether in number or kind, can allow tuning of the detectable properties of the affinity reagents, for example, to provide for more uniform detector settings or to accommodate a range of affinities for various types of affinity reagents.

For a method that utilizes first and second analytes, the second analytes can recognize the same paratopes as the first analytes. For example, the second analytes can include the same epitope(s) as the first analytes. Alternatively, the second analytes, although recognizing the same paratopes as the first analytes, can have a different composition compared to the first analytes, for example, having different epitopes(s) compared to the epitopes(s) present in the first affinity reagents. In some configurations of the methods, the second analytes can recognize different paratopes from the first analytes. Optionally, the second analytes and first analytes can recognize the same paratopes(s) but with different affinities. For example, the second analytes can differ from the first analytes with regard to K_{D}, K_{off}, kₒₙ or binding probability for a given paratope or set of paratopes.

A method, composition, or system set forth herein may utilize a secondary detection reagent. In an aspect, provided herein is a method, comprising: (a) providing a solid support comprising an address, wherein the address is attached to an analyte, (b) coupling a first affinity reagent to the analyte, wherein the affinity reagent is not coupled to a detectable label, (c) after coupling the first affinity reagent to the analyte, coupling a second affinity reagent to the first affinity reagent, wherein the second affinity reagent is coupled to the detectable label, and (d) detecting a signal from the detectable label at the address of the solid support. In another aspect, provided herein is a composition, comprising: (a) a solid support comprising a plurality of sites, wherein the plurality of sites is attached to a plurality of analytes, and wherein each site of the plurality of sites is attached to only one analyte of the plurality of analytes, (b) a plurality of first affinity reagents coupled to analytes of the plurality of analytes, wherein each individual first affinity reagent is not coupled to a detectable label, and (c) a plurality of second affinity reagents coupled to the plurality of first affinity reagents, wherein only one second affinity reagent is coupled to each individual first affinity reagent, and wherein each second affinity reagent is coupled to a detectable label. In another aspect, provided herein is a system, comprising: (a) a solid support comprising a plurality of sites, wherein the plurality of sites is attached to a plurality of analytes, and wherein each site of the plurality of sites is attached to only one analyte of the plurality of analytes, (b) a plurality of first affinity reagents, wherein each individual first affinity reagent is not coupled to a detectable label, and wherein each first affinity reagent has a binding specificity for analytes of the plurality of analytes, and (c) a plurality of second affinity reagents, wherein each second affinity reagent is coupled to a detectable label, and wherein each second affinity reagent has a binding specificity for a first affinity reagent of the plurality of first affinity reagents.

A secondary detection reagent may be provided according to any binding reagent composition set forth herein. A secondary detection agent can comprise any binding reagent that is configured to bind to another binding reagent rather than an analyte. A system or composition may contain a primary detection reagent that is configured to bind to an analyte, and a secondary detection reagent that is configured to bind to the primary detection reagent. In some configurations, a secondary detection reagent may comprise a retaining component, a detectable label, and/or a tether strand. A secondary detection reagent may be provided as a multivalent affinity reagent as set forth herein (e.g., comprising two or more affinity reagents). In some configurations, a primary detection reagent may be substantially devoid of a detectable label (e.g., a fluorophore, a luminophore, a spin label, a radiolabel, etc.). In some configurations, a primary detection reagent may comprise an affinity reagent without an additional attached component (e.g., not attached to a retaining component, a detectable label, or a tether strand). Alternatively, a primary detection agent may comprise a non-optical label, such as an affinity tag or a purification tag. Accordingly, a secondary detection agent may comprise an affinity reagent that is configured to bind to the non-optical label. In some configurations, a primary detection reagent may comprise a first antibody having a binding specificity for an analyte, and a secondary detection reagent may comprise a second antibody having a binding specificity for the first antibody. The skilled person will readily recognize that a first affinity reagent of a primary detection agent can comprise any type of affinity reagent set forth herein, and a secondary detection reagent can comprise any type of affinity reagent that is configured to bind to a moiety of the primary detection reagent.

A method that utilizes a secondary detection reagent may comprise a step of delivering a secondary detection reagent to a system comprising a primary detection reagent. In some cases, a primary detection reagent and a secondary detection reagent may be simultaneously delivered to a system or composition set forth herein. In other cases, a primary detection reagent and a secondary detection reagent may be sequentially delivered to a system or composition set forth herein. For example, a plurality of primary detection reagents may be delivered to an array comprising a plurality of analytes, thereby binding primary detection reagents to analytes of the array of analytes. Subsequently, unbound primary detection reagents may be rinsed from the array of analytes by a rinsing medium comprising a plurality of secondary detection reagents.

Systems and methods of the present disclosure may utilize binding reagents that are configured to bind to more than one unique analyte. For example, methods and system may utilize promiscuous binding reagents that bind to more than one structurally-unique protein of a proteome. Because the desired binding reagents bind to a plurality of targets, there may exist a range of binding affinities for binding interactions between a binding reagent and its multiple potential binding targets. Accordingly, a primary detection reagent may be provided at an increased concentration, thereby increasing a likelihood that the primary detection reagent binds to binding targets to which it has a lower affinity. A secondary detection reagent may be selected with a higher affinity for a primary detection reagent than the primary detection reagent has for its binding target. Accordingly, a secondary detection reagent may be provided at a lower concentration than the primary detection reagent.

In some cases, a ratio of a concentration at which a primary detection reagent is provided to a concentration at which a secondary detection reagent is provided may be at least about 0.1, 0.5, 1, 1.1, 1.5, 2, 5, 10, 20, 50, 100, 500, 1000, or more than 1000. Alternatively or additionally, a ratio of a concentration at which a primary detection reagent is provided to a concentration at which a secondary detection reagent is provided may be no more than about 1000, 500, 100, 50, 20, 10, 5, 2, 1.5, 1.1, 1, 0.5, 0.1, or less than 0.1.

One or more steps of a method set forth herein can be repeated at least 1, 2, 3, 4, 5, 10, 50, 100, 200, 300, 400, 500 or more times. Alternatively or additionally, one or more steps of a method set forth herein can be repeated at most 500, 400, 300, 200, 100, 50, 10, 5, 4, 3, 2 or 1 times. Some or all of the repetitions can be carried out using different affinity reagents and/or different analytes from one cycle to the next. Some or all of the repetitions can be carried out using affinity reagents and/or analytes having the same function from one cycle to the next. Some or all of the repetitions can be carried out using affinity reagents and/or analytes having the same composition from one cycle to the next. Some or all of the repetitions can be carried out using different conditions from one cycle to the next. Exemplary conditions that can differ include, but are not limited to, the composition of labels attached to affinity reagents or analytes, concentration of affinity reagents in fluid phase, concentration of analytes in fluid phase, quantity of affinity reagents in fluid phase, quantity of analytes is fluid phase, duration for one or more step in the cycle, temperature for one or more step in the cycle, ionic strength for one or more step in the cycle, pH for one or more step in the cycle, detector gain or sensitivity for one or more step in the cycle, presence or concentration of denaturants for one or more step in the cycle, or presence or one or more reagent set forth herein for one or more step in the cycle.

A multicycle method can include a step of removing first affinity reagents and then delivering second affinity reagents. In some configurations, a multicycle method can include a step of removing first analytes and then delivering second analytes. Typically, removal of affinity reagents or analytes is performed after a detection step. However, in some cases it may be desirable to remove affinity reagents or analytes prior to detection. Optionally, a removal step can be followed by one or more wash steps, wherein a wash fluid is used to remove residual analytes or affinity reagents left behind by the removal step.

### Additional Methods of Proximity-Based Detection

Methods set forth herein may comprise a step of recording a binding interaction between an affinity reagent and an analyte. Recording of a binding interaction may occur during an equilibration period between a plurality of affinity reagents and a plurality of analytes. Preferably, the equilibration period may occur for a sufficient time to establish a binding equilibrium between the affinity reagents and the analytes. A method of recording a binding interaction between an affinity reagent and an analyte may comprise forming a reaction, in which the reaction facilitates detection of a signal associated with a complex containing an analyte and an affinity reagent after an equilibration period has ended. Preferably, binding interactions May be recorded in a spatially resolved manner such that a binding interaction between a first analyte and a first affinity reagent can be resolved from a binding interaction between a second analyte and a second affinity reagent. Array-based methods set forth herein may be especially useful for resolving binding interactions between pluralities of analytes and pluralities of affinity reagents.

While numerous methods are exemplified by a signal becoming detectable when a binding interaction occurs, many described methods can easily be reversed to have a signal become undetectable when a binding interaction occurs (e.g., via removal or dissociation of a detectable label, etc.).

A method may comprise direct recording of a binding interaction between an affinity reagent and an analyte if a reaction of a first moiety co-localized with the affinity reagent with a second moiety co-localized with the analyte inhibits dissociation of the affinity reagent from the analyte, thereby facilitating detection of a signal associated with a complex containing the affinity reagent and the analyte. A method may comprise indirect recording of a binding interaction if a detectable label is retained with the affinity reagent or analyte after the affinity reagent has dissociated from the analyte, thereby facilitating detection of a signal associated with a complex containing the affinity reagent and analyte after the complex has dissociated.

In some cases, a transferrable moiety may be attached to a transferring moiety by an irreversible non-dissociable interaction. Exemplary irreversible non-dissociable binding interactions can include covalent binding interactions (e.g., Click-type reactions) and receptor-ligand binding interactions (e.g., streptavidin-biotin, SpyCatcher-SpyTag, SnoopCatcher-SnoopTag, SdyCatcher-SdyTag, etc.). For methods having multiple possible detection events, it may be preferable to have a receiving transferring moiety be configured to be releasable so that the signal associated with transfer of the transferrable moiety can be erased after a detection event by release of the transferrable moiety-transferring moiety complex. For example, a non-dissociable attachment moiety may be attached to a first oligonucleotide, in which the first oligonucleotide is hybridized to a second oligonucleotide, and in which the second oligonucleotide is attached to a solid support. Accordingly, the first oligonucleotide can be released (e.g., by a toehold-mediated strand displacement reaction, by contacting with a chaotrope, by heating, etc.), thereby releasing any transferrable moiety attached to the first oligonucleotide.

FIGS. 2A-2H depict various methods of recording a binding interaction by polymerization or depolymerization. Such methods can utilize a polymerizing agent (e.g., a polymerizing enzyme, a chemical reactant, etc.) or a depolymerizing agent (e.g., a depolymerizing enzyme, a chemical reactant, etc.) to add or remove residues from a polymeric chain, respectively. Polymerization or depolymerization can occur in an iterative fashion (e.g., via the sequential addition or removal of single residues or monomers) or in a step-wise fashion (e.g., by the simultaneous addition or removal of multiple residues).

FIGS. 2A-2D depict a method of recording a binding interaction by modifying a modifiable moiety 206 with a polymerizing enzyme (e.g., terminal deoxynucleotidyl transferase) that iteratively adds monomers to the modifiable moiety 206. FIG. 2A depicts in an initial configuration an analyte 210 that is immobilized at a fixed address of a solid support 200. A modifiable moiety 206 (e.g., an oligonucleotide, a peptide, a polymer chain, etc.) is immobilized at the fixed address of the solid support 200 containing the immobilized analyte 210. In some cases, the modifiable moiety may be releasably attached to the solid support 200. For example, the modifiable moiety 206 may comprise an oligonucleotide that is coupled to a complementary oligonucleotide 205, in which the complementary oligonucleotide is coupled to the solid support. Preferably, the modifiable moiety 206 is co-localized at a distance from the immobilized analyte 210 that is not optically-resolvable. An affinity reagent 220 is bound to the immobilized analyte 210, thereby immobilizing the affinity reagent 220 at the fixed address of the solid support 200. The affinity reagent 220 is attached to a linking moiety 221 that is also attached to a polymerizing agent 222 (e.g., TdT enzyme). FIG. 2B depicts a second configuration, in which the polymerizing enzyme has attached a sequence of residues to the modifiable moiety while bound to the analyte 210. For the configuration utilizing the TdT enzyme, the modifiable moiety would be modified with a poly-T nucleotide sequence 208. As shown in FIG. 2B, the affinity reagent 220 has dissociated from the analyte 210.

FIGS. 2C-2D depict alternative detection methods for detecting the recoded binding interaction between the analyte 210 and the affinity reagent 220. FIG. 2C depicts hybridization of a poly-A oligonucleotide 208B to the poly-T oligonucleotide 208A formed by the polymerization agent 222. If the oligonucleotide 208B comprises a detectable label 209, a signal from the detectable label 222 can be detected at an address containing the analyte 210. Alternatively, FIG. 2D depicts further modification of the modifiable moiety to also record information regarding the address where the modification moiety was modified. In the depicted configuration, the modifiable moiety is attached to by a capture sequence 206A to a complementary capture sequence 205A of the complementary oligonucleotide. The complementary oligonucleotide further comprises a barcode sequence 205B that is unique to the address containing the analyte 210 (i.e., each individual analyte would be co-localized with a barcode sequence that is unique from all other barcode sequences). The modifiable moiety can be extended, for example by a polymerase extension reaction, thereby attaching a complementary barcode sequence 206B to the modifiable moiety. The modifiable moiety can be released and sequenced, thereby detecting the poly-T extension 208 attached to the complementary barcode sequence 206B. Accordingly, the binding interaction between the affinity reagent 220 and the analyte 210 can be detected by the detection of the modifiable moiety.

FIG. 2E depicts the system depicted in FIGS. 2A-2D in a multi-analyte format. The left modifiable moiety 206 associated with analyte 210 has a longer poly-T extension 208C than the poly-T extension 208D of the modifiable moiety 206 associated with analyte 211. The increased length of poly-T extension 208C relative to poly-T extension 208D may be due to increased retention of an affinity reagent 220 containing a polymerization agent 222 with analyte 210 relative to analyte 211. Accordingly, detecting the length of a modifiable moiety 206 modified by a polymerization or depolymerization agent may provide information on the kinetics of the interaction between the affinity reagent and the analyte. Such information may facilitate identification or characterization of the analyte to which the affinity reagent is observed to bind.

FIGS. 2F and 2G depict systems utilizing polymerization agents other than TdT enzyme. As shown in FIG. 2F, an affinity reagent may be linked to a polymerase enzyme 223. The polymerase enzyme may extend the complementary oligonucleotide 205 with a sequence from the modifiable moiety 206 (as shown in the right-side configuration). Accordingly, the complementary oligonucleotide can be released and sequenced, thereby detecting a sequence of the modifiable moiety 206 attached to the complementary oligonucleotide 205. Preferably, the complementary oligonucleotide 205 may comprise a unique barcode sequence that is associated with the address containing analyte 210. FIG. 2G depicts a system containing a ligase enzyme 224 attached to the affinity reagent 220. The modifiable moiety 206 may be hybridized to a recoding sequence 211 that is held in proximity to the complementary oligonucleotide 205 by the modifiable moiety 206. The ligase enzyme 224 can attach the recoding sequence 211 to the complementary oligonucleotide 205, thereby recoding the binding interaction of the affinity reagent 220 with the analyte 210. The right-side configuration of FIG. 2G depicts a subsequent detection configuration, in which a detectable oligonucleotide 212 that is at least partially-complementary to the complementary oligonucleotide 205 and the recording sequence 211. The detectable oligonucleotide 212 can comprise a detectable label 209 that provides a signal at the address containing the analyte 210.

FIG. 2H depicts an affinity reagent 220 attached to a depolymerization agent 225 (e.g., an exonuclease, a restriction enzyme, a protease, etc.). The depolymerization agent 225 can remove one or more residues from a residue sequence 208 attached to a modifiable moiety 206. The right-hand configuration depicts a configuration after the depolymerization agent has been co-localized at the address containing the analyte 210 for sufficient time to remove the full poly-T sequence 208. Accordingly, absence of a signal from the address containing analyte 210 may facilitate detection of the binding interaction between the affinity reagent 220 and the analyte 210. For example, a poly-A oligonucleotide with a detectable label 209 may only be bound at addresses containing intact poly-T sequences due to absence of affinity reagent binding.

In an aspect, provided herein is a method, comprising: (a) providing a solid support comprising an address, wherein the address is attached to an analyte and a polymer strand, (b) coupling an affinity reagent to the analyte at the address, wherein the affinity reagent is attached to a polymerization agent or a depolymerization agent, (c) after coupling the affinity reagent to the analyte, altering the polymer strand with the polymerization agent or depolymerization agent, and (d) after altering the polymer strand, detecting the altered polymer strand, thereby detecting the coupling of the affinity reagent to the analyte. In another aspect, provided herein is a method of distinguishing a first analyte from a second analyte, comprising: (a) providing a solid support comprising a first address and a second address, wherein the first address is attached to a first analyte and a first polymer strand, and wherein the second address is attached to a second analyte and a second polymer strand, (b) contacting affinity reagents to the solid support, wherein each affinity reagent is attached to a polymerization agent or a depolymerization agent, (c) after contacting the affinity reagent to the solid support, detecting a first characteristic of the first polymer strand, and detecting a second characteristic of the second polymer strand, and (d) based upon the first characteristic of the first polymer strand and the second characteristic of the second polymer strand, distinguishing the first analyte from the second analyte.

In another aspect, provided herein is a composition, comprising: (a) a solid support comprising a plurality of sites, wherein the plurality of sites is attached to a plurality of analytes, wherein each site of the plurality of sites is attached to only one analyte of the plurality of analytes, and wherein each site of the plurality of sites is attached to a polymer strand, (b) a plurality of affinity reagents coupled to analytes of the plurality of analytes, wherein each individual affinity reagent is attached to a polymerization agent or a depolymerization agent, and (c) a fluidic medium contacted to the solid support, wherein the fluidic medium comprises a plurality of residues, wherein residues of the plurality of residues are a substrate for or a product of the polymerization agent or depolymerization agent. In another aspect, provided herein is a system, comprising: (a) a solid support comprising a plurality of sites, wherein the plurality of sites is attached to a plurality of analytes, and wherein each site of the plurality of sites is attached to only one analyte of the plurality of analytes, and wherein each site of the plurality of sites is attached to a polymer strand, (b) a first fluidic medium comprising a plurality of affinity reagent, wherein each individual affinity reagent is attached to a polymerization agent or a depolymerization agent, (c) a fluidic system, wherein the fluidic system is configured to deliver the first fluidic medium to the solid support, and (d) a detection device, wherein the detection device is configured to detect a polymer strand altered by the polymerization agent or depolymerization agent.

A binding reagent may be provided to a method, composition, or system set forth herein, in which the binding reagent comprises an affinity reagent attached to a polymerization agent or a depolymerization agent. A binding reagent may provide a detectable signal by altering a polymer strand associated with an analyte when the binding reagent binds to an analyte associated with the polymer strand. For example, an analyte may be associated with a nucleic acid polymer strand, and the nucleic acid polymer strand may be altered by a polymerization agent comprising a polymerase, a terminal deoxynucleotidyl transferase, and endonuclease, or a nucleic acid ligase, or may be altered by a depolymerization agent comprising an exonuclease or a restriction enzyme. In another example, an analyte may be associated with a peptide polymer strand, and the peptide polymer strand may be altered by a polymerization agent comprising a peptide ligase or a ribosome, or the peptide polymer strand may be altered by a depolymerization agent comprising a protease. In another example, an analyte may be associated with a polysaccharide polymer strand, and the polysaccharide polymer strand may be altered by a polymerization agent comprises a glycosyltransferase, or the polysaccharide polymer strand may be altered by a depolymerization agent comprising a glycosidase.

A method of utilizing a binding reagent comprising a polymerization agent or a depolymerization agent may comprise attaching a plurality of residues or monomers to the polymer strand with the polymerization agent. Attaching the plurality of residues or monomers may occur in the presence of a fluidic medium comprising the plurality of residues or monomers. Accordingly, coupling a binding reagent to an analyte may comprise delivering to a solid support a fluidic medium comprising the affinity reagent and the plurality of residues or monomers. In some cases, coupling a binding reagent to an analyte may comprise the steps of: i) delivering to the solid support a first fluidic medium comprising the affinity reagent, and ii) after delivering the first fluidic medium, delivering a second fluidic medium comprising the plurality of residues or monomers.

In some cases, a polymerization agent may perform a step-wise extension of a polymer strand. Altering the polymer strand can comprise simultaneously attaching a plurality of residues or monomers to the polymer strand with the polymerization agent. For example, a ligase enzyme may attach a first polymer strand to a second polymer strand. In other cases, a polymerization agent may perform single-residue extension of a polymer strand. Altering the polymer strand can comprise sequentially attaching each residue or monomer of a plurality of residues or monomers to the polymer strand with the polymerization agent. For example, a TdT enzyme will serially attach a single nucleotide to a single-stranded nucleic acid.

A method of utilizing a binding reagent comprising a polymerization agent or a depolymerization agent may comprise detaching a plurality of residues or monomers from the polymer strand with a depolymerization agent. In some cases, altering the polymer strand can comprise simultaneously detaching a plurality of residues or monomers from the polymer strand with the depolymerization agent. For example, a protease may cleave a peptide strand from a longer peptide strand. In other cases, altering the polymer strand comprises sequentially detaching a plurality of residues or monomers from the polymer strand with the depolymerization agent. For example, an exonuclease may excise a single nucleotide from a nucleic acid strand.

A polymer strand altered by a polymerization agent or a depolymerization agent may be detectable by contacting a detectable probe to the polymer strand. A method may further comprise: i) binding a detectable probe to a plurality of residues or monomers attached to the polymer strand, and ii) detecting a signal from the detectable probe at the address of the solid support. For example, labeled poly-A oligonucleotides may be utilized as probes to detect polymer strands provided poly-T extensions by a binding reagent comprising a TdT enzyme. Likewise, a labeled antibody can be utilized to detect a peptide affinity tag attached by a binding reagent comprising a peptide ligase. The skilled person will recognize that the activity of a depolymerization agent may render a polymer strand undetectable, therefore presence of a signal at an address can be interpreted as absence of binding of a binding reagent containing the depolymerization agent at the address. Alternatively, a method may comprise detecting an absence of a signal at the address of the solid support. An absence of signal at an address may arise due to the removal of a detectable label from a polymer strand by a depolymerization, or an absence of binding of a binding reagent containing a polymerization agent at the address.

Alteration of polymer strands associated with analytes by binding reagents, as set forth herein, may facilitate distinguishing of analytes of a plurality of analytes. Presence or absence of a signal associated with an alteration of a polymer strand may be indicative of whether a binding reagent was present at an address containing the polymer strand, and optionally may be indicative of how long the binding reagent was bound at the address. A method may comprise detecting characteristics of polymer strands, thereby determining presences or absences of binding of binding reagents at the addresses containing the polymer strands.

In some cases, a first characteristic of a first polymer strand or detecting a second characteristic of a second polymer strand can comprise detecting a residue sequence of the first polymer strand or the second polymer strand. In particular cases, detecting the first characteristic of the first polymer strand and detecting the second characteristic of the second polymer strand can comprise detecting a presence of the residue sequence in the first polymer strand and detecting an absence of the residue sequence in the second polymer strand. Presence or absence of a residue sequence may be detected by a method set forth herein, such as utilizing nucleic acid probes or binding of affinity reagents to an altered residue sequence of a polymer strand. Alternatively, a sequencing device may be utilized to detect presence or absence of a residue sequence of a polymer strand.

In some cases, detecting a first characteristic of a first polymer strand or detecting a second characteristic of a second polymer strand can comprise detecting a length of a residue sequence of the first polymer strand or the second polymer strand. In particular cases, detecting the first characteristic of the first polymer strand and detecting the second characteristic of the second polymer strand can comprise detecting a difference in length between the length of the residue sequence of the first polymer strand and the length of the residue sequence of the second polymer strand. A sequencing device may be utilized to detect a length of a residue sequence of a polymer strand. Length of a polymer strand may be indicative of a length of a time period during which a binding reagent remained bound. For example, if a binding reagent binds to a first analyte for a longer time period than it binds to a second analyte, a residue sequence of a polymer strand associated with the first analyte may be longer or shorter than a residue sequence of a polymer strand associated with the second analyte.

Methods and systems of the present disclosure set forth systems of dockers and tethers that may be useful for inhibiting dissociation of an affinity reagent from an analyte after the affinity reagent has become bound to the analyte. Oligonucleotide-based docker/tether systems may be particularly useful for methods and systems set forth herein. In some cases, it may be advantageous to provide an oligonucleotide in a fluid phase that is configured to bind to a docker nucleic acid strand and a tether nucleic acid strand.

Certain methods set forth herein for recording a binding interaction between an affinity reagent and an analyte may be amenable to a method of kinetic control. Kinetic control may comprise any suitable method for controlling the rate of a secondary binding reaction that records the binding interaction between the affinity reagent and the analyte. Preferably, the binding on-rate for the reaction is slower than the binding on-rate for the affinity reagent with the analyte, thereby limiting the likelihood of a false detection event. It will be recognized that an affinity reagent may have a known and/or characterized tendency to bind to certain epitopes or analytes other than its primary target (i.e., off-target binding). Off-target binding may be characterized by a faster off-rate of the affinity reagent from an analyte having a secondary binding target relative to its off-rate from an analyte having a primary binding target for the affinity reagent. In such cases, it may be preferable for a reaction to have a binding on-rate that is slower than the binding off-rate for an affinity reagent with a secondary binding target. Accordingly, the reaction will be more likely to record binding interactions between an affinity reagent and its primary binding target.

It will be understood that methods and systems exemplified herein via a single analyte and a single affinity reagent can readily be expanded into methods and system containing pluralities of analytes and/or affinity reagents. Array-based methods may be especially useful for observing binding interactions between pluralities of analytes and pluralities of affinity reagents at single-analyte resolution. For some array-based methods, particles (e.g., nucleic acid nanoparticles) may be useful for co-localizing certain components at a single fixed address of the array. For example, a single analyte and a docker strand, as set forth herein, may be attached to a single nucleic acid nanoparticle, in which the nucleic acid nanoparticle is attached to a fixed address of a solid support. Accordingly, each individual address of a plurality of addresses of a single-analyte array may comprise a single particle, in which the single particle is attached to one and only one analyte, and at least one other moiety that is configured to facilitate formation of a reaction, as set forth herein.

It will be further understood that methods and systems exemplified by a single set of moieties configured to form a reaction may further comprise additional moieties configured to form reactions. For example, an analyte may be co-localized with a plurality of docker strands, and/or an affinity reagent may be attached to a plurality of tether strands. In another example, a linking moiety may comprise a plurality of FRET dyes that can be brought into proximity of a plurality of complementary FRET dyes.

### Assaying Proteins

Any of a variety of assay formats can be used to detect an analyte. Several methods will be exemplified below in the context of detecting proteins but can readily be extended to other analytes by modifications that will be apparent to those skilled in the art. Assay methods and formats set forth below can be modified to include linked affinity reagents (or linked analytes) or to apply various stimuli to attract affinity reagents (or analytes) as set forth above.

A protein can be detected using one or more affinity reagents having binding affinity for the protein. The affinity reagent and the protein can bind each other to form a complex and the complex can be detected during or after formation. The complex can be detected directly, for example, due to a label that is present on the affinity reagent or protein. In some configurations, the complex need not be directly detected, for example, in formats where the complex is formed and then the affinity reagent, protein, or a label component that was present in the complex is subsequently detected.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers can be expected to occur in a substantial number of different proteins in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g., the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g., amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g., affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g., a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g., addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patents application Ser. Nos. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g., single protein), a single complex of two or more molecules (e.g., a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

A method of the present disclosure can be performed in a multiplex format. In multiplexed configurations, different analytes can be attached to different unique identifiers (e.g., proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events.

A particularly useful multiplex format uses an array of analytes (e.g., proteins) and/or affinity agents. The analytes and/or affinity agents can be attached to unique identifiers (e.g., addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g., ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e., an ensemble of the analytes). Alternatively, an address can include a population of different analytes.

A detection apparatus can include a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g., luminescent detection), one or more analytes (e.g., proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide assisted excitation, or the like. Optical filters or other optical components can be present to tune the wavelength, polarization or other optical properties of excitation and/or emission radiation used by a luminescence detector.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

A light sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Selective excitation of analytes immobilized in wells may facilitate single-molecule detection in the presence of unbound detectable probes. A zero-mode waveguide can be fabricated by patterning wells into a solid layer (e.g., an optically opaque material) that is disposed on an optically transmitting material (e.g., silica, quartz, fused silica, borosilicate glass, etc.). Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

A detection apparatus need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e., one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 micron, or more than 1 micron. Alternatively or additionally, analyte-binding sites may have an average characteristic dimension of no more than about 1 micron, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support. Compositions set forth herein can interact with each other via covalent bonds.

Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium May comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid. A method set forth herein can include a step of adding a post-translational modification to a protein by contacting the protein with an enzyme (e.g., a kinase) or other modifying agent.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g., sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g., histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylglucosaminidase can be used to cleave non-reducing terminal β-linked N-acetylglucosamine from proteins; endo-a-N-acetylgalactosaminidase can be used to remove O-glycosylation, for example, removing serine- or threonine-linked unsubstituted Galb 1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.). Useful affinity reagents for proteoform-specific detection can include affinity reagents that bind to an epitope containing a post-translational modification or bind to an epitope specific to an isoform of a protein.

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g., positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654; US Pat. App. Pub. Nos. 2020/0318101 A1 or 2023/0114905 A1, or Egertson et al., BioRxiv (2021), DOI: 10.1101/2021.10.11.463967, each of which is incorporated herein by reference.

One or more compositions set forth herein can be present in an apparatus or vessel. For example, a linker-attached affinity reagent and/or linker-attached analyte of the present disclosure can be present in a vessel, such as a flow cell. In some cases, the linker is attached to the affinity reagent or the analyte via a particle (e.g., structured nucleic acid particle). As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

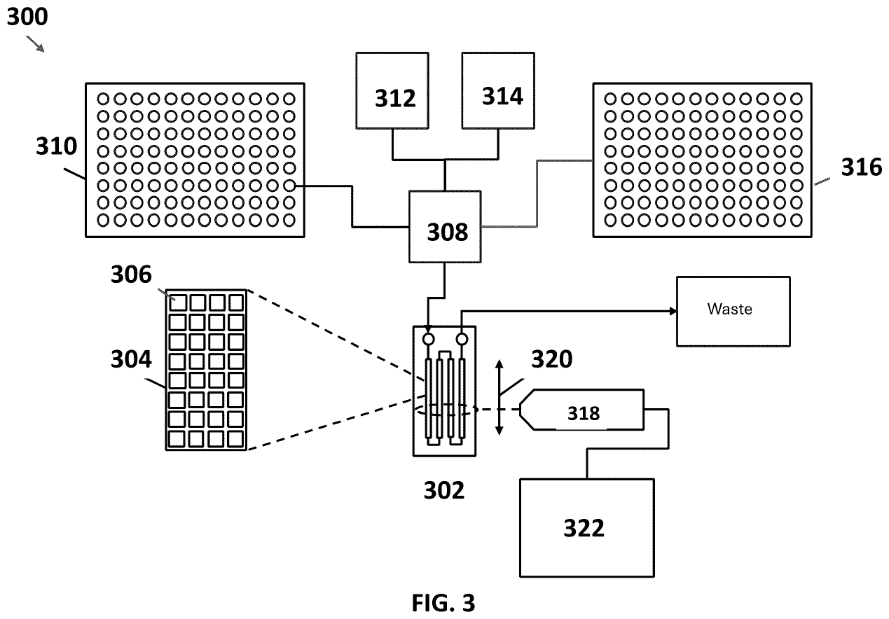

Additionally, provided herein are systems for performing the techniques, reagents, systems, and methods described herein. An example of a system is illustrated in FIG. 3. As shown, the system 300 includes a flowcell 302 that includes an array surface (shown as 304) within the channels of the flow cell upon which individual protein molecules from a sample may be deposited and immobilized in locations 306 that are individually addressable, and in particular cases are individually optically resolvable from each other using, e.g., fluorescence microscopy or scanning techniques.

The system will also typically include a fluidic delivery system 308 that is configured to deliver different fluids to the flow cell 302 through a series of fluidic lines and utilizing appropriate pumps, valves and other conventional fluid controls. The fluidics system 308 may be fluidically coupled to various sources of fluids and reagents needed to carry out the analysis on the flow cell. For example, as shown, fluidic system 308 is fluidly coupled to a source of a plurality of reagents 310 (shown as a 96 well plate, although any number of different reagent storage systems of varying capacity may be employed) that includes a library of multiple affinity reagents that each have affinity for different characteristics of one or more proteins of interest. Additionally, fluidic system 308 may also be coupled to sources of washing fluids or buffers 312, and removal reagents 314 (for removing bound affinity reagents following detection), as well as any other ancillary fluids and reagents needed for the analysis. Similarly, where flow cells are prepared on the system, the fluidic system may be coupled to sources of different sample materials that are to be analyzed 316 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 318, for detecting and recording optical signals (e.g., emitted photons) arising from different positions on the array surface. Such detection systems may generally include line scanning confocal fluorescent microscope systems, which are capable of scanning across large array surfaces (as shown by arrow 320) to detect and record fluorescence across such surfaces at reasonably high scan rates.

The overall systems also typically include one or more computers or processors 322 for controlling the operation of the instrument system including the fluidic system 308 (e.g., to sample different sample sources 316, reagent sources 2010 and delivery timing and volume of each), and detection system 318, among other functions, and for recording the detected signals received from the detection system 318, e.g., fluorescent signals, and analyzing such signals to identify potential binding by each of the different affinity reagents. Processors 322 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been describe in, for example, U.S. Pat. Nos. 11,545,234, 10,473,654B1, and Eggertson, et al., A theoretical framework for proteome-scale single-molecule protein identification using multi-affinity protein binding reagents, bioRxiv, https://doi.org/10.1101/2021.10.11.463967, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 322 can be an electronic device of a detection system, the electronic device being integral to the detection system or remotely located with respect to the detection system. The computer system 322 includes a computer processing unit (CPU, also “processor” and “computer processor” herein), which can be a single core or multi core processor, or a plurality of processors for parallel processing. The computer system 322 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 322 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 322, can implement a peer-to-peer network, which may enable devices coupled to the computer system 322 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 322 can be included in the circuit. In some cases, the circuit is an application specific integrated circuit (ASIC).

The storage unit can store files, such as drivers, libraries and saved programs. The storage unit can store user data, e.g., user preferences and user programs. The computer system 322 in some cases can include one or more additional data storage units that are external to the computer system 322, such as located on a remote server that is in communication with the computer system 322 through an intranet or the Internet.

The computer system 322 can communicate with one or more remote computer systems through the network. For instance, the computer system 322 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 322 via the network.

Methods as described herein can be implemented by way of machine (e.g., computer processor) executable code stored on an electronic storage location of the computer system 322, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 322, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 322 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### EXAMPLES

### Example 1. Preparation of Detectable Probes

Detectable probes against trimer amino acid epitopes were prepared. A first antibody was prepared against the trimer epitope histidine-serine-proline. The antibody was expressed as a human IgG with a V5 peptide tag attached to its C-terminus. A second antibody was prepared against the trimer epitope tryptophan-valine-serine. The antibody was expressed as a human IgG with an HA tag attached to its C-terminus.

Secondary detection moieties were prepared for each of the two detectable probes. An anti-V5 tag IgG antibody was prepared. The anti-V5 tag antibodies were randomly labeled with Alexa-Fluor 532 dyes. An anti-HA tag IgG antibody was prepared. The anti-HA tag antibodies were randomly labeled with Alexa-Fluor 647 dyes. The secondary detection moieties were mixed with the trimer antibodies to form detectable probes. Anti-V5 tag antibodies bound to the V5 tags of the HSP-specific antibodies, and the anti-HA tag antibodies bound to the HA tags of the WVS-specific antibodies.

### Example 2. Detection of Peptides with Multiplexed Detectable Probes

The multiplexed detectable probe mixture described in Example 1 was tested against trimer peptide targets to confirm the detection ability of secondary-labeled antibodies. Detectable probes were tested by a dual-color ELISA assay, with each peptide-containing vessel being illuminated with 532 nanometer (nm) and 647 nm light. Sets of three separate vessels were prepared, with the first vessel of each set containing only the HSP peptide target, the second vessel of each set containing only the WVS peptide target, and the third vessel of each set containing a 1:1 mixture of the HSP and WVS peptide targets. For each set, the respective peptide concentration was uniform amongst each of the three vessels. Peptide concentration was varied amongst sets of vessels, ranging from 0 nanomolar (nM) to 50 nM.

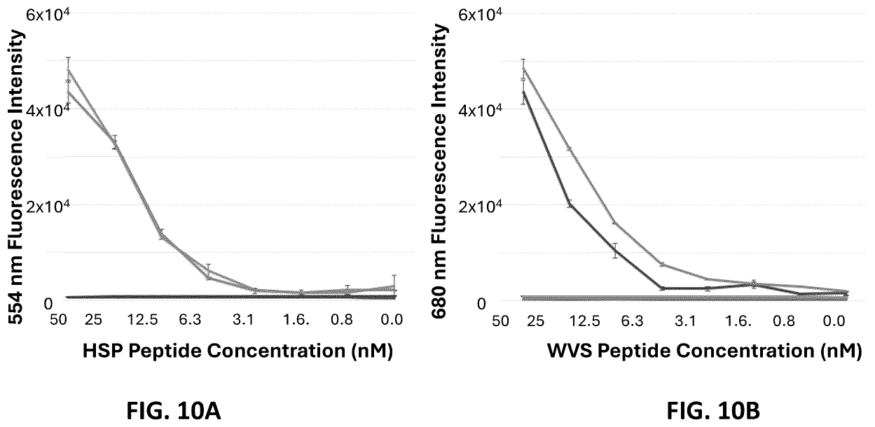

ELISA results are displayed in FIGS. 10A and 10B. FIG. 10A shows detection measurements in the 532 nm excitation channel, corresponding to the measured detection of HSP peptides. There is concordance of measurement between the HSP-only vessel and the HSP/WVS vessel. The WVS-only vessel shows substantially no binding of the HSP-specific detectable probe. FIG. 10B shows detection measurements in the 647 nm excitation channel, corresponding to the measured detection of HSP peptides. There is concordance of measurement between the WVS-only vessel and the HSP/WVS vessel. The HSP-only vessel shows substantially no binding of the WVS-specific detectable probe. The ELISA results suggest that multiplexed affinity reagents labeled by secondary detection moieties could simultaneously detect different epitope targets within the same vessel.

### Example 3. Preparation of Detectable Probes

Detectable probes against dimer and trimer amino acid epitopes were prepared. A first human IgG antibody was prepared against the trimer epitope phenylalanine-phenylalanine-serine. A second human IgG antibody was prepared against the trimer epitope tryptophan-asparagine-lysine. A third human IgG antibody was prepared against the trimer epitope tryptophan-valine-serine. A fourth human IgG antibody was prepared against the dimer epitope tyrosine-phenylalanine.

Secondary detection moieties were obtained from Jackson ImmunoResearch for labeling the human IgG antibodies. Three different secondary detection moieties were tested: (i) anti-Human IgG Fc antibody, (ii) anti-Human IgG Fc Fab #1, (iii) anti-Human IgG Fc Fab #2. Each of the secondary detection moieties were randomly labeled with Alexa Fluor 647 fluorescent dyes. For each of the four primary antibodies, each secondary detection moiety was combined with the primary antibody in a 1:2 ratio of primary antibody to secondary detection moiety. For each of the four primary antibodies, a fourth detectable probe preparation was formed by combining the primary antibody with the secondary detection moiety in a 1:4 ratio. Accordingly, four detectable probe compositions were formed for each of the four primary antibodies.

### Example 4. Detection of Detectable Probes

The detectable probes described in Example 3 were utilized to detect arrays of human transferrin protein. Single-molecule arrays of transferrin were prepared according to methods described in U.S. Pat. Nos. 11,203,612 and 11,505,796, each of which is herein incorporated by reference in its entirety. Briefly, purified samples of transferrin proteins were attached to nucleic acid particles. The protein-nucleic acid conjugates were deposited on a patterned array with an approximately 1 micron site spacing. Each site of the array was configured to bind only one protein-nucleic acid conjugate. Each nucleic acid particle was bound to an array site by conjugation of pendant oligonucleotides of the nucleic acid particle to surface-bound oligonucleotides of the array site. Each nucleic acid particle was labeled with Alexa Fluor 488 dyes. Occupied array sites were determined by fluorescence microscopy, with excitation performed at 488 nm wavelength and emission measured at 519 nm wavelength.

Each detectable probe composition was individually delivered to the single-molecule array. After a detectable probe composition was incubated with the array, unbound detectable probes were rinsed from the array. Subsequently, the array was fluorescently imaged by confocal scanning microscopy. Excitation was performed by laser illumination at 647 nm wavelength and emission was measured at 680 nm wavelength. Co-localization percentage of detectable probes was measured by counting the number of imaged array sites containing both a 519 nm and 680 nm signal divided by the total number of imaged array sites having the 519 nm signal.

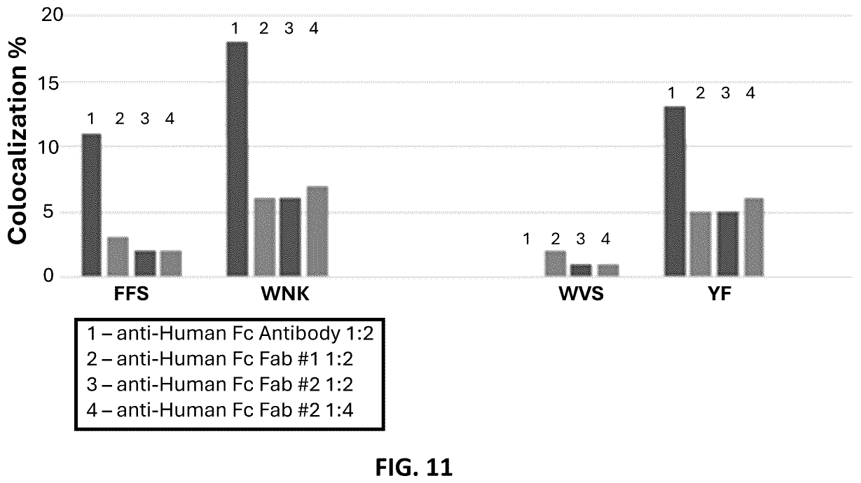

Co-localization data of the detectable probes with transferrin is shown in FIG. 11. For epitopes FFS, WNK, and YF, detectable probes labeled with the IgG antibody (1) showed the highest rates of detection. The labeled Fab detectable probes were observed to produce lower rates of detection for FFS, WNK, and YF than the labeled antibody, and only some improvement was seen by increasing the labeling ratio to 1:4. For detectable probes targeting the WVS epitope, the highest rates of detection were seen for the labeled Fab detectable probes, while minimal detection was observed for the labeled antibody probes.

### Example 5. Cyclical Characterization of Protein Proteoforms

A single-molecule array is formed on a silicon substrate. The array contains proteins from 50 different lysates of brain tissue. Human microtubule-associated protein Tau is isolated and enriched from each lysate sample, then attached to particles at a 1:1 protein:particle ratio. Each particle further contains a sample-specific oligonucleotide barcode, thereby facilitating detection of sites occupied by a particle-protein conjugate. After deposition of proteins on the single-molecule array by attachment of the particles to the array surface, analyte binding sites occupied by proteins from each individual sample are detected by serial hybridization and detection of fluorescently-labeled oligonucleotides complementary to each sample-specific oligonucleotide barcode.

The single-molecule array, containing approximately 109 analyte binding sites, is formed on a silicon substrate. Each analyte binding site is about 100 nanometers (nm) in diameter with an approximately 1 micron spacing between neighboring binding sites. A plurality of oligonucleotides is attached to the silicon at each analyte binding site, with interstitial spaces between analyte binding sites substantially devoid of oligonucleotides. A single particle is immobilized at each analyte binding site by hybridization of oligonucleotides attached to the particle to oligonucleotides attached to the silicon.

The single-molecule array is cyclically contacted with a pool of affinity reagents. Each pool of affinity reagents contains labeled affinity reagents each having the same binding specificity. Eleven different pools of affinity reagents are utilized, with 5 pools containing affinity reagents specific to differing splice isoforms of Tau protein, and 6 pools containing antibodies specific to differing phosphorylated epitopes of Tau protein. The eleven utilized affinity reagents are listed in Table I below. Each of the 11 unique affinity reagents affinity reagent is cycled 3 times for a total of 33 cycles of affinity reagent binding. Affinity reagents from each pool of affinity reagents are human IgG antibodies labeled with an anti-human IgG secondary antibody that is attached to Alexa Fluor 647 fluorescent labels.

TABLE I

| Isoform-Specific | Phosphorylation-Specific |
|---|---|
| Anti-0N | Anti-pT181 |
| Anti-1N | Anti-pS202 |
| Anti-2N | Anti-pT205 |
| Anti-3R | Anti-pS214 |
| Anti-4R | Anti-pT217 |
| | Anti-pT231 |

Each cycle of the protein characterization assay involves the steps of: (1) delivering a pool of affinity reagents to the single-molecule array, (2) incubating the affinity reagents with the immobilized proteins for one hour, (3) after incubation, rinsing unbound affinity reagents from the array, (4) after rinsing unbound affinity reagents, illuminating the array with 647 nm light, (5) detecting on an optical detector a presence or absence of a fluorescent signal from both of the different detectable labels at each analyte binding site, (6) transmitting the signal information from the optical detector to a processor, (7) adding the signal information for each analyte binding site to a binding profile for the protein immobilized at the analyte binding site, and (8) dissociating bound detectable probes from proteins and rinsing all detectable probes from the single-molecule array.

Signal information from all 3 rounds of each of the 11 different affinity reagents is stored in a binding profile contained on the processor. The binding profile is provided to a characterization algorithm that utilizes methods of analyte decoding described in U.S. Pat. Nos. 10,473,654 and 11,282,585, and U.S. patent application Ser. No. 19/342,315. The characterization algorithm determines for each nanowell a most probable protein isoform and the presence of 0 to 6 different phosphorylations on each protein. Protein characterizations are sorted according to which of the 50 samples each protein belongs. For each of the 50 lysate samples, a detected quantity of each possible proteoform is calculated.

### Example 6. Cyclical Characterization of Protein Identities

A single-molecule array is formed on a substrate containing nanowells. The substrate comprises a fused silica solid support with a layer of aluminum deposited onto a surface of the fused silica. Nanowells of approximately 100 nm diameter are formed in the aluminum layer with an approximately 1 micron spacing between neighboring wells. Each nanowell passes through the entire thickness of the aluminum, thereby exposing a region of the surface of the fused silica in the bottom of the nanowell. The surface of the fused silica exposed in the bottom of each nanowell is attached to a plurality of oligonucleotides. A single particle is immobilized in each nanowell by hybridization of oligonucleotides attached to the particle to oligonucleotides attached to the fused silica. A single protein is attached to each particle in the nanowell. The proteins attached to the particles in the nanowells are isolated from a human blood sample.

The single-molecule array is cyclically contacted with sets of affinity reagents. Each set of affinity reagents is multiplexed with respect to binding specificity, with each set containing at least a first type of affinity reagent that differs from a second type of affinity reagent of the set with respect to binding specificity. In each multiplexed set of affinity reagents, the affinity reagents of the first binding specificity are attached to Alexa Fluor 532 fluorescent labels and the affinity reagents of the second binding specificity are attached to Alexa Fluor 647 fluorescent labels, thereby facilitating differentiation of probe binding by differing emission signals. The affinity reagents having the first binding specificity are human IgG antibodies labeled via an anti-human IgG antibody fragment that is attached to the Alexa Fluor 532 labels. The affinity reagents having the second binding specificity are rabbit IgG antibodies labeled via an anti-rabbit IgG antibody fragment that is attached to the Alexa Fluor 647 labels. Prior to delivery to the single-molecule array, each set of multiplexed affinity reagents is stored in a separate vessel containing the differing affinity reagents and their respective labeling Fabs. Each different affinity reagent has a binding specificity for a different dimer, trimer, or tetramer amino acid sequence that is common to multiple different proteins of the human proteome.

Each cycle of the protein characterization assay involves the steps of: (1) delivering a set of multiplexed affinity reagents to the single-molecule array, (2) incubating the affinity reagents with the immobilized proteins for one hour, (3) illuminating each nanowell with 532 nm and 647 nm light in the presence of bound and unbound affinity reagents, (4) detecting on an optical detector a presence or absence of a fluorescent signal from both of the different detectable labels from each nanowell, (5) transmitting the signal information from the optical detector to a processor, (6) adding the signal information for each nanowell to a binding profile for the protein immobilized in the nanowell, and (7) dissociating bound detectable probes from proteins and rinsing all detectable probes from the single-molecule array.

100 cycles (200 different affinity reagents in total) are utilized during the assay. Signal information from each of the 200 different affinity reagents is stored in a binding profile contained on the processor. The binding profile is provided to a characterization algorithm that utilizes methods of analyte decoding described in U.S. Pat. Nos. 10,473,654 and 11,282,585, and U.S. patent application Ser. No. 19/342,315. The characterization algorithm determines for each nanowell a most probable protein identity based upon the signal information for that nanowell.

1. A method, comprising:
(a) delivering a fluidic medium from a first vessel to a second vessel, wherein the fluidic medium comprises a plurality of detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe comprises:
(i) an affinity reagent comprising a binding region and a non-binding region;
(ii) a molecule non-covalently bound to the non-binding region of the affinity reagent; and
(iii) a detectable label attached to the molecule;
(b) binding detectable probes of the plurality of detectable probes to analytes of the plurality of analytes; and
(c) detecting at single-analyte resolution for each site of the plurality of sites a presence or absence of a signal from the detectable label of a detectable probe of the plurality of detectable probes.

2. The method of claim 1, wherein the solid support comprises a plurality of wells, wherein each well comprises only one site of the plurality of sites.

3. The method of claim 2, wherein a solid layer is disposed on the solid support, wherein the solid layer comprises the plurality of wells.

4. The method of claim 1, wherein the affinity reagent comprises an antibody or a fragment thereof.

5. The method of claim 4, wherein the non-binding region comprises an epitope of the Fc region of the antibody.

6. The method of claim 4, wherein the molecule non-covalently bound to the non-binding region of the affinity reagent comprises an antibody or a fragment thereof.

7. The method of claim 4, wherein the molecule non-covalently bound to the non-binding region of the affinity reagent comprises an antibody-binding protein.

8. The method of claim 7, wherein the antibody binding protein is selected from the group consisting of Protein A, Protein G, Protein L, Protein M, and Protein A/G.

9. The method of claim 1, wherein the affinity reagent has a binding specificity for an epitope containing between 2 and 5 amino acids.

10. The method of claim 1, wherein the affinity reagent has a binding specificity for an epitope that is common to two or more different proteins.

11. The method of claim 1, wherein the affinity reagent has a binding specificity for an epitope that contains a post-translational modification of a protein.

12. The method of claim 9, wherein binding detectable probes of the plurality of detectable probes to analytes of the plurality of analytes comprises binding the affinity reagent of a detectable probe of the plurality of detectable probes to the epitope.

13. The method of claim 1, wherein the detectable label of a detectable probe of the plurality of detectable probes comprises a luminescent or fluorescent molecule.

14. The method of claim 13, wherein detecting the signal from the detectable label of a detectable probe of the plurality of detectable probes comprises detecting with an optical detector a photon emitted by the luminescent or fluorescent molecule.

15. The method of claim 1, wherein the detectable probe comprises a plurality of detectable labels.

16. The method of claim 1, further comprising dissociating the detectable probes from the analytes of the plurality of analytes.

17. The method of claim 16, further comprising, after dissociating the detectable probes from the analytes of the plurality of analytes, repeating steps (a)-(c) with the same plurality of detectable probes.

18. The method of claim 17, wherein a second set of analytes is bound by the detectable probes after repeating steps (a)-(c), in which the second set of analytes differs from the analytes bound by the detectable probes during the first cycle.

19. A method, comprising:
(a) delivering a fluidic medium from a first vessel to a second vessel, wherein the fluidic medium comprises a plurality of first detectable probes and a plurality of second detectable probes, wherein the second vessel comprises a solid support, wherein the solid support comprises a plurality of sites, wherein a plurality of analytes is immobilized on the plurality of sites, wherein each site of the plurality of sites contains only one analyte of the plurality of analytes, and wherein each detectable probe of the plurality of first detectable probes and the plurality of second detectable probes comprises:
(i) an affinity reagent comprising a binding region and a non-binding region;
(ii) a molecule non-covalently bound to the non-binding region of the affinity reagent; and
(iii) a detectable label attached to the molecule;
(b) binding detectable probes of the plurality of first detectable probes and plurality of second detectable probes to analytes of the plurality of analytes; and
(c) detecting at single-analyte resolution for each site of the plurality of sites a presence or absence of a signal from the detectable label of a detectable probe of the plurality of first detectable probes and the detectable label of a detectable probe of the plurality of second detectable probes;
wherein the non-binding region of the affinity reagents of the detectable probes of the plurality of first detectable probes differ from the non-binding region of the affinity reagents of the detectable probes of the plurality of second detectable probes; and
wherein the detectable labels of the detectable probes of the plurality of first detectable probes differ from the detectable labels of the detectable probes of the plurality of second detectable probes.

20. A system, comprising:
(a) a first vessel, wherein the first vessel comprises a fluidic medium, wherein the fluidic medium comprises a plurality of detectable probes, wherein each detectable probe comprises:
(i) an affinity reagent comprising a binding region and a non-binding region;
(ii) a molecule non-covalently bound to the non-binding region of the affinity reagent; and
(iii) a detectable label attached to the molecule;
(b) a second vessel comprising a solid support, wherein the solid support comprises a plurality of sites, wherein a plurality of analytes is immobilized on the plurality of sites, wherein each site of the plurality of sites contains only one analyte of the plurality of analytes;
(c) a fluid transfer device, wherein the fluid transfer device provides fluidic communication between the first vessel and the second vessel;
(d) an optical detector; and
(e) one or more processors programmed to read data from the optical detector to detect presence or absence of a signal from the detectable label at each site of the plurality of sites at single-analyte resolution.
